(19) United States
(12) Patent Application Publication
(10) Pub. No.: US 2025/0197874 A1
(43) Pub. Date: Jun. 19, 2025

(54) PLANT-PRODUCED CHIMAERIC ORBIVIRUS VLPS

(71) Applicants: CSIR, Pretoria (ZA); UNIVERSITY OF CAPE TOWN, Cape Town (ZA); ONDERSTEPOORT BIOLOGICAL PRODUCTS SOC LTD, Pretoria (ZA)

(72) Inventors: Albertha René VAN ZYL, Wynberg (ZA); Ann Elizabeth MEYERS, Plumstead (ZA); Daria Anna RUTKOWSKA, Centurion (ZA); Edward Peter RYBICKI, Cape Town (ZA); Hester Catharina STARK, Irene (ZA); Martha Magaretha O'KENNEDY, Pretoria (ZA); Nobalanda Betty MOKOENA, Montana (ZA)

(73) Assignees: CSIR, Pretoria (ZA); UNIVERSITY OF CAPE TOWN, Cape Town (ZA); ONDERSTEPOORT BIOLOGICAL PRODUCTS SOC LTD, Pretoria (ZA)

(21) Appl. No.: 18/989,890

(22) Filed: Dec. 20, 2024

Related U.S. Application Data
(63) Continuation of application No. 17/335,404, filed on Jun. 1, 2021, now Pat. No. 12,215,328.
Application No. 17/335,404 is a division of application No. 16/095,264, filed on Oct. 19, 2018, now Pat. No. 11,053,509.

(30) Foreign Application Priority Data
Apr. 19, 2016 (ZA) 2016/02705

(51) Int. Cl. C12N 15/82; A61K 35/76; A61K 39/00; A61K 39/12; A61P 31/14

(52) U.S. Cl. CPC C12N 15/8205; A61K 35/76; A61K 39/12; A61P 31/14; C12N 15/8258; A61K 2039/5258; C12N 2720/12134

(57) ABSTRACT

This invention relates to a second generation, plant-produced synthetic Orbivirus candidate vaccine. The vaccine comprises a plant produced chimaeric Orbivirus virus like particle (VLP) comprising at least one structural protein from one Orbivirus serotype and at least one structural protein selected from another serotype of the Orbivirus, wherein both structural capsid proteins are from the same Orbivirus species. In particular the invention relates to a vaccine against an Orbivirus, a method of producing chimaeric Orbivirus virus-like particles (VLPs) for use in a method of prevention and/or treatment of an Orbivirus infection, the use of the chimaeric Orbivirus VLPs in the manufacture of a vaccine for an Orbivirus, and a method of preventing and/or treating an Orbivirus infection.

### CROSS REFERENCE TO RELATED APPLICATIONS

The present application is a continuation application of allowed, co-pending U.S. application Ser. No. 17/335,404 which was filed Jun. 1, 2021 and which is a divisional application of U.S. application Ser. No. 16/095,264, currently U.S. Pat. No. 11,053,509 issued July 6 2021, which was filed under 35 U.S.C. § 371 as the U.S. national phase of International Patent Application No. PCT/IB2017/052236, filed Apr. 19, 2017, which designated the United States and claims priority to South African Patent Application No. 2016/02705, filed Apr. 19, 2016, each of which is hereby incorporated in its entirety including all tables, figures and claims.

### SEQUENCE LISTING

The application herein incorporates by reference in its entirety the sequence listing material submitted concurrently with the specification as an XML file, with a filename of “11170.001US3” a creation date of Dec. 20, 2024, and a size of 167,936 bytes. The ST26 Sequence Listing is part of the specification and is incorporated in its entirety by reference herein.

### BACKGROUND OF THE INVENTION

This invention relates to a second generation, plant-produced synthetic Orbivirus candidate vaccine. The vaccine comprises a plant produced chimaeric Orbivirus VLP having a common core comprising at least one structural protein from one Orbivirus serotype and an outer layer comprising at least one structural protein selected from another serotype of the Orbivirus, wherein both structural capsid proteins are from the same Orbivirus species. In particular the invention relates to a vaccine against an Orbivirus, a method of producing chimaeric Orbivirus virus-like particles (VLPs) for use in a method of prevention and/or treatment of an Orbivirus infection, the use of the chimaeric Orbivirus VLPs in the manufacture of a vaccine for an Orbivirus, and a method of preventing and/or treating an Orbivirus infection.

The invention relates to a chimaeric Orbivirus VLP vaccine which is adaptable so that it can be used to immunize animals against multiple serotypes of an Orbivirus by using a common core comprising at least one capsid protein from one Orbivirus serotype and an outer layer comprising at least one Orbivirus capsid protein from another serotype of the same species of Orbivirus. VLPs which are representative of different serotypes of the same species of Orbiviruses could be mixed and administered in combination as a multivalent vaccine. Using the chimaeric VLPs of the invention it is possible to produce a multivalent vaccine which represents all of the serotypes for a particular Orbivirus species. The plant-produced Orbivirus candidate vaccines of the present invention are particulate in nature and hence stimulate a strong cellular immune response.

The genus Orbivirus is a member of the Reoviridae family, in the subfamily Sedoreovirinae. Unlike the other reoviruses, Orbiviruses are arboviruses and, as such, are transmitted by arthropod vectors. The Orbivirus genus currently contains 22 species and at least 130 different serotypes. Orbiviruses can infect and replicate within a wide range of arthropod and vertebrate hosts. Many Orbiviruses are transmitted by ticks or haematophagus insect vectors (Culicoides spp, mosquitoes and sand flies) and have a wide host range that includes cattle, goats and sheep, wild ruminants, equids, camelids, marsupials, sloths, bats, birds, large canine and feline carnivores and humans.

The Orbivirus virions are non-enveloped particles that are between 70-80 nm in diameter. These virus particles are spherical in appearance and are arranged as an icosahedral structure made up of three concentric layers of 4 major structural proteins (VPs) arranged in concentric shells around the double-stranded RNA genome and other minor structural and non-structural proteins. Outer, intermediate and an inner capsid layers surround the genome, with the intermediate and inner capsids having T=13 and T=2 symmetry, respectively. The core is constructed of two concentric protein shells, the inner capsid layer which contains 120 VP3 protein copies and the intermediate capsid layer composed of 780 VP7 protein copies. VP1, VP4 and VP6 are minor enzymatic proteins that are packaged along with the ten genome segments within the central space of the virus core. The Orbivirus outer capsid layer is composed of two additional structural proteins, VP2 and VP5, which mediate viral cell-attachment and penetration, respectively, during initiation of infection. The outer capsid proteins are more variable than the core proteins, and most of the non-structural proteins, and the specificity of their reactions with neutralising antibodies determines the virus serotype.

Orbiviruses have double stranded RNA genomes and are classified as Class Ill viruses. Their genome is linear and is segmented into ten segments of various lengths. One copy of each gene segment is packaged per virion. In most cases each gene segment encodes a single open reading frame (ORF). The genome encodes the seven structural proteins (VP1-VP7) and the four non-structural proteins (NS1, NS2, NS3/3A, NS4).

Many Orbiviruses preferentially infect vascular endothelial cells. Orbiviruses enter the host cell by endocytosis and the outer capsid is subsequently removed. The whole cycle of viral replication takes place within the cytoplasm of the host cell. Transcription of the viral genome into mRNA occurs within the core particle and mRNA is translated into proteins using the host cell ribosomes. Viral proteins are synthesized 2-14 days after initial infection. New virons self-assemble within the cytoplasm and are then released from the host cell by budding. During the budding process they transiently acquire a lipid envelope which can be detected for a short period of time following their release but this is subsequently lost.

It has been shown that co-expression of the 4 Orbivirus capsid proteins in a recombinant baculovirus system results in the formation of virus-like particles (VLPs). When VP3 and VP7 are co-expressed, 60 dimers of VP3, the innermost protein, assemble into a particle—these particles are called subcore-like particles (SCLPs); trimers of VP7 then form an icosahedral shell on the VP3 scaffold resulting in stable core-like particles (CLPs). However, these are not immunogenic in animals, which is not surprising as they do not contain the neutralizing protein VP2 which presents the major immunogenic determinants. A third shell is formed when the VP5 and VP2 of the same Orbivirus are co-expressed with the VP3 and VP7 proteins. VP2 trimers position themselves on the CLP surface and are interspersed with VP5 trimers. BTV VLPs formed in this manner have been shown to be immunogenic in sheep. Thus, Orbivirus VLPs may be an inherently safe and effective vaccine. Orbivirus VLPs have previously been expressed in insect cells, however this method of production remains costly.

Expression of recombinant proteins in plants has developed over the last twenty years from a curiosity in the late 1980s to a medically and industrially relevant production system today. Early efforts relied on transformation of plants to produce stable transgenic lines. This was achieved through biolistic delivery or, more recently, agroinfiltration. While transgenic protein production remains a useful and viable system, advances in transient expression methods and technology have positioned transient expression as the preferred method for industrial-scale production in plants. Two key factors that have played a central role in this transition are viral, or virus-derived, expression vectors and the development of agroinfiltration technology.

Agroinfiltration was originally developed as a means of introducing foreign DNA into plant cells for transient expression of recombinant proteins. This process relies on the DNA transfer capability of Agrobacterium tumefaciens to introduce foreign DNA into plant cells. A. tumefaciens can be used to transfer a transgene located in the transfer DNA (T-DNA) segment of the Ti plasmid into plants infiltrated with a bacterial suspension of the transformed bacterium. The T-DNA is transported to the plant nucleus but is rarely integrated into the plant genome. Instead, it exists as an episome from which transcription and translation of genes of interest cloned into the T-DNA take place, allowing for transient expression of recombinant proteins of interest. Viral vectors were the first transient expression method developed for plants. Early efforts simply inserted a recombinant gene or epitope into the genome of viruses such as TMV, cowpea mosaic virus (CPMV), or PVX, either fused to the viral coat protein or separately, under control of a duplicated constitutive viral promoter. While this application produced immunogenic proteins, expression levels were lower than those found in transgenic plants. Other problems with these ‘first-generation’ viral vectors included a tendency to revert to the natural virus, constraints on insert size, difficulty of administration, and an inability to form VLPs.

These limitations prompted further work to develop ‘second generation’, or deconstructed, viral vectors. This approach used only the desirable viral elements, in particular the replicative machinery, to manufacture synthetic vectors capable of inducing transgene expression in plants. While these vectors are usually not infectious on their own, when coupled with agroinfiltration technology they can result in systemic transient expression of protein at levels comparable to that of transgenic plants. This approach has the advantages of short time frames (3-7 days) when compared to stable transformation (6-9 months), significant expression levels, and rapid and easy scale-up and purification. This makes agroinfiltration-mediated transient expression via viral vectors an ideal approach for the production of medically relevant proteins and particles in plants. Of particular interest is the use of transient expression for the production of VLPs in plants, as there is potential for a reduction in cost when compared to traditional systems.

Using the above mentioned technologies the applicant has developed a plant-produced Orbivirus VLP vaccine consisting of a mixture of chimaeric VLPs, as discussed herein each VLP consists of a core comprising of at least one capsid protein from one Orbivirus serotype with an outer layer consisting of at least one capsid protein from another serotype of the same Orbivirus species.

Since the VP2 capsid protein contains the neutralising epitopes, mixtures of different chimaeric VLPs provide a plant-produced particulate vaccine which can be used in animals to protect them against more than one serotype of the same Orbivirus species. The value of this product lies in the low safety requirements for production, as well as the cost effectiveness of the production method.

Virus-like particles (VLPs) are considered excellent immunogens for a number of reasons: they resemble the mature viral particles in size and shape but lack the viral genome and are thus non-replicating and non-infectious; they have also been shown to stimulate both the humoral and cellular arms of the immune system. The repetitive nature of protein epitopes exposed on the surfaces of VLPs means they stimulate a strong antibody response as a result of B cells recognising specific repetitive units.

There are a number of other advantages to using VLPs as vaccine candidates. In the case of some VLPs (eg: Human papillomavirus-HPV) it has been shown that co-administration of an adjuvant is not required for the induction of a strong antibody response, thus reducing the vaccine dose costs. VLPs can be used to broaden the protective ability of the vaccine with the inclusion of additional epitopes which would lower vaccine dose amounts and concomitant vaccine costs. In the case of animal vaccines, VLP vaccines can be designed so as to exclude markers used for diagnosis of viral infection, allowing for the distinction between infected and vaccinated animals (DIVA): this is a very important requirement in areas affected by disease outbreaks such as in the EU. These factors have led to rapid advances in the field of VLP vaccine development and production. Accordingly, this invention aims to provide non-infectious, non-replicating vaccines against Orbviruses.

The three most economically important Orbiviruses are Bluetongue virus, African horse sickness virus and epizootic hemorrhagic disease virus, all of which are transmitted by Culicoides species. Bluetongue (BT) disease is a non-contagious, insect-borne, viral disease of ruminants, mainly sheep and less frequently cattle, goats, buffalo, deer, dromedaries, and antelope. In sheep, BT disease causes an acute disease with high morbidity and mortality, with up to 90% mortality in some breeds. Bluetongue disease is caused by Bluetongue virus (BTV), of the genus Orbivirus, of the Reoviridae family. There are twenty-six recognised serotypes for this virus. Bluetongue has been observed in Australia, the USA, Africa, the Middle East, Asia and Europe. There is no efficient treatment. Prevention is effected via quarantine, inoculation with live modified virus vaccine and control of the midge vector. The existing BTV vaccine used in South Africa consists of field strains of BTV attenuated through serial passage in embryonated chicken eggs and BHK-21 cells. It consists of 3 bottles (A, B and C) each containing 5 different serotypes (A-1, 4, 6, 12 and 14; B-3, 8, 9, 10 and 11; C-2, 5, 7, 13 and 19). This does not represent all the BTV serotypes circulating in South Africa, which total 26. The reason for this is that the missing types do not cause very severe pathogenicity in sheep (serotypes 15, 16, 18, 22-26). The production process in eggs and cell culture is costly and the inclusion of so many different serotypes would also add significantly to the cost.

It has been shown previously using BTV-10 VP2, VP3, VP5 and VP7 that co-expression of these four structural proteins in insect cells results in the formation of VLPs. They were shown to induce protective immunity in sheep which were challenged with live BTV-10 virus. Similar VLPs made in insect cells by co-expression of the four structural genes representing a different BTV serotype (BTV-1) have also been shown to induce neutralizing antibodies and protect sheep challenged with BTV-1. It has however, not previously been shown that co-expression of structural proteins from two different serotypes of the same Orbivirus will form chimaeric VLPs when co-expressed in a plant cell.

African horse sickness (AHS) is an infectious, non-contagious disease of equids, with a mortality rate of up to 95% in horses, the most susceptible species. African horse sickness is caused by African horse sickness virus (AHSV), which is transmitted by Culicoides midges. AHSV is classified as an Orbivirus in the family Reoviridae and there are currently nine recognised AHSV serotypes, namely serotypes 1 to 9. Although endemic to Sub-Saharan Africa, devastating sporadic outbreaks of AHS disease have also occurred in North Africa, the Iberian Peninsula, the Middle East and Asia. Currently African horse sickness is classified by the World Organization for Animal Health (OIE) as a notifiable disease, and strict quarantine measures govern the transport of horses from endemic countries, such as South Africa, to non-endemic regions. AHS outbreaks not only have significant economic implications on the equine industries of affected countries, but also impact directly on the agricultural and transport activities of rural communities, particularly in South Africa. More than 50% of the horses in South Africa are estimated to belong to rural community horse owners.

In endemic regions, annual prophylactic vaccination of horses with a commercial live attenuated vaccine (Onderstepoort Biological products (OBP)) is an efficient way of preventing serious losses during the peak AHS season. The multivalent vaccine consists of two components, the trivalent component (serotypes 1, 3 and 4) and the quadrivalent component (serotypes 2, 6, 7 and 8) administered three weeks apart. Although serotypes 5 and 9 are excluded from the multivalent vaccine formulation, in vivo cross-protection afforded against serotype 5 by the included serotype 8 and cross-protection afforded by the included serotype 6 against serotype 9, ensure that the vaccine is effective against all AHS serotypes. There are, however, several drawbacks associated with the use of the current AHS vaccine and these include the risk of reversion to virulence, teratogenic effects in pregnant mares as well as the inability to differentiate between infected and vaccinated animals (DIVA). These concerns have prohibited the use of this vaccine in non-endemic areas. Production of a live attenuated vaccine requires high biosafety levels during manufacture, thus elevating cost, and also precludes a rapid production rate. A plant-produced AHS VLP-based vaccine would address these concerns. Although AHS VLPs have recently been assembled in insect cells, the complexity of production and upscaling in insect cells will make only a monovalent AHS VLP based vaccine (single serotype) possible.

Whilst live attenuated vaccines for BT and AHS are available, safety concerns have prohibited the use of these vaccines in non-endemic areas, such as Europe. The use of live virus also makes it difficult to differentiate infected from vaccinated animals (DIVA). Production of live vaccines requires high biosafety levels for handling, thus elevating the production cost. The use of live virus also precludes a rapid production rate. Alternatives to the current live attenuated vaccine include an inactivated vaccine, viral vectors expressing the outer capsid proteins and subunit vaccines. Plant produced chimaeric Orbivirus VLP vaccines will abrogate the need for high biosafety levels during manufacture. In addition, VLPs can be made more rapidly by transient expression in agroinfiltrated plants.

### SUMMARY OF THE INVENTION

The present invention provides for chimaeric Orbivirus VLPs which are immunogenic and which are useful in the formulation of a vaccine composition against Orbivirus infection in a subject. The invention relates to the chimaeric Orbivirus VLPs, methods for their production and vaccine compositions containing the chimaeric Orbivirus VLPs.

In one embodiment, the present invention provides for genetically engineered Orbivirus VLPs comprising a single chimaeric Orbivirus VLP, a double chimaeric Orbivirus VLP, a triple chimaeric Orbivirus VLP or a quadruple chimaeric Orbivirus VLP. It will be appreciated that in these chimaeric Orbivirus VLPs at least one structural protein is selected from one serotype of a specific Orbivirus species and the other structural proteins are selected from at least one other serotype of the same species of Orbivirus.

For instance, exemplary, non-limiting, genetically engineered BTV VLPs of the present invention may be a single chimaeric if they comprise, for instance, of one structural protein from a first BTV serotype i.e. a BTV-2 VP2 and the other structural proteins from a second BTV serotype i.e. BTV-8 VP3, BTV-8 VP5 and BTV-8 VP-7. Similarly, an exemplary double chimaeric BTV VLP of the present invention will comprise two structural proteins from a first BTV serotype and two structural proteins from a second BTV serotype, for instance, BTV-8 VP3, BTV-8 VP7, BTV-3 VP2 and BTV-3 VP5. An exemplary triple chimaeric BTV VLP of the invention may comprise, two structural proteins from a first BTV serotype, one structural protein from a second BTV serotype and one structural protein from a third BTV serotype, for instance, BTV-8 VP3, BTV-8 VP7, BTV-3 VP2 and BTV-4 VP5. Likewise, an exemplary quadruple chimaeric BTV VLP of the present invention may comprise, one structural protein from a first BTV serotype, one structural protein from a second BTV serotype, one structural protein from a third BTV serotype and one structural protein from a fourth BTV serotype, for instance, BTV-2 VP2, BTV-3 VP5, BTV-4 VP7 and BTV-8 VP3.

Exemplary, non-limiting, genetically engineered AHSV VLPs of the present invention may be a single chimaeric if they comprise, for instance, of one structural protein from a first AHSV serotype, for instance, an AHSV-7 VP2 and the other structural proteins from a second AHSV serotype, for instance, AHSV-1 VP3, AHSV-1 VP5 and AHSV-1 VP7. Similarly, an exemplary double chimaeric AHSV VLP of the present invention will comprise two structural proteins from a first AHSV serotype and two structural proteins from a second AHSV serotype, for instance, AHSV-1 VP3, AHSV-1 VP7, AHSV-7 VP2 and AHSV-7 VP5. An exemplary triple chimaeric AHSV VLP of the invention may comprise, two structural proteins from a first AHSV serotype, one structural protein from a second AHSV serotype and one structural protein from a third AHSV serotype, for instance, AHSV-1 VP3, AHSV-1 VP7, AHSV-6 VP2 and AHSV-3 VP5. Likewise, an exemplary quadruple chimaeric AHSV VLP of the present invention may comprise, one structural protein from a first AHSV serotype, one structural protein from a second AHSV serotype, one structural protein from a third AHSV serotype and one structural protein from a fourth AHSV serotype, for instance, AHSV-6 VP2, AHSV-3 VP5, AHSV-2 VP7 and AHSV-1 VP3.

Those skilled in the art will appreciate that chimaeric single, double, triple and quadruple Orbivirus VLPs can be constructed for any Orbivirus species using the methods described herein and as exemplified for BTV and AHSV.

The invention also relates to a multivalent vaccine composition comprising a combination of the chimaeric Orbivirus VLPs, wherein the chimaeric VLPs comprise structural proteins representative of several different serotypes of a particular Orbivirus species.

According to a first aspect of the invention there is provided for a chimaeric Orbivirus VLP comprising at least one structural protein from a first serotype of an Orbivirus species and at least one structural protein from a second serotype of an Orbivirus species. The chimaeric Orbivirus VLP of the invention is produced in and recovered from a plant cell.

In a preferred embodiment of the invention there is provided for a chimaeric Orbivirus virus-like particle (VLP) comprising VP2, VP3, VP5 and VP7 structural proteins, wherein at least one of the VP2, VP3, VP5 and VP7 structural proteins is selected from a first Orbivirus serotype and at least one of the VP2, VP3, VP5 and VP7 structural proteins is selected from a second Orbivirus serotype, wherein the Orbivirus serotypes are of the same Orbivirus species, and wherein the chimaeric Orbivirus VLP is produced according to a method comprising the steps of (i) providing codon-optimised nucleotide sequences encoding the Orbivirus VP2, VP3, VP5 and VP7 structural proteins, (ii) cloning the codon-optimised nucleotide sequences into at least one expression vector adapted to express the structural proteins in a plant cell, (iii) transforming or infiltrating the plant cell with the at least one expression vector of step (ii), co-expressing the VP2, VP3, VP5 and VP7 structural proteins in the plant cell, such that the expressed structural proteins assemble to form the chimaeric Orbivirus VLP; and (v) recovering the chimaeric Orbivirus VLP from the plant cell.

In one preferred embodiment of the invention the chimaeric Orbivirus VLP comprises at least one of the VP2, VP3, VP5 and VP7 structural proteins from a third Orbivirus serotype of the same Orbivirus species. Alternatively, at least one of the VP2, VP3, VP5 and VP7 structural proteins may be from a fourth Orbivirus serotype of the same Orbivirus species.

The chimaeric Orbivirus VLP of the invention may be a single chimaeric Orbivirus VLP comprising a first VP2, VP3, VP5 or VP7 structural protein from a first Orbivirus serotype and the other three structural proteins from a second Orbivirus serotype. Alternatively, the chimaeric Orbivirus VLP of the invention may be a double chimaeric Orbivirus VLP comprising two of the VP2, VP3, VP5 or VP7 structural proteins from the first Orbivirus serotype and two of the structural proteins from the second Orbivirus serotype. Further alternatively, the chimaeric Orbivirus VLP may be a triple chimaeric Orbivirus VLP comprising two of the VP2, VP3, VP5 or VP7 structural proteins from a first Orbivirus serotype, one structural protein from a second Orbivirus serotype, and one structural protein from a third Orbivirus serotype. Alternatively, the chimaeric Orbivirus VLP may be a quadruple chimaeric Orbivirus VLP comprising the first VP2, VP3, VP5 or VP7 structural protein from a first Orbivirus serotype, the second structural protein from a second Orbivirus serotype, the third structural protein from a third Orbivirus serotype, and the fourth structural protein from a fourth Orbivirus serotype.

In another preferred embodiment the plant or plant cell is Nicotiana benthamiana plant or plant cell. In yet a further preferred embodiment the plant or plant cell may be a N. benthamiana dXT/FT mutant tobacco plant or cell, which facilitates mammalian-like or human-like glycosylation of the polypeptides.

In one embodiment of the invention, for instance, where the Orbivirus is BTV the first serotype is, for instance, BTV-8 then the second Orbivirus serotype may be a BTV serotype selected from the group consisting of BTV-1, BTV-2, BTV-3, BTV-4, BTV-5, BTV-6, BTV-7, BTV-9, BTV-10, BTV-11, BTV-12, BTV-13, BTV-14, BTV-15, BTV-16, BTV-17, BTV-18, BTV-19, BTV-20, BTV-21, BTV-22, BTV-23, BTV-24, BTV-25, BTV-26 and BTV-27.

In another embodiment of the invention, for instance, where the Orbivirus is AHSV the first serotype is, for instance, AHSV-1 then the second Orbivirus serotype is an AHSV serotype selected from the group consisting of AHSV-2, AHSV-3, AHSV-4, AHSV-5, AHSV-6, AHSV-7, AHSV-8 and AHSV-9.

The chimaeric BTV VLPs or chimaeric AHSV VLPs of the embodiments described above are produced in a plant cell which has been transformed with one or more vectors that regulate the expression of the VP2, VP3, VP5 and VP7 structural proteins. It will be understood by those of skill in the art that each of the genes encoding the structural proteins may each be contained on a separate vector. Alternatively, two or more of the genes encoding the structural proteins may be contained on a single vector, in any combination. Preferably, in order to facilitate the formation of VLPs in the plant cells, the vectors containing the genes encoding the structural proteins will be transformed into the plant cell in a ratio of 1:1:1:1 or a ratio of 1:1:2:1 or a ratio of 2:1:2:1 of the genes encoding VP2:VP3:VP5:VP7.

In one preferable embodiment, the plant cell is transformed using Agrobacterium-mediated transformation. Most preferably, the expression of the Orbivirus VP2, VP3, VP5 and VP7 structural proteins in the plant cell is mediated by the Agrobacterium, wherein the Agrobacterium is selected from Agrobacterium AGL-1, Agrobacterium LBA4404, Agrobacterium GV3101 pMP90 or any other suitable agrobacterial strain.

In a further preferable embodiment the VP2, VP3, VP5 and VP7 structural proteins are transiently co-expressed in the plant cell. However, those of skill in the art will appreciate that stable transformation of the plant cell will also lead to the formation of chimaeric Orbivirus VLPs.

It will be appreciated that the embodiments described above relate to BTV and AHSV, however those of skill in the art will appreciate that using the same approach it will be possible to produce Orbivirus VLPs from any species of Orbivirus. Specifically, the Orbivirus species may be selected from the group consisting of Lebombo virus (LEBV), Pata virus (PATAV), African horse sickness virus (AHSV), Bluetongue virus (BTV), Altamira virus (ALTV), Almeirim virus (AMRV), Caninde virus (CANV), Changuinola virus (CGLV), Irituia virus (IRIV), Jamanxi virus (JAMV), Jari virus (JARIV), Gurupi virus (GURV), Monte Dourado virus (MDOV), Ourem virus (OURV), Purus virus (PURV), Saraca virus (SRAV), Acado virus (ACDV), Corriparta virus (CORV), Eubenangee virus (EUBV), Ngoupe virus (NGOV), Tilligerry virus (TILV), Epizootic hemorrhagic disease virus (EHDV), Kawanabe virus, Equine encephalosis virus (EEV), Great Island virus, Kemerovo virus (KEMV), Essaouira virus (ESSV), Kala iris virus (KIRV), Mill Door/79 virus (MILDV), Rabbit syncytium virus (RSV), Tribeč virus (TRBV), Broadhaven virus (BRDV), Orungo virus (ORUV), Abadina virus (ABAV), Apies River virus, Bunyip Creek virus (BCV), Chuzan (Kasba) virus (SBV), CSIRO Village virus (CVGV), D'Aguilar virus (DAGV), Marrakai virus (MARV), Petevo virus (PETV), Vellore virus (VELV), Llano Seco virus (LLSV), Minnal virus (MINV), Netivot virus (NETV), Umatilla virus (UMAV), Wallal virus (WALV) and Mitchell River virus (MRV).

According to a second aspect of the invention there is provided for a method of producing a chimaeric Orbivirus VLP in a plant cell, comprising transforming the plant cell with one or more vectors that regulate the expression of, for example, VP3, VP5 and VP7 structural proteins from a first Orbivirus serotype and VP2 from a second Orbivirus serotype and expressing the VP2, VP3, VP5 and VP7 structural proteins in the plant cell, wherein the expressed VP2, VP3, VP5 and VP7 structural proteins assemble to form a chimaeric Orbivirus VLP. The method further provides for transient co-expression of the VP2, VP3, VP5 and VP7 structural proteins in the plant cell. The method further provides a step of recovering the chimaeric Orbivirus VLP from the plant cell.

In a preferred embodiment the method comprises a method of producing a chimaeric Orbivirus VLP in a plant cell, the method comprising the steps of (i) providing codon-optimised nucleotide sequences encoding Orbivirus VP2, VP3, VP5 and VP7 structural proteins, wherein at least one of the VP2, VP3, VP5 and VP7 structural proteins is selected from a first Orbivirus serotype and at least one of the VP2, VP3, VP5 and VP7 structural proteins is selected from a second Orbivirus serotype of the same Orbivirus species, (ii) cloning the codon-optimised nucleotide sequences into at least one expression vector adapted to express the structural proteins in a plant cell, (iii) transforming or infiltrating the plant cell with the at least one expression vector of step (ii), (iv) co-expressing the VP2, VP3, VP5 and VP7 structural proteins in the plant cell, such that the expressed structural proteins assemble to form the chimaeric Orbivirus VLP; and (v) recovering the chimaeric Orbivirus VLP from the plant cell.

In one embodiment of the invention at least one of the VP2, VP3, VP5 and VP7 structural proteins of step (i) of the method is selected from a third Orbivirus serotype of the same Orbivirus species. Alternatively, at least one of the VP2, VP3, VP5 and VP7 structural proteins is selected from a fourth Orbivirus serotype of the same Orbivirus species.

According to one embodiment of the invention, if the Orbivirus is BTV the first and second BTV serotypes may be selected from the group consisting of BTV-1, BTV-2, BTV-3, BTV-4, BTV-5, BTV-6, BTV-7, BTV-8 BTV-9, BTV-10, BTV-11, BTV-12, BTV-13, BTV-14, BTV-15, BTV-16, BTV-17, BTV-18, BTV-19, BTV-20, BTV-21, BTV-22, BTV-23, BTV-24, BTV-25, BTV-26 and BTV-27. According to another embodiment of the invention, if the Orbivirus is AHSV the first and second AHSV serotypes may be selected from the group consisting of AHSV-1, AHSV-2, AHSV-3, AHSV-4, AHSV-5, AHSV-6, AHSV-7, AHSV-8 and AHSV-9.

In yet another aspect of the invention it will be appreciated that if the Orbivirus is selected from one of the Orbiviruses in the group comprising Lebombo virus (LEBV), Pata virus (PATAV), African horse sickness virus (AHSV), Bluetongue virus (BTV), Altamira virus (ALTV), Almeirim virus (AMRV), Caninde virus (CANV), Changuinola virus (CGLV), Irituia virus (IRIV), Jamanxi virus (JAMV), Jari virus (JARIV), Gurupi virus (GURV), Monte Dourado virus (MDOV), Ourem virus (OURV), Purus virus (PURV), Saraca virus (SRAV), Acado virus (ACDV), Corriparta virus (CORV), Eubenangee virus (EUBV), Ngoupe virus (NGOV), Tilligerry virus (TILV), Epizootic hemorrhagic disease virus (EHDV), Kawanabe virus, Equine encephalosis virus (EEV), Great Island virus, Kemerovo virus (KEMV), Essaouira virus (ESSV), Kala iris virus (KIRV), Mill Door/79 virus (MILDV), Rabbit syncytium virus (RSV), Tribeč virus (TRBV), Broadhaven virus (BRDV), Orungo virus (ORUV), Abadina virus (ABAV), Apies River virus, Bunyip Creek virus (BCV), Chuzan (Kasba) virus (SBV), CSIRO Village virus (CVGV), D′Aguilar virus (DAGV), Marrakai virus (MARV), Petevo virus (PETV), Vellore virus (VELV), Llano Seco virus (LLSV), Minnal virus (MINV), Netivot virus (NETV), Umatilla virus (UMAV), Wallal virus (WALV) and Mitchell River virus (MRV) then the first and second serotypes may be selected from different serotypes of the same species of Orbivirus.

In instances where a double, triple or quadruple chimaeric Orbivirus of the invention is produced it will be appreciated that the structural proteins will be selected from two, three or four different serotypes of a particular Orbivirus species, respectively.

It will be appreciated that the at least one expression vector described in the method includes a promoter and/or other regulatory sequences, operably linked to each nucleotide sequence encoding each structural protein.

In another embodiment the plant or plant cell is Nicotiana benthamiana plant or plant cell. In yet a further preferred embodiment the plant or plant cell may be a N. benthamiana dXT/FT mutant tobacco plant or cell, which facilitates mammalian-like or human-like glycosylation of the polypeptides. The plant cell is preferably transformed using Agrobacterium-mediated transformation. Most preferably, the expression of the Orbivirus VP2, VP3, VP5 and VP7 structural proteins in the plant cell is mediated by the Agrobacterium, and the Agrobacterium may be selected from Agrobacterium AGL-1, Agrobacterium LBA4404, Agrobacterium GV3101 pMP90 or any other suitable agrobacterial strain.

A further aspect of the invention provides for a vaccine composition comprising a chimaeric Orbivirus VLP and a pharmaceutically acceptable diluent or excipient, wherein the vaccine composition is capable of eliciting a protective immune response against a specific Orbivirus species in a subject. Preferably the immune response is a cellular and/or humoral immune response.

In a preferred embodiment of the invention the vaccine composition comprises at least one chimaeric Orbivirus VLP of the invention or made by the method of the invention and wherein the vaccine composition elicits a protective immune response against at least one serotype of a specific Orbivirus species in a subject.

A preferred embodiment of the invention provides for a vaccine composition, preferably a multivalent vaccine composition, that comprises a combination of the chimaeric Orbivirus VLPs of the invention. In particular the combination will include at least two different chimaeric VLPs of the invention having structural proteins from the different Orbivirus serotypes from Orbiviruses of the same species. As a result the vaccine composition may comprise VLPs with VP2 structural proteins from more than one serotype of a specific Orbivirus (e.g. BTV or AHSV) the vaccine composition will provide multivalent protection against more than one serotype of BTV, AHSV or a specified species of Orbivirus.

In yet a further embodiment of the invention there is provided for the vaccine composition inducing a protective immune response against an Orbivirus infection in a subject.

The present invention also provides for a method of preventing or treating an Orbivirus infection in a subject, wherein the method comprises a step of administering the chimaeric Orbivirus VLP of the invention or made by the method of the invention to the subject.

A further aspect of the invention provides for the use of the chimaeric Orbivirus VLP of the invention or made according to the method of the invention in the manufacture of a vaccine for use in prevention or treatment of an Orbivirus infection in a subject.

According to a further aspect of the invention there is provided the chimaeric Orbivirus VLP of the invention or made according to the method of the invention for use in a method of preventing or treating Orbivirus infection in a subject, the method comprising administering the chimaeric Orbivirus VLP to the subject. Alternatively the chimaeric Orbivirus VLP of the invention or made according to the method of the invention may be for use in protecting a subject from an Orbivirus infection.

In yet a further aspect of the invention there is provided for a transformed plant cell comprising at least one expression vector adapted to express a codon optimised nucleotide sequence encoding Orbivirus VP2, VP3, VP5 and VP7 structural proteins, wherein at least one of the VP2, VP3, VP5 and VP7 structural proteins is selected from a first Orbivirus serotype and at least one of the VP2, VP3, VP5 and VP7 structural proteins is selected from a second Orbivirus serotype, and wherein the Orbivirus serotypes are of the same Orbivirus species.

In one embodiment the expression of the Orbivirus VP2, VP3, VP5 and VP7 structural proteins in the plant cell is mediated by Agrobacterium AGL-1, Agrobacterium LBA4404, Agrobacterium GV3101 pMP90 or any other suitable agrobacterial strain.

### BRIEF DESCRIPTION OF THE FIGURES

Non-limiting embodiments of the invention will now be described by way of example only and with reference to the following figures:

FIG. 1: Western blot analysis of density gradient fractions. Fractions 1 to 8 from 30 to 60% iodixanol gradient centrifugation of pEAQ-HT VLPs probed with anti-BTV-8 antiserum. PageRuler™ Prestained Protein Ladder (Thermo Scientific™) was used as a size marker.

FIG. 2: TEM analysis of fraction 4 taken from 30 to 60% iodixanol gradient. (a) 14 500× magnification of BTV-8 VLPs and CLPs ranging in size from 70 to 88 nm in diameter. (b) A 50 000× magnification of the same view. Black arrows indicate VLPs measuring 80 nm in size, the white arrow shows a CLP of 65 nm and the empty white arrow indicates a subcore-like particle (measuring 53 nm). Scale bars: (a) 500 nm and (b) 200 nm. The images were obtained using a Technai™ G² transmission electron microscope.

FIG. 3: A TEM of a leaf section infiltrated with only infiltration media at 17000× magnification. B(i) TEM of leaf section showing a mixed population of BTV-8 CLPs and VLPs at a 17000× magnification, B(ii) is a 40 000× magnification of B (i) displaying the particles in more detail.

FIG. 4: Schematic representations of BTV proteins indicating how many copies of each structural protein come together to form subcore-, core- and virus-like particles (SCLP, CLP, VLP). (Thuenemann et al., 2013)

FIG. 5: BTV-like particles for serotype 8. Scale bar, 100 nm (Thuenemann et al., 2013)

FIG. 6: A schematic of the BTV particle showing the four structural proteins, the transcriptase complex and the dsRNA genome (Mertens et al., 2004).

FIG. 7: Western blot of fractions 1 to 8 from 20-60% Optiprep® gradient.

FIG. 8: Electron microscopy of plant-made BTV-like particles and assembly intermediates consisting of (A) serotype 2 and 8 capsid proteins i.e. BTV-2 VP2, BTV-8 VP5, BTV-8 VP3 and BTV-8 VP7 and (B) serotype 8 only capsid proteins i.e BTV-8 VP2, BTV-8 VP5, BTV-8 VP3 and BTV-8 VP7. Arrows indicate fully-formed VLPs.

FIG. 9: Electron microscopy of plant-made BTV-like particles and assembly intermediates consisting of (A) serotype 2 and 8 capsid proteins i.e. BTV-2 VP2, BTV-8 VP5, BTV-8 VP3 and BTV-8 VP7 and (B) serotype 8 only capsid proteins i.e BTV-8 VP2, BTV-8 VP5, BTV-8 VP3 and BTV-8 VP7. Arrows indicate fully-formed VLPs.

FIG. 10: Electron microscopy of chimaeric BTV subcore-like and core-like particles (BRU (2015)) and BTV-8 subcore-like and core-like particles (Thuenemann et al., 2013)

FIG. 11: Western blot and cognate Coomassie-stained gel of fractions 1 to 8 from 20-60% Optiprep™ gradient purification of BTV VP proteins from leaves infiltrated at a 1:1:3:1 (VP3:VP7:VP5:VP2) infiltration ratio.

FIG. 12: SDS-PAGE 4-12% Bolt™ precast gels: lane 1, SeeBlue® Plus molecular marker; lanes 2-3, BTV-8 CLPs and VLPs respectively, positive controls; lanes 4-5, BTV-8 VLPs 45% and 40% sucrose gradient fractions; lanes 6-7, BTV-8 VLPs created with VP3 wt, 45% and 40%; lanes 8-9, BTV-3 single chimaeric, 45% and 40%; lanes 10-11, BTV-3 single chimaeric, BTV-8 VP3 wt core, 45% and 40%; lanes 12-13, BTV-3 double chimaeric, 45% and 40%; lanes 14-15, BTV-3 double chimaeric, BTV-8 VP3wt core, 45% and 40%. VP3 is indicated by an arrow.

FIG. 13: Transmission electron microscope images of sucrose gradient purified BTV-8 CLPs showing the abundance of BTV-8 CLPs created in N. benthamiana dXT/FT to serve as core for BTV serotypes 3 and 4. Negative staining technique using sodium phosphotungstate onto copper grids and images were visualized with a JEM-2100 Transmission electron microscope (JEOL™).

FIG. 14: SDS-PAGE 4-12% Bolt™ precast gels: lane 1, SeeBlue® Plus molecular marker; lanes 2 and 4, BTV-8 CLPs in N. benthamiana dXT/FT; lane 3, BTV-8 CLPs in N. benthamiana; lane 5, BTV-8 VLPs in N. benthamiana dXT/FT; lane 6, BTV-8 N. benthamiana; lane 7, chimaeric BTV-4 VLPs in N. benthamiana dXT/FT; lane 8, chimaeric BTV-4 VLPs in N. benthamiana (all BTV-4 capsids except BTV-8 VP3); lane 9 & 11, double chimaeric BTV-3 VLPs N. benthamiana dXT/FT in 45% and 40% respectively; lane 10, double chimaeric BTV-3 VLPs in N. benthamiana. Sucrose fraction 45% unless otherwise stated.

FIG. 15: Clustal X alignment of BTV-11 VP2 (SEQ ID NO:81) and BTV-4 VP2 (SEQ ID NO:10) indicating that the two peptide sequences detected by mass spectrometry (highlighted in bold and underlined) are identical.

FIG. 16: Sucrose gradient purified double chimaeric BTV-3 VLPs capsid proteins detected in fraction 45% sucrose and separated by SDS-PAGE 4-12% Bolt™ precast gels. Proteins produced in N. benthamiana (even numbers) and N. benthamiana dXT/FT (odd numbers). Lane 1-2, 4 days after infiltration; lanes 3-4, 5 days; lanes 5-6, 6 days; lanes 7-8, 7 days; and lanes 9-10, 8 days; lanes 11-12, BTV-8 CLPs and lane 13, SeeBlue® Plus molecular marker (Life Technologies, Thermo Fisher Scientific™).

FIG. 17: Transmission electron microscope images of double chimaeric BTV-3 VLPs created in N. benthamiana dXT/FT during days 6-8. BTV-8 CLP created as core in mammalian-like N. benthamiana. Negative staining technique using sodium phosphotungstate onto copper grids and images were visualized with a JEM-2100 Transmission electron microscope (JEOL™).

FIG. 18: Clustal X alignment of BTV-4 VP2 (SEQ ID NO:84) and BTV-10 VP2 (SEQ ID NO:82) with overlap peptide sequences detected by Mass Spectrometry.

FIG. 19: Sucrose gradient purified BTV-4 VLP capsid proteins (BTV-8 VP3; BTV-4 VP2, VP5 and VP7) detected in fractions 50% (even numbers) and 45% (odd numbers) sucrose and separated by SDS-PAGE 4-12% Bolt™ precast gels. Proteins produced in N. benthamiana with VP3 indicated by the arrow. Lane 1, SeeBlue® Plus molecular marker; lanes 4-5, bicine buffer with Sigma™ protease inhibitor (PI); lanes 6-7, capsid proteins in bicine buffer with Roche™ EDTA-free protease inhibitor; lanes 8-9, capsids extracted in bicine buffer with 0.5 mM CaCl₂ and Sigma™ PI; lanes 10-11, capsids extracted in bicine buffer with 0.5 mM CaCl₂ and Roche™ PI.

FIG. 20: Alignment of BTV-4 (protein ID ABB71695.1) (SEQ ID NO:10) and BTV-17 (protein ID CAE51104.1) (SEQ ID NO:83) with overlap peptide sequences detected by mass spectrometry.

FIG. 21: Schematic representations of some of the recombinant pEAQ-AHSV plasmids.

FIG. 22: Agarose gel electrophoresis of the AHSV-1 L2, L3, M6 and S7 gene-specific PCR products. The molecular weight marker (M) was the GeneRuler™ DNA ladder (Thermo Scientific™) with the relevant sizes indicated. The arrows indicate the presence of the L2 (3.2 Kb), L3 (2.7 Kb), M6 (1.5 Kb) and S7 (1.1 Kb) PCR products in lanes 1-4, 5-8, 9-12 and 13-16, respectively.

FIG. 23: Photographic record of N. benthamiana leaves agroinfiltrated with LBA4404-PEAQ-HT (left) and LBA4404-PEAQ-HT-gfp (right) and visualised under UV illumination 8 days post-infiltration.

FIG. 24: Immunoblot detection of AHSV-1 and/or AHSV-7 capsid proteins following sucrose density gradient centrifugation. Nicotiana benthamiana leaves, agroinfiltrated with a combination of the pEAQ-HT-AHSV-1 VP2, PEAQ-express AHSV-1 VP5, PEAQ-HT-AHSV1 VP3 and PEAQ-express AHSV-1 VP7 (1:1:1:1) or a combination of pEAQ-HT-AHSV-7 VP2, PEAQ-express AHSV-1 VP5, PEAQ-HT AHSV-1VP3 and pEAQ-express AHSV-1 VP7 (1:1:1:1), were harvested 8 days p.i and the clarified cellular lysates centrifuged through 70%-30% sucrose density gradients. The gradients were fractionated from the 55% sucrose layer to the 35% sucrose layer and 1/50 of each sucrose fraction assessed for the presence of AHSV-1/AHSV-7 capsid proteins via SDS-PAGE and immunoblotting with a guinea pig anti-AHSV-7 antiserum. Lane 1 contains the Precision Plus Protein™ Western C™ standard (Bio-Rad™) and the relevant sizes are indicated. Lanes 2-6 contain the 55%, 50%, 45%, 40%, 35% sucrose fractions, respectively, of the AHSV-1 sucrose gradient whilst lanes 7-11 contain 55%, 50%, 45%, 40%, 35% sucrose fractions, respectively, from the AHSV-1/AHSV-7 sucrose gradient. Lanes 12-15 contain 55%, 50%, 45%, 40% sucrose fractions proteins of pEAQ-HT cell lysate sucrose gradient, respectively, included as a negative control in this study. Arrows indicate the position of the AHSV-7 VP2 (123.6 kDa), AHSV-1 VP3 (103.2 kDa), AHSV-1 VP5 (56.6 kDa) and AHSV-1 VP7 (37.8 kDa) proteins on the immunoblot membrane.

FIG. 25: Immunoblot detection of AHSV-1 and/or AHSV-7 capsid proteins following sucrose density gradient centrifugation. Nicotiana benthamiana leaves, agroinfiltrated with PEAQ-express-AHSV1VP3-AHSV-1VP7 or a combination of pEAQ-HT-AHSV-1VP2, PEAQ-express-AHSV-1VP5, PEAQ-expressAHSV1VP3-AHSV-1VP7 (1:1:1), or a combination of pEAQ-HT-AHSV-7VP2, PEAQ-express-AHSV-1VP5, PEAQ-express-AHSV1VP3-AHSV-1VP7 (1:1:1) or a combination of PEAQ-HT-AHSV-7VP2, PEAQ-HT-AHSV-7VP5, PEAQ-express-AHSV1VP3-AHSV-1VP7 (1:1:1) were harvested 8 days p.i and the clarified cellular lysates centrifuged through 70%-30% sucrose density gradients. The gradients were fractionated from the 55% sucrose layer to the 35% sucrose layer and 1/50 of the 55-50% sucrose fractions assessed for the presence of AHSV-1/AHSV-7 capsid proteins via SDS-PAGE and immunoblotting with a guinea pig anti-AHSV-7 antiserum. Lane 1 contains the Precision Plus Protein™ Western C™ standard (Bio-Rad™) and the relevant sizes are indicated. Lanes 2-3 contain the 55%, 50% sucrose fractions, respectively, from the AHSV-1 CLP sucrose gradient. Lanes 4-5 contain the sucrose fractions, respectively, from the AHSV-1 VLP sucrose gradient. Lanes 6-7 contain the 55%, 50% sucrose fractions, respectively, from the single chimaeric AHSV-1/AHSV-7 VLP sucrose gradient. Lanes 8-9 contain the 55%, 50% sucrose fractions, respectively, from the double chimaeric AHSV-1/AHSV-7 VLP sucrose gradient. Arrows indicate the position of the AHSV-7 VP2 (123,6 kDa), AHSV-1 VP3 (103,2 kDa), AHSV-1 or AHSV-7 VP5 (56.6 kDa) and AHSV-1 VP7 (37.8 kDa) proteins on the immunoblot membrane.

FIG. 26: Transmission electron micrograph (TEM) images of sucrose-gradient purified (a) AHSV-1 VLPs, (b) AHSV-1 CLPs, (c) single chimaeric AHSV-1/AHSV-7 VLPs, (d) double chimaeric AHSV-1/AHSV-7 VLPs and (e)-(f) triple chimaeric AHSV-1/AHSV-3/AHSV-6 VLPs. Particles were visualized with a JEM-2100 Transmission electron microscope (JEOL™). Indicated with arrows are the virus-like particles (VLPs) and the core-like particles (CLPs).

FIG. 27: Plant produced BT VLPs detected in sucrose density gradient fractions 45-50% and separated by SDS-PAGE 4-12% Bolt™ precast gels. Capsid proteins are indicated with arrows and annotated. Lane 1, SeeBlue® Plus molecular marker; lanes 5 & 12, homogenous BTV-8 VLPs; lanes 2-4, 6, 7; BTV-3 double chimaeric; lane 7, BTV-3 single chimaeric; and lane 8 and 13, BTV-4 (only VP3 being BTV-8); lane 9, BTV-4 double chimaeric (BTV-4 VP2 and VP5, BTV-8 core); lane 10, BTV-4 single chimaeric (BTV-8 substituting only BTV-4 VP2).

FIG. 28: TEM analysis showing assembly of structural proteins into mixture of CLPs and VLPs. (A) BTV-8; (B) BTV-4 single chimaeric (substituting only with BTV-8 VP3); (C) BTV-4 double chimaeric (BTV-4 VP2 and VP5); (D) and € BTV-3 double chimaeric (BTV-3 VP2 and VP5) and (F) BTV-3 single chimaeric (BTV-8 core with BTV-3 VP2). Scale bars, 200 nm.

FIG. 29: Schematic representation of the constructs created for Agrobacterium-mediated expression of African horse sickness (AHSV) serotype 5 structural proteins in N. benthamiana and their resultant assembly into virus-like particles. (a) Stoichiometric diagram of virus-like particle formation. (b) Codon-optimized genes for AHSV-5 VP2, VP3, VP5 and VP7 were cloned into the pEAQ-HT plant expression vector (Sainsbury et al., 2009).

FIG. 30: Expression of recombinant AHSV-5 structural proteins and their assembly into virus-like particles in N. benthamiana. a) Western blot analysis of crude leaf extracts obtained 7 dpi with Agrobacterium radiobacter AGL1-ATCC BAA-101 containing pEAQ-AHS5 VP2 (lane 1), PEAQ-AHS5 VP3 (lane 2), PEAQ-AHS5 VP5 (lane 3), pEAQ-AHS5 VP7 (lane 4) or co-infiltrated with all 4 AHSV-5 recombinants (lane 5). Crude extract from leaves infiltrated with Agrobacterium transformed with PEAQ-HT expression vector lacking any goi, was used as a negative control (lane 6). Anti-AHSV 5 antiserum, which was unable to detect either VP3 or VP5, was used as the primary antibody. VP7 trimer (135 kDa), VP2 (123 kDa) and VP7 monomer (38 kDa) are indicated by arrow heads. Colour pre-stained protein standard, broad range (New England Biolabs™, Massachusetts, USA) indicated to the right of the blots was used as a molecular weight marker b) Fully assembled AHSV 5 virus-like particles imaged by TEM analysis of crude extracts from plants co-infiltrated with PEAQ-AHS5 VP2, pEAQ-AHS5 VP3, PEAQ-AHS5 VP5 and PEAQ-AHS5 VP7. Scale bar, 100 nm. c) N. benthamiana plant 7 dpi with all 4 AHSV-5 Agrobacterium recombinants.

FIG. 31: Purification of AHSV-5 VLPs by density gradient ultracentrifugation. Crude plant extracts from leaves co-infiltrated with all 4 AHSV-5 Agrobacterium recombinants, this using the mutated VP7 recombinant, were subjected to iodixanol density gradient ultra-centrifugation. a) Gradient fractions were collected from the bottom of the tube. b) Fractions 6 (lane 1), 7 (lane 2) and 8 (lane 3) were separated by denaturing SDS-PAGE followed by Coomassie blue staining. The location of the AHSV viral proteins VP2 (123 kD), VP3 (103 kD), VP5 (57 kD) and VP7mu (38 kD) are indicated to the right of the gel, while the molecular weight marker sizes are shown on the left. c) Gradient fraction 8 was imaged by TEM revealing the presence of fully assembled VLPs (white arrows) together with some assembly intermediates (yellow arrows). Scale bars, 50-200 nm.

FIG. 32: Immunogenicity of plant-produced AHSV-5 VLPs in guinea pigs. a) Vaccine- and control guinea pig groups (n=4) were vaccinated with plant-produced AHSV-5 VLPs (guinea pigs V2-V5) or PBS (guinea pigs C2-C5) respectively. Both vaccines were formulated with 5% Pet Gel A adjuvant (Seppic™, Paris, France). Guinea pigs V2-V5 were immunized with a dose of 16.5 μg AHSV-5 VLPs on day 0 and boosted with a dose of 50 μg VLPs on day 13, while guinea pigs C2-C5 were vaccinated with PBS per the same schedule. Serum was collected on day 41 and antibody responses were measured by standard ELISA. Absorbance values below 1:40 000 antiserum dilutions for guinea pigs in the vaccine group were too high to be measured. b) Antisera (1:10 000 dilution) from representative guinea pig V3 final bleed (lane 1) and pre-bleed (lane 2) were used to detect AHSV-5 VLPs in a standard Western blot analysis. The location of the AHSV viral proteins VP2, VP5 and VP7mu as well as the VP7mu trimer, are indicated to the left of the gel, while the molecular weight marker sizes are shown on the right. No signal was detected for the innermost core protein VP3.

FIG. 33: Plant produced BTV-8 and BTV-3 VLPs proteins (8 μg per lane) detected in lodixanol density gradient ultracentrifugation fractions 35-40% and separated by SDS-PAGE 4-12% Bolt™ precast gels. MW, SeeBlue® Plus2 Pre-stained Protein Standard. A) lanes 1-2, LBA4404 mediated BTV-8 VLPs; lanes 3-4, GV3101 pMP90 mediated BTV-8 VLPs; lanes 5-6, AGL-1 mediated BTV-8 VLPs; lanes 8-9, LBA4404 mediated BTV-3 VLPs; lanes 10-11, GV3101 pMP90 mediated BTV-3 VLPs and lanes 12-13, AGL-1 mediated BTV-3 VLPs. B) lanes 1-2, LBA4404 mediated BTV-3 VLPs; lanes 4-5, GV3101 pMP90 mediated BTV-3 VLPs and lanes 7-8, AGL-1 mediated BTV-3. VLPs Viral capsid protein 2 (VP2) is indicated with an arrow.

FIG. 34: TEM analysis of plant produced BTV-8 and BTV-3 VLPs subjected to lodixanol density gradient ultracentrifugation. A, LBA4404 mediated BTV-8 VLPs (64-73 nm); B, GV3101 pMP90 mediated BTV-8 VLPs (60-71 nm); C, AGL-1 mediated BTV-8 VLPs (64-80 nm); D, LBA4404 mediated BTV-3 VLPs (65-82 nm); E, GV3101 pMP90 mediated BTV-3 VLPs (59-78 nm) and F, AGL-1 mediated BTV-3 VLPs (60-80 nm). Scale bars, 200 nm.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

By “bluetongue” or “BT” is meant a virus belonging to a group of approximately 26 related but genetically distinct “serotypes”. The virus may also be referred to herein as “bluetongue virus” or “BTV”.

BTV is a double stranded ribonucleic acid (dsRNA) virus that causes an insect-borne, infectious non-contagious disease of both domesticated and wild ruminants; it is the type species of the genus Orbivirus that is classified into the family Reoviridae. Reoviridae is one of the largest families of viruses and includes major human pathogens, such as rotavirus, as well as pathogens of insects, reptiles, fish, plants and fungi. Orbiviruses differ from other members of the Reoviridae family in that they can multiply in both arthropod and vertebrate cells, causing severe disease and high mortality. BTV is transmitted between its hosts by Culicoides spp., causing disease in ruminants worldwide.

Virus protein (VP) 2 is the most variable of the BTV capsid proteins and contains the epitopes involved in virus neutralisation and serotype determination (DeMaula et al., 2000, Huismans and Erasmus, 1981). Twenty six distinct serotypes of BTV have been identified based on neutralisation activity of VP2 as well as with BTV specific real time reverse transcriptase polymerase chain reaction (RT-PCR). Each serotype shows variation that is associated with the geographical origins of the virus from around the world. Molecular studies on BTV isolates from different geographic regions have further divided BTV into two major topotypes, namely the eastern and western lineages.

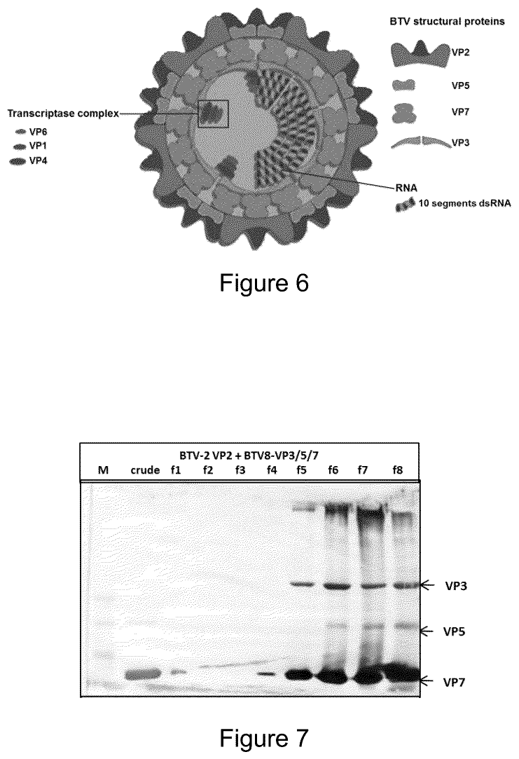

The BTV genome is a double-stranded circular dsRNA surrounded by a protein capsid. BTV can replicate in both wild and domestic ruminants as well as some species of deer. Replication takes place in both the host and the Culicoides insect vector. BTV virions are complex three-layered icosahedral structures that are ˜80 nanometer (nm) in diameter. The virions are composed of a core of ten segments of dsRNA encapsulated by seven structural proteins (four major and three minor proteins) that are arranged into three distinct layers (FIG. 6).

The three minor proteins (viral protein (VP) 1, VP4 and VP6) are enclosed by the subcore that is made up of VP3. The core-surface layer consists of VP7. The outer capsid is composed of major proteins VP2 and VP5 which are laid onto the foundation provided by the core. The minor proteins together with the genomic RNA form the virus replication complex, whereas the four major proteins make up the capsid of the virus. In addition to the structural proteins BTV has four non-structural (NS) proteins (NS1, NS2, NS3/3a and NS4) which are involved in virus replication and assembly in BTV-infected cells.

The chimaeric VLPs and compositions according to the invention may be used to treat or prevent BTV infection or conditions associated with BTV infection. By “condition associated with BTV infection” is meant any condition, disease or disorder that has been correlated with the presence of an existing BTV infection, includes secondary effects, such as reductions in milk production, weight gain, wool break and temporary infertility.

BTV can infect all known species of domestic and wild ruminants. Severe disease usually occurs in the fine-wool and mutton breeds of sheep as well as some species of deer. BTV infection of cattle, goats and wild ruminant species is mostly asymptomatic or subclinical. In BTV endemic areas BTV-infected sheep develop only mild or no obvious disease. The bluetongue after which the disease is named is seen only in serious clinical cases.

Onset of the disease in sheep is typically characterised by high fever lasting 5-7 days. Clinical signs of disease can include fever, depression, excessive salivation, nasal discharge, facial oedema, hyperaemia and ulceration of the oral mucosa, coronitis, lameness and death. Abortion can occur in pregnant animals as well as teratogenic defects in calves. The severity of clinical disease and mortality rate is influenced by the breed and age of the animal as well as the virus strain that causes the infection. In acute cases of BT, clinical signs in sheep are mainly associated with damage to microvascular endothelial cells.

After recovery from BT animals may suffer from a number of long-lasting secondary effects, such as reductions in milk production, weight gain, wool brake and temporary infertility.

Pathogenesis of BTV infection is similar in sheep and cattle as well as other species of ruminants. After an animal gets infected with BTV, through the bite of a Culicoides vector, the virus will travel to the regional lymph node where initial replication takes place. The virus then spreads throughout the body to a variety of tissues, where replication occurs mainly in mononuclear phagocytic and endothelial cells.

Viraemia is cell associated and can be prolonged in domestic ruminants. During viraemia BTV is associated with all blood cells, but late in the course of infection the virus is mostly associated with the erythrocytes. The longer lifespan of erythrocytes facilitates prolonged infection of ruminants, as well as the infection of the haematophagous insect vectors that feed on viraemic ruminants. Infectious virus can co-circulate for several weeks with high neutralising antibody titres, the maximum period of viraemia in sheep is about 50 days and in cattle about 100 days.

By “African horse sickness” or “AHS” is meant the disease itself. The virus is referred to herein as “African horse sickness virus” or “AHSV” belongs to a group of approximately 9 related but genetically distinct “serotypes”.

AHSV is a double stranded ribonucleic acid (dsRNA) virus that causes an infectious, non-contagious disease of equids. It is classified as an Orbivirus in the family Reoviridae. The virus is transmitted by biting midges of the Culicoides species.

The AHS virion is an icosahedral, non-enveloped particle, composed of three concentric layers surrounding the segmented double-stranded RNA genome. The AHS virion has been reported to be between 70 nm-87 nm in diameter. The subcore, composed of structural protein VP3, encloses 10 linear genome segments and enzymatic minor proteins VP1, VP3 and VP6. The subcore is covered by a layer of VP7 trimers forming the core particle. The core is surrounded by the outermost layer composed of structural proteins VP5 and VP2, with VP2 being the neutralizing antigen and serotype determinant. There are nine known serotypes of AHSV and all are present within South Africa and most parts of sub-Saharan Africa.

The chimaeric VLPs and compositions according to the invention may be used to treat or prevent AHSV infection or conditions associated with AHSV infection. By “condition associated with AHSV infection” is meant any condition, disease or disorder that has been correlated with the presence of an existing AHSV infection and includes secondary effects.

AHSV infects equid species, such as horses, donkeys, mules and zebra, amongst others. The mortality rate in horses, the most susceptible species, can be up to 95% while donkeys and mules generally develop milder disease. Zebras are considered the natural vertebrate host of AHSV and rarely exhibit clinical signs of infection. Respiratory and circulatory functions are impaired in diseased animals and result in oedema of subcutaneous and intermuscular tissues, of lungs and haemorrahages of serosal surfaces. These animals also exhibit pyrexia and loss of appetite.

A compound according to the invention includes, without limitation, a single chimaeric Orbivirus VLP including a core comprising capsid proteins VP3, VP5 and VP7 from one serotype of an Orbivirus species and an outer layer comprising a VP2 selected from any one of the other serotypes of the same Orbivirus species. In an alternative embodiment a compound of the invention includes, without limitation, a double chimaeric Orbivirus VLP including a core comprising capsid proteins VP3 and VP7 from one serotype of an Orbivirus species and an outer layer comprising the VP2 and VP5 capsid proteins selected from any one of the other serotypes of the same Orbivirus species.

When the Orbivirus species is BTV, a compound according to the invention includes, without limitation, a single chimaeric VLP including a core comprising, for instance, BTV-8 capsid proteins VP3, VP5 and VP7 and an outer layer comprising a BTV VP2 selected from any one of the 26 BTV serotypes, with the exception of BTV-8. In an alternative embodiment a compound of the invention includes, without limitation, a double chimaeric VLP including a core comprising, for instance, BTV-8 VP3 and VP7 capsid proteins and an outer layer comprising BTV VP2 and VP5 capsid proteins selected from any one of the 26 BTV serotypes, with the exception of BTV-8

Similarly, when the Orbivirus species is AHSV, a compound according to the invention includes, without limitation, a single chimaeric VLP including a core comprising, for instance, AHSV-1 capsid proteins VP3, VP5 and VP7 and an outer layer comprising AHS VP2 selected from any one of the 8 remaining AHSV serotypes. In an alternative embodiment a compound of the invention includes, without limitation, a double chimaeric VLP including a core comprising, for instance, AHSV-1 VP3 and VP7 capsid proteins and an outer layer comprising AHSV VP2 and VP5 capsid proteins selected from any one of the remaining 8 AHSV serotypes.

It will be appreciated by those of skill in the art that the Orbivirus species could be an Orbivirus selected from the group consisting of Lebombo virus (LEBV), Pata virus (PATAV), African horse sickness virus (AHSV), Bluetongue virus (BTV), Altamira virus (ALTV), Almeirim virus (AMRV), Caninde virus (CANV), Changuinola virus (CGLV), Irituia virus (IRIV), Jamanxi virus (JAMV), Jari virus (JARIV), Gurupi virus (GURV), Monte Dourado virus (MDOV), Ourem virus (OURV), Purus virus (PURV), Saraca virus (SRAV), Acado virus (ACDV), Corriparta virus (CORV), Eubenangee virus (EUBV), Ngoupe virus (NGOV), Tilligerry virus (TILV), Epizootic hemorrhagic disease virus (EHDV), Kawanabe virus, Equine encephalosis virus (EEV), Great Island virus, Kemerovo virus (KEMV), Essaouira virus (ESSV), Kala iris virus (KIRV), Mill Door/79 virus (MILDV), Rabbit syncytium virus (RSV), Tribeč virus (TRBV), Broadhaven virus (BRDV), Orungo virus (ORUV), Abadina virus (ABAV), Apies River virus, Bunyip Creek virus (BCV), Chuzan (Kasba) virus (SBV), CSIRO Village virus (CVGV), D'Aguilar virus (DAGV), Marrakai virus (MARV), Petevo virus (PETV), Vellore virus (VELV), Llano Seco virus (LLSV), Minnal virus (MINV), Netivot virus (NETV), Umatilla virus (UMAV), Wallal virus (WALV) and Mitchell River virus (MRV).

A “protein,” “peptide” or “polypeptide” is any chain of two or more amino acids, including naturally occurring or non-naturally occurring amino acids or amino acid analogues, irrespective of post-translational modification (e.g., glycosylation or phosphorylation).

The terms “nucleic acid” or “nucleic acid molecule” encompass both ribonucelotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides.

The term “complementary” refers to two nucleic acids molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

In some embodiments, a chimaeric VLP of the invention may include, without limitation, BTV-8 VP3 (SEQ ID NO: 1 or 2), VP5 (SEQ ID NO: 3 or 4) and VP7 (SEQ ID NO: 5 or 6) polypeptides or derivatives thereof and/or a VP2 polypeptide selected from the group consisting of SEQ ID NOs 7 to 11, or derivatives thereof. Another embodiment of the invention includes, without limitation, nucleic acid molecules encoding the aforementioned amino acid sequences. It will however be appreciated by those of skill in the art that the VP3, VP5 and VP7 polypeptides may be polypeptides from any of the BTV serotypes, for example BTV-3 VP5 (SEQ ID NO: 12), BTV-4 VP5 (SEQ ID NO: 13) and BTV-4 VP7 (SEQ ID NO:14).

In other embodiments, a chimaeric VLP of the invention may include, without limitation, AHSV-1 VP3, VP5 and VP7 polypeptides having an amino acid sequence of SEQ ID NOs: 15, 16 and 17, respectively or derivatives thereof and/or a VP2 polypeptide of SEQ ID NO: 18 or 19, or derivatives thereof. Another embodiment of the invention includes, without limitation, nucleic acid molecules encoding the aforementioned amino acid sequences. It will however also be appreciated by those of skill in the art that the VP3, VP5 and VP7 polypeptides may be polypeptides from any of the AHSV serotypes, for example AHSV-7 VP5 (SEQ ID NO:20).

As used herein a “substantially identical” sequence is an amino acid or nucleotide sequence that differs from a reference sequence only by one or more conservative substitutions, or by one or more non-conservative substitutions, deletions, or insertions located at positions of the sequence that do not destroy or substantially reduce the antigenicity of one or more of the expressed polypeptides or of the polypeptides encoded by the nucleic acid molecules. Alignment for purposes of determining percent sequence identity can be achieved in various ways that are within the knowledge of those with skill in the art. These include using, for instance, computer software such as ALIGN, Megalign™ (DNASTAR®), CLUSTALW or BLAST® software. Those skilled in the art can readily determine appropriate parameters for measuring alignment, including any algorithms needed to achieve maximal alignment over the full length of the sequences being compared. In one embodiment of the invention there is provided for a polypeptide or polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

In an alternative embodiment of the invention, the chimaeric VLPs may be prepared by, for instance, inserting, deleting or replacing amino acid residues at any position of the BTV VP2, VP3, VP5 or VP7 polypeptide sequences and/or, for instance inserting, deleting or replacing nucleic acids at any position of the nucleic acid molecule encoding the BTV VP2, VP3, VP5 or VP7 polypeptides.

In an alternative embodiment of the invention, the chimaeric VLPs may be prepared by, for instance, inserting, deleting or replacing amino acid residues at any position of the AHSV VP2, VP3, VP5 or VP7 polypeptide sequences and/or, for instance inserting, deleting or replacing nucleic acids at any position of the nucleic acid molecule encoding the AHSV VP2, VP3, VP5 or VP7 polypeptides.

Those skilled in the art will appreciate that polypeptides, peptides or peptide analogues can be synthesised using standard chemical techniques, for instance, by automated synthesis using solution or solid phase synthesis methodology. Automated peptide synthesisers are commercially available and use techniques known in the art. Polypeptides, peptides and peptide analogues can also be prepared from their corresponding nucleic acid molecules using recombinant DNA technology.

In some embodiments, the nucleic acid molecules of the invention may be operably linked to other sequences. By “operably linked” is meant that the nucleic acid molecules encoding the VP2, VP3, VP5 and/or VP7 polypeptides of the invention and regulatory sequences are connected in such a way as to permit expression of the proteins when the appropriate molecules are bound to the regulatory sequences. Such operably linked sequences may be contained in vectors or expression constructs which can be transformed or transfected into host cells for expression. It will be appreciated that any vector or vectors can be used for the purposes of expressing the VP2, VP3, VP5 and/or VP7 of the invention.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or gene sequences can be introduced into a cell. There are various types of vectors known in the art including plasmids, viruses, bacteriophages and cosmids. Generally polynucleotides or gene sequences are introduced into a vector by means of a cassette. The term “cassette” refers to a polynucleotide or gene sequence that is expressed from a vector, for example, the polynucleotide or gene sequences encoding the VP2, VP3, VP5 and/or VP7 polypeptides of the invention. A cassette generally comprises a gene sequence inserted into a vector, which in some embodiments, provides regulatory sequences for expressing the polynucleotide or gene sequences. In other embodiments, the vector provides the regulatory sequences for the expression of the VP2, VP3, VP5 and/or VP7 polypeptides. In further embodiments, the vector provides some regulatory sequences and the nucleotide or gene sequence provides other regulatory sequences. “Regulatory sequences” include but are not limited to promoters, transcription termination sequences, enhancers, splice acceptors, donor sequences, introns, ribosome binding sequences, poly(A) addition sequences, and/or origins of replication.

The chimaeric VLPs or compositions of the invention can be provided either alone or in combination with other compounds (for example, nucleic acid molecules, small molecules, peptides, or peptide analogues), in the presence of an adjuvant, or any carrier, such as a pharmaceutically acceptable carrier and in a form suitable for administration to mammals, for example, humans, cattle, sheep, etc.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the chimaeric VLPs or vaccine composition to a subject. The pharmaceutically acceptable carrier can be suitable for intramuscular, intraperitoneal, intravenous, subcutaneous, oral or sublingual administration. Pharmaceutically acceptable carriers include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

Suitable formulations or compositions to administer the chimaeric VLPs and compositions to subjects who are to be prophylactically treated for an Orbivirus infection, who are suffering from an Orbivirus infection or subjects which are presymptomatic for a condition associated with Orbivirus infection fall within the scope of the invention. Any appropriate route of administration may be employed, such as, parenteral, intravenous, subcutaneous, intramuscular, intracranial, intraorbital, ophthalmic, intraventricular, intracapsular, intraspinal, intrathecal, intracistemal, intraperitoneal, intranasal, aerosol, topical, or oral administration.

As used herein the term “subject” includes wild and domestic ruminants, equids or any specified target animal

For vaccine formulations, an effective amount of the chimaeric VLPs or compositions of the invention can be provided, either alone or in combination with other compounds, with immunological adjuvants, for example, aluminium hydroxide dimethyldioctadecylammonium hydroxide or Freund's incomplete adjuvant. The chimaeric VLPs or compositions of the invention may also be linked with suitable carriers and/or other molecules, such as bovine serum albumin or keyhole limpet hemocyanin in order to enhance immunogenicity.

In some embodiments, the chimaeric VLPs or compositions according to the invention may be provided in a kit, optionally with a carrier and/or an adjuvant, together with instructions for use.

An “effective amount” of a compound according to the invention includes a therapeutically effective amount, immunologically effective amount, or a prophylactically effective amount. A “therapeutically effective amount” refers to an amount effective, at dosages and for periods of time necessary, to achieve the desired therapeutic result, such as treatment of an Orbivirus infection or a condition associated with such infection. The outcome of the treatment may for example be measured by a decrease in viremia, inhibition of viral gene expression, delay in development of a pathology associated with the Orbivirus infection, stimulation of the immune system, or any other method of determining a therapeutic benefit. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

The dosage of any of the chimaeric VLPs or compositions of the present invention will vary depending on the symptoms, age and body weight of the subject, the nature and severity of the disorder to be treated or prevented, the route of administration, the Orbivirus infection being treated and the form of the composition. Any of the compositions of the invention may be administered in a single dose or in multiple doses. The dosages of the compositions of the invention may be readily determined by techniques known to those of skill in the art or as taught herein.

By “immunogenically effective amount” is meant an amount effective, at dosages and for periods of time necessary, to achieve a desired immune response. The desired immune response may include stimulation or elicitation of an immune response, for instance a T or B cell response.

A “prophylactically effective amount” refers to an amount effective, at dosages and for periods of time necessary, to achieve a desired prophylactic result, such as prevention of onset of a condition associated with an Orbivirus infection. Typically, a prophylactic dose is used in subjects prior to or at an earlier stage of disease, so that a prophylactically effective amount may be less than a therapeutically effective amount.

Dosage values may vary with the severity of the condition to be alleviated. For any particular subject, specific dosage regimens may be adjusted over time according to the individual need and the judgment of the person administering or supervising the administration of the chimaeric VLPs or compositions of the invention. Dosage ranges set forth herein are exemplary only and do not limit the dosage ranges that may be selected. The amount of active compound(s) in the composition may vary according to factors such as the disease state, age, sex, and weight of the individual. Dosage regimens may be adjusted to provide the optimum therapeutic response. For example, a single dose may be administered, or multiple doses may be administered over time. It may be advantageous to formulate the compositions in dosage unit forms for ease of administration and uniformity of dosage.

The term “preventing”, when used in relation to an infectious disease, or other medical disease or condition, is well understood in the art, and includes administration of a composition which reduces the frequency of or delays the onset of symptoms of a condition in a subject relative to a subject which does not receive the composition. Prevention of a disease includes, for example, reducing the number of diagnoses of the infection in a treated population versus an untreated control population, and/or delaying the onset of symptoms of the infection in a treated population versus an untreated control population.

The term “prophylactic or therapeutic” treatment is well known to those of skill in the art and includes administration to a subject of one or more of the compositions of the invention. If the composition is administered prior to clinical manifestation of the unwanted condition (e.g., disease or other unwanted state of the subject) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

Toxicity and therapeutic efficacy of compositions of the invention may be determined by standard pharmaceutical procedures in cell culture or using experimental animals, such as by determining the LD₅₀ and the ED₅₀. Data obtained from the cell cultures and/or animal studies may be used to formulating a dosage range for use in a subject. The dosage of any composition of the invention lies preferably within a range of circulating concentrations that include the ED₅₀ but which has little or no toxicity. The dosage may vary within this range depending upon the dosage form employed and the route of administration utilized. For compositions of the present invention, the therapeutically effective dose may be estimated initially from cell culture assays.

Provided herein are methods for producing a chimaeric BTV VLP in a plant cell, which comprises a core comprising BTV-8 capsid proteins VP3, VP5 and VP7 and an outer layer comprising BTV VP2 proteins selected from any one of the 26 BTV serotypes and methods for producing a chimaeric BTV VLP in a plant cell, which comprises a core comprising BTV-8 VP3 and VP7 capsid proteins and an outer layer comprising BTV VP2 and VP5 capsid proteins selected from any one of the 26 BTV serotypes.

Similarly, methods for producing a chimaeric AHSV VLP in a plant cell, which comprises a core comprising AHSV-1 capsid proteins VP3, VP5 and VP7 and an outer layer comprising VP2 protein selected from any one of the 8 remaining AHSV serotypes and methods for producing a chimaeric AHSV VLP in a plant cell, which comprises a core comprising AHSV-1 VP3 and VP7 capsid proteins and an outer layer comprising AHSV VP2 and VP5 capsid proteins selected from any one of the remaining 8 AHSV serotypes.

A “VLP” or “virus-like particle” refers to the capsid-like structure which results from the assembly of Orbivirus VP2, VP3, VP5 and VP7 polypeptides. These particles are antigenically and morphologically similar to native Orbivirus virus virions but do not include viral genetic material; accordingly, these particles are not replicating nor infectious.

The invention also relates in part to a method of eliciting an immune response in a subject comprising administering to a subject in need thereof a prophylactically effective amount of the chimaeric VLPs or compositions of the present invention.

The following examples are offered by way of illustration and not by way of limitation.

### Example 1

Nicotiana sp. codon-optimised BTV-8 VP2 (SEQ ID NO:27), VP3 (SEQ ID NO:21), VP5 (SEQ ID NO:23) and VP7 (SEQ ID NO:25) were synthesised (Geneart, Germany). The plant codon optimised nucleotide sequences encode the following proteins: BTV-8 VP2 (SEQ ID NO:7), VP3 (SEQ ID NO:1), VP5 (SEQ ID NO:3) and VP7 (SEQ ID NO:5). Primers were designed to add restriction enzyme sites (AgeI and XhoI) on the 5′ and 3′ termini, respectively (Table 1) such that they could be cloned into the pEAQ-HT expression vector using these sites.

TABLE 1
BTV gene specific primers.

| Pro- | Primer | | SEQ ID |
|---|---|---|---|
| tein | Name | Sequence | NO. |
| BTV-8 | pEAQ- | 5′ GCACCGGTATGGAAGAAC | (SEQ ID |
| VP2 | HTVP2F | TCGCTATCCCAA 3′ | NO: 41) |
| | cVP2coR | 5′ GCCTCGAGTCAAACGTTG | (SEQ ID |
| | | AGGAGCTTAGTAAG 3′ | NO: 42) |
| BTV-8 | pEAQ- | 5′ GCACCGGTATGGCTGCTC | (SEQ ID |
| VP3 | HTVP3F | AAAATGAGCAAAG 3′ | NO: 43) |
| | cVP3coR | 5′ GCCTCGAGTTAAACAGTT | (SEQ ID |
| | | GGAGCAGCAAGC 3′ | NO: 44) |
| BTV-8 | pEAQ- | 5′ GCACCGGTATGGGAAAGA | (SEQ ID |
| VP5 | HTVP5F | TTATTAAGTCCCTCTC 3′ | NO: 45) |
| | cVP5coR | 5′ GCCTCGAGTCAAGCGTTC | (SEQ ID |
| | | CTAAGGAAGAG 3′ | NO: 46) |
| BTV-8 | pEAQ- | 5′ GCACCGGTATGGATACAA | (SEQ ID |
| VP7 | HTVP7F | TTGCTGCTAGGG 3′ | NO: 47) |
| | cVP7coR | 5′ GCCTCGAGTCACACATAA | (SEQ ID |
| | | GCAGCCCTAG 3′ | NO: 48) |

Resulting constructs were named PEAQ-HT-VP2, PEAQ-HT-VP3, PEAQ-HT-VP5 and PEAQ-HT-VP7. These constructs were sequenced and transformed into A. tumefaciens LBA4404.

Ten ml cultures of all four recombinant constructs were grown up in LB containing magnesium sulphate (2 mM), rifampicin (50 μg/ml) and kanamycin (30 μg/ml) at 27° C. overnight with agitation at 200 rpm. A 10th of the volume was transferred to induction medium (LB, 10 mM MES, pH 5.6) containing the same concentration of antibiotics as well as 20 μM acetosyringone. These were incubated overnight at 27° C. with agitation at 200 rpm and then centrifuged at 4000 rpm to pellet the cells. The cell pellets were resuspended in 5 ml of infiltration medium (10 mM MES, 10 mM MgCl₂, 3% sucrose, pH 5.6) supplemented with 200 μM acetosyringone and incubated at room temperature for 2 h. The OD₆₀₀ of each culture was measured and the cultures diluted to an OD of 1.8 in infiltration medium. They were then combined in a ratio of 1:1:2:1 (VP2:VP3:VP5:VP7) and syringe-infiltrated into the abaxial surfaces of six-week-old N. benthamiana plants.

At 9 days post infiltration (dpi) the leaves were ground up and immediately cut up into fine pieces and homogenized in three volumes of ice cold bicine buffer (50 mM bicine (pH 8.4), 20 mM sodium chloride (NaCl), and 1× Complete Mini, EDTA-free protease inhibitor cocktail (Roche™)) lacking NLS and DTT. The homogenate was clarified by centrifugation at 1000×g for 10 min after which the supernatant was filtered through four layers of Miracloth™ (Merck™). The crude plant sap was overlayed onto 5 ml of a 40% iodixanol (Optiprep™, Sigma-Aldrich™) cushion prepared in 50 mM Tris-HCl, pH 8.4 and 20 mM NaCl after which it was centrifuged for 2 h at 79 000×g in a SW 32 Ti rotor (Beckman™). The 40% iodixanol cushion was collected after centrifugation from the bottom of the tube and overlayed onto 5 ml of a 20% to 60% step gradient (1 ml of each gradient in 10% incrementing steps) and centrifuged as above. Fractions of 0.5 ml were collected from the bottom of the tubes and analyzed by western blotting and TEM.

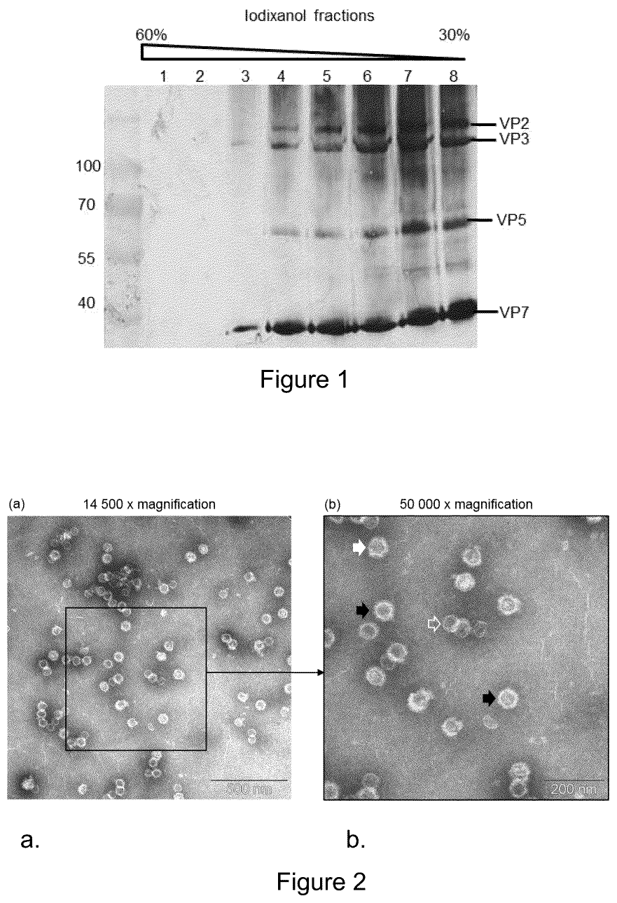

For western blot analysis, the iodixanol fractions were incubated at 90° C. for 10 min in loading buffer. The proteins were separated on 8% SDS polyacrylamide gels where equal amounts of total protein were loaded in each lane. After electrophoresis the proteins were transferred onto nitrocellulose membranes using a Trans-blot® SD semi-dry transfer cell (Bio-Rad™). Membranes were probed with a 1:2000 dilution of BTV-8 sheep serum (Thuenemann et al., 2013) and subsequently with a 1:10000 dilution of anti-goat/sheep alkaline phosphatase-conjugated secondary antibody (Sigma-Aldrich™). Detection was performed with 5-bromo-4-chloro-3-indoxyl-phosphate (BCIP) and nitroblue tetrazolium (NBT) phosphatase substrate (BCIP/NBT 1-component, KPL™). Western blot analysis of the first 8 fractions collected from the iodixanol gradient after centrifugation showed the presence of all four bands constituting the BTV-8 VLPs in fractions 4 (approximately 40%-50% iodixanol) up until fraction 8 (20%-30% iodixanol) (FIG. 1). These samples were also resolved on a Coomassie-stained SDS-PA gel. Only VP3, VP5 and VP7 could be observed on the gel. TEM analysis on fractions 5 to 8 showed only the presence of CLPs in the samples. These results indicate that VP2 and VP5 are not assembling with the CLPs to form VLPs, but that they are being co-purified with the particles.

For TEM, copper grids (mesh size 200) were floated for 2 min on a 1:200 dilution of BTV-8 sheep serum and washed twice with sterile water. Thereafter the grids were floated on a 1:10 dilution of crude plant extract for 5 min and washed three times with sterile water. The samples were negatively stained for 1 min with 2% uranyl acetate. Fractionated samples from the density gradients were treated similarly except they were not captured onto the grids with anti-BTV-8 sheep serum. All grids were viewed using a Technai™ G2 TEM. TEM of fraction 4 from the density gradient showed a mixed population of both CLPs and VLPs based on diameter measurements (FIG. 2a and b): CLPs measured 60 to 69 nm in diameter and VLPs measured 72 to 80 nm in diameter. Ten fields of view, at a magnification of 14 500 ×, showed approximately 80 particles in each view of which approximately 10% were VLPs (FIG. 2a). FIG. 2b shows more detail of the particles, at 50 000× magnification, making it easier to distinguish CLPs (single shelled particles) from VLPs (double shelled particles).

TEM on samples from leaves co-infiltrated with BTV VP constructs was also carried out. The BTV-8 PEAQ-HT-VP2, PEAQ-HT-VP3, PEAQ-HT-VP5 and PEAQ-HT-VP7 constructs were cultured and combined (as described previously) in a ratio of 1:1:2:1 (VP2:VP3:VP5:VP7) and syringe-infiltrated into the abaxial surfaces of six-week-old N. benthamiana plants.

At 9 dpi a whole leaf was picked from the infiltrated plant and a 3 cm×3 cm piece was cut out with a scalpel blade in the presence of 2.5% gluteraldehyde (25% gluteraldehyde diluted in 0.1 M phosphate buffer (pH 7.4)). The leaf sample was soaked in 2.5% gluteraldehyde for 6 hours after which it was cut into 1 mm×3 mm fragments, also in the presence of 2.5% gluteraldehyde. The leaf fragments were left in 2.5% gluteraldehyde overnight at 4° C. The following morning the leaf fragments were washed 3 times, 5 minutes for each wash, in 0.1 M phosphate buffer (pH 7.4). The leaf fragments were fixed for one hour in one part 2% osmium tetroxide and one part 0.2 M phosphate buffer (pH 7.4) after which it was washed twice for 5 minutes each with 0.1 M phosphate buffer (pH 7.4) followed with two washes of 5 min each with water.

After washing the leaf fragments were sequentially dehydrated. The leaf fragments were incubated for 5 minutes each in 30%, 50%, 70%, 80%, 90% and 95% ethanol. The fragments were incubated for 10 minutes in 100% ethanol; this step was repeated twice. After the ethanol dehydrations series the leaf fragments were further dehydrated by 10 minute incubation in 100% acetone, repeated twice. The leaf fragments were mixed overnight in 1:1 acetone: Spurr's resin.

The following day half of the 1:1 acetone: Spurr's resin mixture was removed (after centrifugation) and replaced with 100% Spurr's resin to yield a 1:3 acetone: Spurr's resin mixture. The sample was mixed for four hours at room temperature, after which the acetone/resin mixture was removed and replaced with 100% Spurr's resin. The leaf fragments were incubated in 100% Spurr's resin for three days at 4° C. The 100% Spurr's resin was replaced with fresh resin and incubated for four hours at room temperature after which the resin was replaced again and incubated overnight at room temperature. The following morning the samples were embedded and incubated for 24 hours at 60° C.

The embedded leaf samples were cut into ultrathin sections with a diamond knife and collected onto copper grids. The copper grids were stained with uranyl acetate for 10 minutes after which they were washed five times, 15 seconds each, with water. The grids were blotted dry and transferred to lead citrate for 10 minutes after which the grids were washed with water and blotted dry. Grids were viewed using the Technai™ G2 transmission electron microscope.

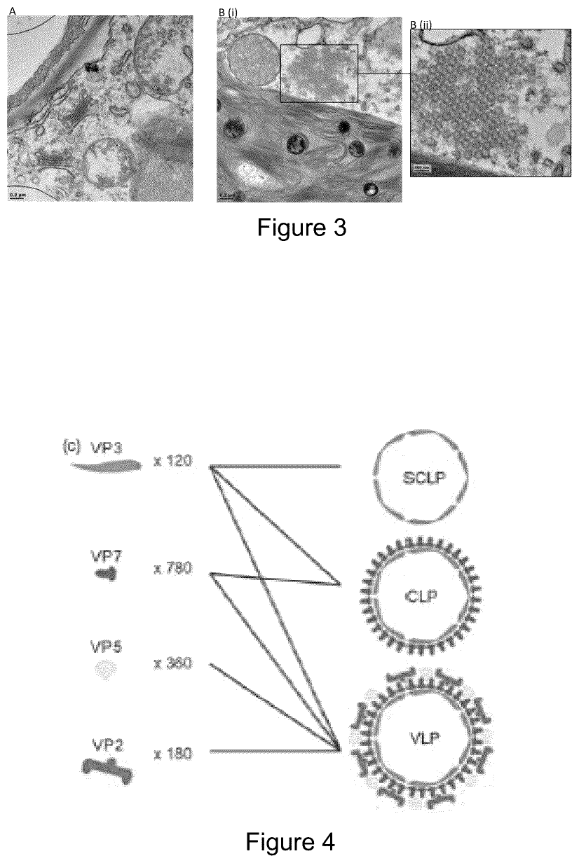

FIG. 3A shows a leaf section infiltrated only with infiltration medium as a negative control. TEM of the leaf sections (FIG. 3B (i) and (ii)) showed particles ranging in size from 60 to 78 nm in diameter, indicating that the mixed population consists of both CLPs and VLPs. The particles are present in the cytoplasm of the plant cell and are arranged in arrays.

### Example 2

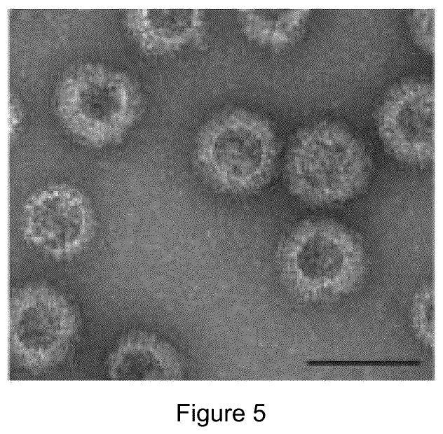

In Example 1 the present inventors investigated the transient production of BTV VLPs in plants as an alternative cheaper source of safe and effective vaccine. The inventors have successfully shown that co-expression of Bluetongue virus (BTV) serotype 8 VP2, VP3, VP5 and VP7 capsid encoding genes by Agrobacterium-mediated infiltration of N. benthamiana results in the efficient assembly of virus-like particles (VLPs), and that these VLPs are highly immunogenic and are protective in sheep (FIG. 5).

The present example was performed to demonstrate that it is possible to produce BTV VLPs covering a wide range of serotypes by using the pre-existing BTV 8 VP3, 5 and 7 proteins as a common scaffold or core on which different serotype-specific VP2s could be presented representing other BTV serotypes thus producing a multivalent antigen.

To prove this concept, we tested the production of VLPs in plants with a different BTV VP2 serotype i.e. a BTV-2 VP2. The VP2 gene was codon-optimised for N. benthamiana (SEQ ID NO:28) and synthesised by GenScript™, cloned into the plant expression vector pEAQ-HT (Sainsbury et al., 2009) and electroporated into Agrobacterium tumefaciens LBA4404. This recombinant strain as well as those encoding BTV serotype 8 VP3, 5 and 7 genes (made previously) which are required for VLP assembly were co-infiltrated into N. benthamiana and the leaves were screened for the presence of all 4 proteins by western blotting after 8 days. A preliminary western blot was carried out on the samples to determine the presence of BTV VP proteins. VP3, 5 and 7 proteins were detected but VP2 was not (data not shown). The antiserum used for this western blot is polyclonal serum from sheep which have been injected with plant-produced BTV-8 VLPs. It is possible that the VP2 is not detected by this antiserum in this western blot because it is serotype 8-specific.

We continued with scaling up of BTV VP production so that sufficient material could be obtained for purification and TEM analysis. Thirty plants were co-infiltrated with cultures of the 4 different recombinant strains and harvested after 8 days.

The leaf material was homogenised, centrifuged to get rid of particulate matter, and the supernatant filtered through Miracloth™. The filtrate was then loaded on top of a 30% Optiprep™ cushion made up in bicine buffer. The tubes were centrifuged at 22 000 rpm for 2 hours in a SW32Ti rotor and the interface between the cushion and supernatant aspirated. This was loaded on top of a 20 to 60% Optiprep™ gradient (made up in bicine buffer) and centrifuged at 22 000 rpm for 2 h in a SW32Ti rotor. The tube was fractionated into 10×1 ml fractions and some of the fractions were analysed on a western blot using the same polyclonal sheep antiserum used above to detect BTV VP protein.

FIG. 7 shows the western blot where BTV-8 VP3, VP5 and VP7 are visible as 102 kDa, 59 kDa and 38 kDa sized proteins. BTV-2 VP2 (expected to be 111 kDa) is not visible, but this is not unexpected as the antiserum is serotype 8-specific.

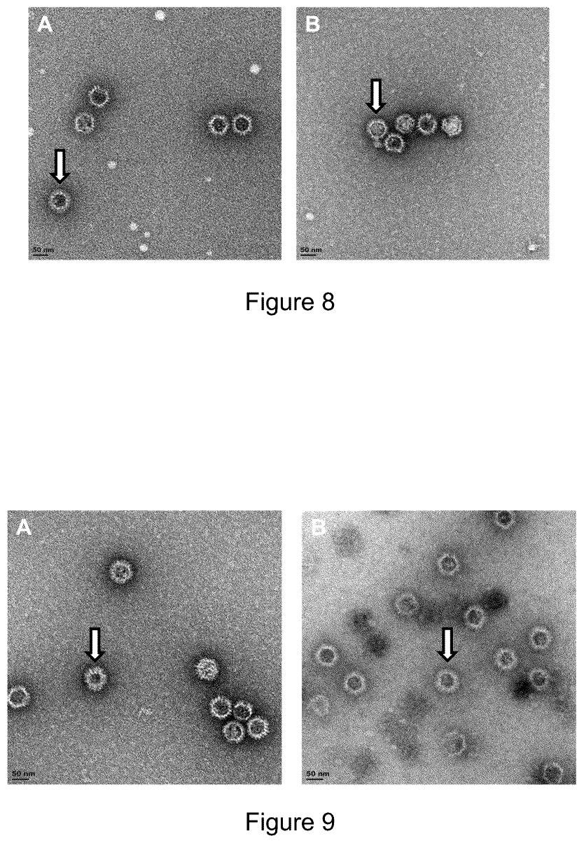

Fraction 5 of the gradient was analysed by transmission electron microscopy (TEM). FIGS. 8 and 9 show examples of the particles that were produced. It seems that although there were some VLPs observed (distinguished by a thicker outer ring), more than half of the particles purified consisted of subcore-like and core-like particles. However, the presence of some VLPs does indicate that the formation of chimaeric BTV particles is possible.

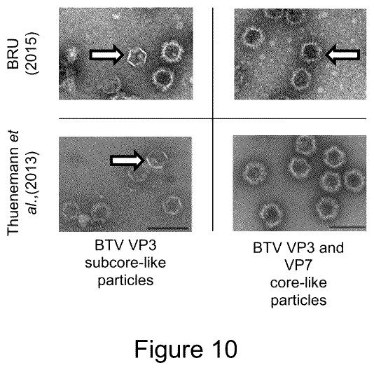

We have shown that the co-infiltration of only BTV-8 VP3- and VP7-encoding constructs results in the formation of core-like particles (FIG. 10) indicating that the core is being formed on which VP5 and VP2 can bind.

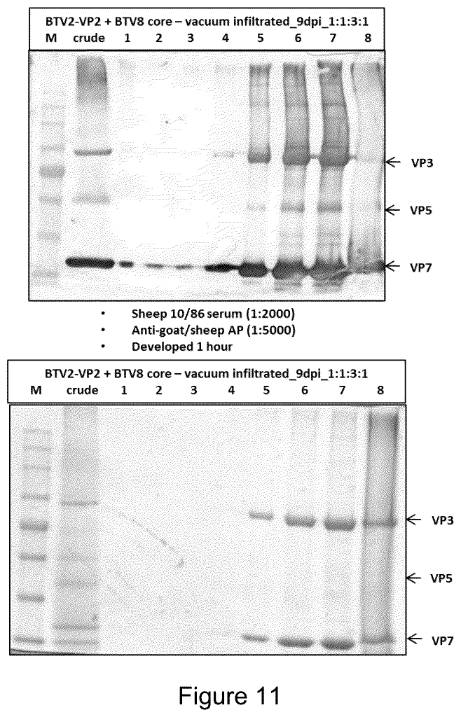

In previous work on BTV-8 VLPs only it was shown that an infiltration ratio of 1:1:2:1 of VP2:VP3:VP5:VP7 yielded the best VLPs and this was tested in this chimaeric constructs to try and skew production from more CLPs to more VLPs. FIG. 11 shows the western blot and cognate Coomassie-stained gel of fractions purified on the same gradient as above. VP3, VP5 and VP7 are clearly visible in fractions 5 to 7 on the western blot and in the Coomassie-stained gel and VP3, VP5 and VP7 are visible although there is much less VP5 than VP3 or VP7.

This example proves that it is possible to make chimaeric BTV VLPs although the infiltration process needs to be optimised in order to direct the preferential assembly of VLPs rather than CLPs.

Although BTV-8 VP3, VP5 and VP7 proteins are detectable by western blot and BTV-2 VP2 proteins are not, chimaeric BTV VLPs comprising of BTV-8 VP3, VP5 and VP7 and BTV-2 VP2 can be produced in plants although it seems there are significantly more core-like particles (CLPs) and sub-core-like particles (sCLPs) made than VLPs.

### Example 3

The following plant codon optimised nucleotide sequences were synthesised by Bio Basic Int.™: BTV-3 VP2 (SEQ ID NO:29), BTV-3 VP5 (SEQ ID NO:32), BTV-4 VP2 (SEQ ID NO:30), BTV-4 VP5 (SEQ ID NO:33), BTV-4 VP7 (SEQ ID NO:34), BTV-8 VP2 (SEQ ID NO:31), BTV-8 VP3 (SEQ ID NO:22), BTV-8 VP5 (SEQ ID NO:24) and BTV-8 VP7 (SEQ ID NO:26). The plant codon optimised nucleotide sequences encode the following proteins: BTV-3 VP2 (SEQ ID NO:9), BTV-3 VP5 (SEQ ID NO:12), BTV-4 VP2 (SEQ ID NO:10), BTV-4 VP5 (SEQ ID NO:13), BTV-4 VP7 (SEQ ID NO:14), BTV-8 VP2 (SEQ ID NO:11), BTV-8 VP3 (SEQ ID NO:2), BTV-8 VP5 (SEQ ID NO:4) and BTV-8 VP7 (SEQ ID NO:6).

Using the protocols described herein the nucleotide sequences where cloned into the pEAQ-HT expression vector and the vectors were subsequently electroporated into Agrobacterium tumefaciens LBA4404 (1.44 kV, 200 (and 25 μF). Similarly PEAQ-HT void of an insert were also electroporated into Agrobacterium and served as negative controls. A PEAQ-HT-gfp vector containing the green fluorescent protein gene (gfp) served as positive control. The product was resuspended in Luria broth medium, and placed on a rotor shaker at 28° C. for three hours to recover before plated on selective medium (50 mg/l streptomycin, 25 mg/l kanamycin and 20 mg/l Rifamycin). A single colony of each insert was PCR validated using PEAQ-HT Fw (SEQ ID NO:49) and pEAQ-HT Rv (SEQ ID NO:50) primers. These primers are specific for the pEAQ-HT vector.

TABLE 2
pEAQ-HT plasmid specific primers.

| Primer Name | Sequence | SEQ ID NO. |
|---|---|---|
| pEAQ-HT Fw | 5′ ACTTGTTACGATTCTGCTG | (SEQ ID NO: 49) |
| | ACTTTCGGCGG 3′ | |
| pEAQ-HT Rv | 5′ CGACCTGCTAAACAGGAGC | (SEQ ID NO: 50) |
| | TCACAAAGA 3′ | |

Transient expression efficiency of the pEAQ series of vectors was investigated by agroinfiltration of Nicotiana benthamiana. The assembly of VLPs was also validated in N. benthamiana which facilitates mammalian-like or human-like glycosylation RNAi mutant N. benthamiana dXT/FT (Strasser et al., 2008). The pEAQ-HT constructs containing genes encoding individual capsid proteins of BTV serotypes 3, 4 and 8 were individually transformed into Agrobacterium. Prior to plant infiltration Agrobacterium tumefaciens strain (LBA4404) transformed with PEAQ-HT vector containing individual VP2, VP3, VP5 and VP7 of selected serotypes were streaked on YMB agar plates and incubated at 28° C. for 48 hrs. The growing bacterium was scraped off from the plate and inoculated into YMB broth with the relevant antibiotics and grown overnight. Cells were pelleted and resuspended in MMA buffer (100 mM MES, 10 mM MgCl₂ and 100 mM acetosyringone; pH 5.6). Each of the four Agrobacterium cultures were adjusted to OD₆₀₀ of approximately 0.5-0.7 with the same buffer. The formation of VLPs was validated by mixing and infiltrating the four constructs encoding the four individual capsid proteins at a ratio of 1:1:1:1 and used for plant infiltrations (20-25 plants; 15-20cm in height). The Agrobacterium transformed with pEAQ-HT-gfp was used as the positive control and negative was A. tumefecians transformed with an empty pEAQ-HT vector. The construct pEAQ-HT-VP3 was sometimes substituted with PEAQ-VP3wt, to suppress the expression of capsid protein VP3 and to shift the stoichiometry from core-like particles (CLPs) to VLPs as previously described (Theunemann, et al., 2013).

The leaf material was harvested four to eight days after infiltration using a Matstone Multipurpose™ juice extractor in VLP extraction buffer (50 mM bicine, pH 8.4; 20 mM sodium chloride [NaCl], 0.1% (w/v) N-lauroylsarcosine (NLS) sodium salt; 1 mM dithiothreitol (DTT)) in a ratio 1:3 with complete protease inhibitor cocktail (P2714, Sigma Life Sciences™) or complete EDTA-free tablets (Roche™) added to the VLP extraction buffer immediately before the extraction started. In a particular experiment, 0.5 mM CaCl₂ was added to the extraction medium to potentially stabilize the formed VLPs. Crude extracts were centrifuged twice for 10 minutes at 4,200×g, 10° C. to remove cell debris in a JA14 rotor using a Beckman Coulter™ Avanti J-26 XPI centrifuge.

Particles were purified by density gradient centrifugation using ultra-high quality sucrose (Sigma Life Sciences™) step gradients (30%-70%) prepared dissolved in VLP dilution buffer (50 mM Tris-HCl, pH 8.4, 20 mM NaCl). Step gradients of 1 ml with 10% incrementing steps were prepared and then overlaid with 8 ml of clarified leaf extract. The gradients were centrifuged at 85,800×g, at 10° C. for 3 hours in a SW-41Ti rotor (Beckman Coulter™). Fractions of 500 μl were collected and aliquots (26 μl) from all fractions were analysed on 4-12% Bis-Tris Bolt™ (Life Technologies™) protein gels or 10% Stain Free SDS-PAGE (Bio-Rad™). The sucrose-gradient purified product was dialysed overnight against bicine buffer containing only the bicine (pH 8.4) and sodium chloride in preparation for animal trials.

The sucrose gradient fractions were adsorbed onto holey carbon-coated copper grids as follows. The grids were floated on the 1/10 dilution protein sample for 30 seconds and excess sample drained off the grid via blotting on filter paper. Subsequently the grid was floated on 2% sodium phophotungstate, pH 7.0 for 30 seconds (0.22 μm filter sterilized before staining) and drained as described above. The air dried grid was imaged in a JEM-2100 Transmission electron microscope (JEOL™) at the University of Pretoria, Laboratory for Microscopy and Microanalysis.

Protein bands of interest were in-gel trypsin digested as per the protocol described in (Shevchenko et al., 2007). In short, gel bands were destained using 50 mM NH₄HCO₃/50% MeOH followed by in-gel protein reduction (50 mM DTT in 25 mM NH₄HCO₃) and alkylation (55 mM iodoacetamide in 25 mM NH₄HCO₃). Proteins were digested over night at 37° C. using 5-50 μl, 10 ng/μl trypsin depending on the gel piece size. Digests were resuspended in 20 μl, 2% acetonitrile/0.2% formic acid and analysed using a Dionex Ultimate 3000 RSLC™ system coupled to an AB Sciex 6600 TipleTOFT mass spectrometer. Peptides were first de-salted on an Acclaim PepMap C18™ trap column (100 μm×2 cm) for 2 min at 15 μl/min using 2% acetonitrile/0.2% formic acid, than separated on Acclain PepMap C18 RSLC™ column (300 μm×15 cm, 2 μm particle size). Peptide elution was achieved using a flow-rate of 8 μl/min with a gradient: 4-60% B in 15 min (A: 0.1% formic acid; B: 80% acetonitrile per 0.1% formic acid). An electrospray voltage of 5.5 kV was applied to the emitter. The 6600 TipleTOF™ mass spectrometer was operated in Data Dependant Acquisition mode. Precursor scans were acquired from m/z 400-1500 using an accumulation time of 250 ms followed by 30 product scans, acquired from m/z 100-1800 at 100 ms each, for a total scan time of 3.3 sec. Multiply charge ions (2+-5+, 400-1500 m/z) were automatically fragmented in Q2 collision cells using nitrogen as the collision gas. Collision energies were chosen automatically as function of m/z and charge.

Protein pilot v5 using Paragon™ search engine (AB Sciex™) was used for comparison of the obtained MS/MS spectra with Uniprot Swissprot protein database. Proteins with threshold above ≥99.9% confidence were reported.

Assembly of BTV serotype 3, 4 and 8 VLPs were investigated by infiltrating N. benthamiana mammalian-like mutant dXT/FT or unmodified N. benthamiana plants with the relevant constructs. The Agrobacterium strain LBA4404 harboring PEAQ-HT constructs encoding for the four capsid proteins individually for BTV serotypes 3, 4 and 8 were successfully infiltrated into N. benthamiana leaves. Production of VLPs in plant leaf tissue was determined by mixing the four constructs encoding the four individual capsid proteins VP2:VP3:VP5:VP7 at a ratio of 1:1:1:1. Leaf tissue was harvested eight days after infiltration, extracted and purified as described.

The production of all four capsid proteins was determined by SDS-PAGE and immuno blot analysis where appropriate serum was available. The crude leaf extract was subjected to sucrose gradient purification and distinct protein bands were identified on Coommassie stained SDS-PAGE gels. The protein bands at 111 kDa, 100 kDa, 59 kDa and 38 kDa were confirmed to be the capsid proteins VP2, VP3, VP5 and VP7, respectively, by mass spectrophotometry. The assembly of VLPs (˜70 nm) was confirmed by transmission electron microscopy (TEM) for all three serotypes 3, 4 and 8.

The following combinations of capsid proteins, extraction buffer composition and protease inhibitors were tested for assembling and purification of chimaeric CLPs and chimaeric VLPs.

### BTV-3 Double Chimaeric

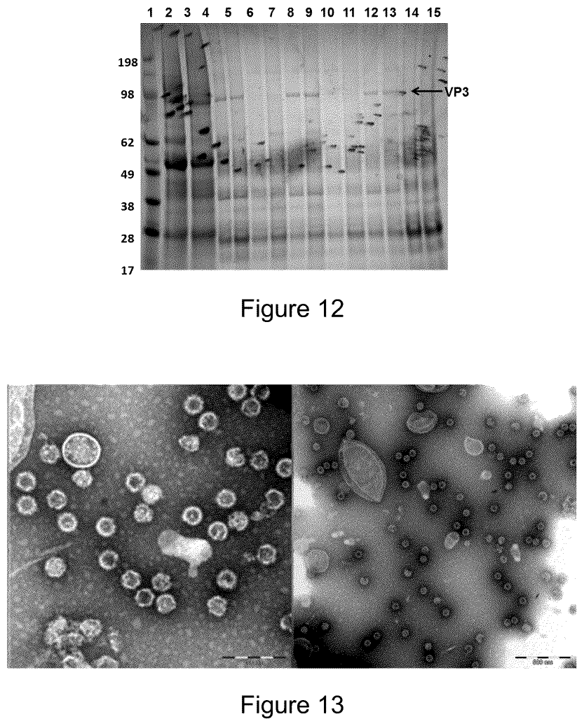

Genes encoding BTV-8 VP3 and BTV-8 VP7 forming CLPs combined with BTV-8 VP5 and BTV-3 VP2 (BTV-3 single chimaeric) or BTV-8 VP3, BTV-8 VP7 combined with BTV-3 VP2, BTV-3 VP5 (BTV-3 double chimaeric) for the outer capsids were combined as described and infiltrated into N. benthamiana leaves. Alternatively, BTV-8 VP3 cloned into PEAQ wild type (wt) was used instead of BTV-8 VP3 cloned into pEAQ-HT to form the core and in an attempt to improve the stoichiometry towards VLPs as described before (Theunemann et al., 2013). Although VP3 is clearly reduced when using BTV-8 VP3wt in forming the cores (FIG. 12, lanes 6-7, 10-11 and 14-15), the VLP vs CLP stoichiometry did not improve in our hands.

VLPs were extracted in standard bicine buffer containing protease inhibitor (Sigma™ P2714-1BTL) and purified using a sucrose gradient (30-70%) and fractions (45-55%) were analyzed using Bolt™ gels. The presence of the core capsid proteins VP3 and VP7 as well as one of the outer capsid proteins VP5 were consistently detected at ˜100 kDa, 38 kDa and 59 kDa, respectively and therefore not repeatedly subjected to mass spectrometry. The detection of VP2 with mass spectrometry and TEM analysis were considered sufficient to confirm the formation of VLPs for all future experiments.

Creation of Chimaeric VLPs Comprising BTV-8 VP3 and BTV-4 VP2, VP5 and VP7 in Mammalian-Like Tobacco dXT/FT

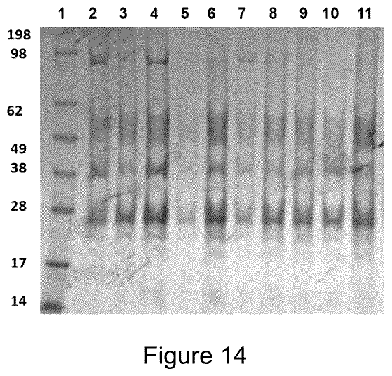

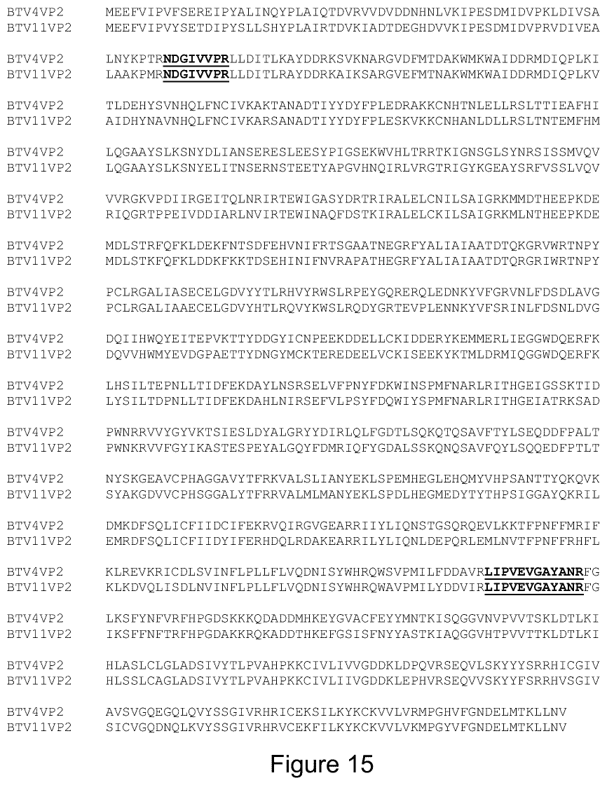

Genes encoding BTV-8 VP3 and BTV-8 VP7 were used to assemble and form CLPs in mutant N. benthamiana dXT/FT tobacco (FIG. 13). BTV-4 VP2, VP5 and VP7 were used in combination with BTV-8 VP3 to form VLPs i.e. only BTV-8 VP3 core capsid with all the remaining capsids from BTV-4 (VP2, VP5 and VP7). CLPs and VLPs were extracted in bicine buffer containing protease inhibitor (Sigma™ P2714-1BTL) and purified using a sucrose gradient (30-70%). Selected fractions were analyzed using Bolt™ gels as described (FIG. 14). The presence of BTV-4 VP2 was confirmed by mass spectrometry. Although the peptides detected were indicated as BTV-11, identical sequences appear in BTV-4 (FIG. 15).

### Creation of Double Chimaeric BTV-3 VLPs

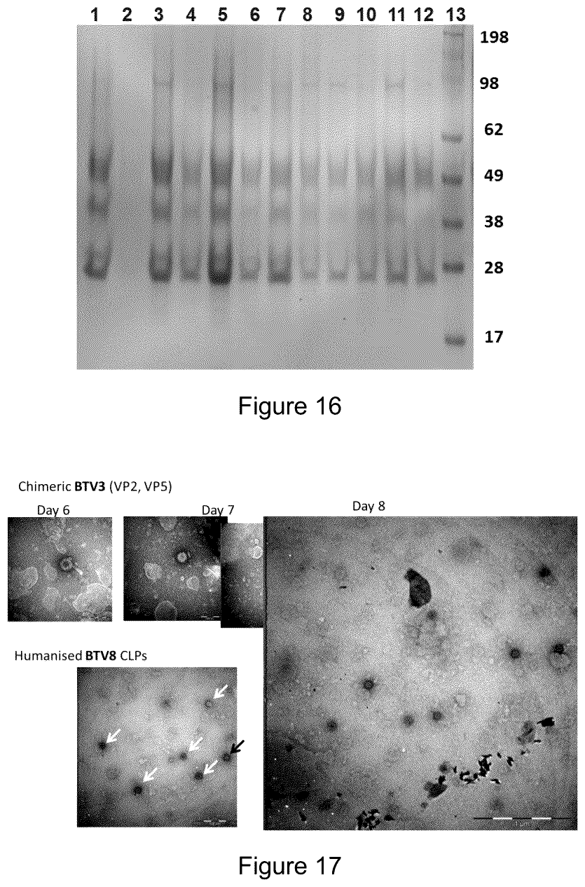

BTV-8 VP3 and VP7 inner capsid proteins were assembled with BTV-3 VP2 and VP5 outer capsid proteins. VLPs were extracted in bicine buffer containing protease inhibitor (Sigma™ P2714-1BTL) and purified using a sucrose gradient (30-70%) and fractions (45-40%) were analyzed using Bolt™ gels as described (FIG. 16). BTV-3 VP2 peptides were detected 6-8 days after infiltration of N. benthamiana and the mutant N. benthamiana dXT/FT. BTV-8 CLPs and BTV-3 VLPs were also visualized under the TEM (FIG. 17). As VLPs were detected more abundantly eight days after infiltration, the leaf material was harvested 8 days after infiltration and subjected to sucrose gradient purification for all future experiments.

### Chimaeric BTV-4 and BTV-3 VLPs in Humanized Tobacco

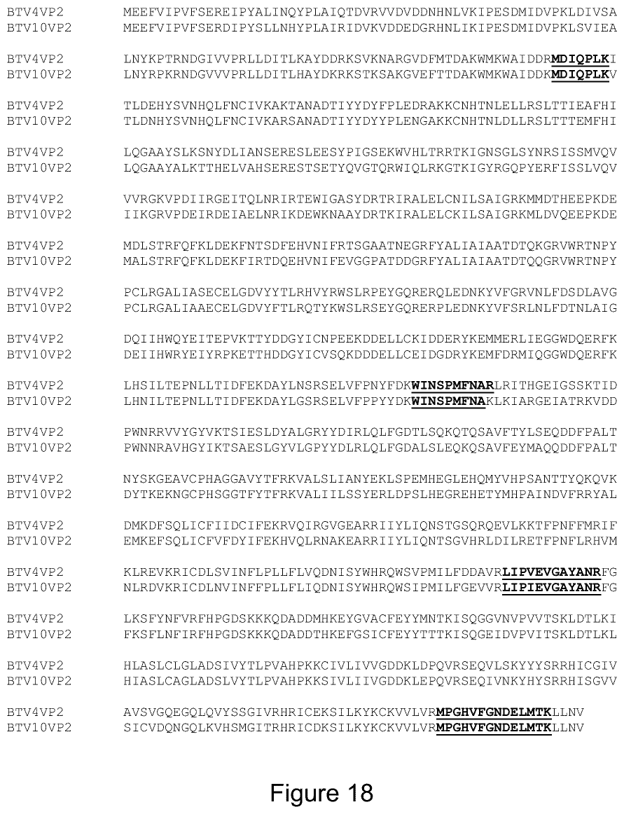

Previously, BTV VLPs were assembled with BTV-8 VP3 and BTV-4 VP2, VP5 and VP7. In this experiment double chimaeric BTV VLPs were assembled with BTV-8 VP3 and VP7 cores and BTV-4 VP5 and VP2 outer capsids, alternatively double chimaeric BTV VLPs were assembled with BTV-8 VP3 and VP7 cores and BTV-3 VP5 and VP2 outer capsids. Double chimaerics with BTV-8 VP3 and VP7 forming the core and the outer capsid proteins being from a second serotype (3 or 4) seem to be more stable than single chimaeric BTV VLPs. The double chimaeric VLPs will be used for sheep trials. The BTV VLPs were extracted in bicine buffer supplemented with Roche™ EDTA-free protease inhibitor. Mass spectrometry confirmed the presence of BTV-3 VP2 and BTV-4 VP2. Although the peptides detected were indicated as BTV-10, the inventors point out that identical sequences appear in BTV-4 (FIG. 18).

### Purifying Double Chimaeric BTV VLPs in Buffer Containing CaCl₂ and With Different Protease Inhibitors

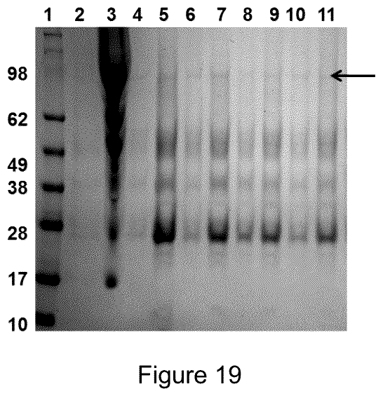

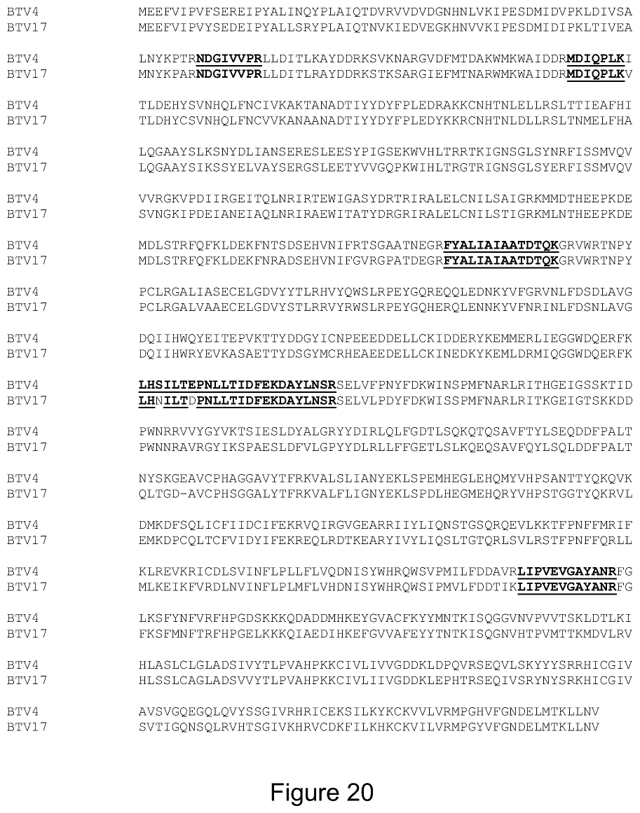

Two independent protease inhibitors and the addition or omission of CaCl₂ were compared to identify which method best preserved the formed VLPs during extraction. Chimaeric BTV VLPs were assembled with BTV-8 VP3 and VP7 cores and BTV-4 VP5 and VP2 outer capsids. The expression and assembly of capsid proteins was conducted in N. benthamiana dXT/FT. Selected sucrose gradient fractions were separated using the Bolt™ 4-12% SDS PAGE gels and fragments at ˜100-120 kDa were subjected to mass spectrometry (FIG. 19). BTV-4 VP2 was detected when the BTV-4 VLPs were extracted in bicine buffer using either Sigma™ protease inhibitor or the Roche™ EDTA-free protease inhibitor. The VP2 peptides (five in total) detected with mass spectrometry, were identified as BTV-17 due to a difference of only two amino acids (FIG. 20). The addition of CaCl₂ did not enhance the amount of VLPs purified.

### Example 4

Plant expressed AHS single, double and triple chimaeric VLPs Gene sequences, encoding the VP2, VP5, VP3 and VP7 proteins of AHSV serotype 1 (Genbank™ accession numbers AM883165, FJ183369, FJ183366, AM883171, respectively), the VP2 (Genbank™ accession number AY163330) and VP5 (Genbank™ accession number JQ742011) proteins of AHSV serotype 7, the VP5 protein of AHSV serotype 3 (Genbank™ accession number DQ868777and the VP2 protein of AHSV serotype 6 (Genbank™ accession number DQ868774.1) were codon optimised for optimal expression in Nicotiana benthamiana plant cells and synthesized with unique AgeI and XhoI sites at the 5′ and 3′ termini, respectively.

The following plant codon optimised nucleotide sequences were synthesised by BioBasic™ Inc, Canada: AHSV-1 VP2 (SEQ ID NO:38), AHSV-1 VP3 (SEQ ID NO:35), AHSV-1 VP5 (SEQ ID NO:36), AHSV-1 VP7 (SEQ ID NO:37), AHSV-7 VP2 (SEQ ID NO:39), AHSV-7 VP5 (SEQ ID NO:40), AHSV-3 VP5 (SEQ ID NO:65) and AHSV-6 VP2 (SEQ ID NO:67). These plant codon optimised nucleotide sequences encode the following proteins: AHSV-1 VP2 (SEQ ID NO: 18), AHSV-1 VP3 (SEQ ID NO:15), AHSV-1 VP5 (SEQ ID NO:16), AHSV-1 VP7 (SEQ ID NO:17), AHSV-7 VP2(SEQ ID NO:19), AHSV-7 VP5 (SEQ ID NO:20), AHSV-3 VP5 (SEQ ID NO:66) and AHSV-6 VP2 (SEQ ID NO:68).

The VP2, VP5, VP3 and VP7 nucleotide sequences were subsequently cloned into the pEAQ expression vectors (Sainsbury et al., 2009, Plant Bioscience Limited, UK). More, specifically sequences encoding the AHSV-1 VP5, VP3 and VP7 proteins were firstly cloned into the intermediate PEAQ vectors FSC5 or FSC6 via directional AgeI/XhoI restriction enzyme-based cloning. The restriction enzymes in this study were supplied by ThermoScientific™ and the Fast-link™ DNA ligase enzyme by EpiCentre™. Cloning of the AHSV-1 VP2-encoding sequence into the intermediate FSC5 vector was performed using the In-Fusion HDR cloning kit (Clontech™) with the primers depicted in Table 3, according to the manufacturers instructions.

TABLE 3
In-Fusion AHSV specific primers.

| Primer | | SEQ ID |
|---|---|---|
| Name | Sequence | NO. |
| In-Fusion | 5′ CAAATTCGCGACCGGTCCAT | (SEQ ID |
| AHSVP2-F | GGCTAGTGAATTC 3′ | NO: 51) |
| In-Fusion | 5′ AGTTAAAGGCCTCGAGTTAT | (SEQ ID |
| AHSVP2-R | TCTATCTTTGAAAGC 3′ | NO: 52) |
| In-Fusion | 5′ CAAATTCGCGACCGGTCCAT | (SEQ ID |
| HS5VP2-F | GGTTCAGAATTCGGTG 3′ | NO: 69) |
| In-Fusion | 5′ AGTTAAAGGCCTCGAGTCAT | (SEQ ID |
| HS5VP2-R | TTCTCGGTTTTGGCC 3′ | NO: 70) |
| In-Fusion | 5′ CAAATTCGCGACCGGTCCAT | (SEQ ID |
| HS6VP2-F | GGCTTCTGAATTCGGT 3′ | NO: 71) |
| In-Fusion | 5′ AGTTAAAGGCCTCGAGTCAC | (SEQ ID |
| HS6VP2-R | TCGGCTTTGGCCAT 3′ | NO: 72) |

The VP5-encoding expression cassette was subsequently cloned from the recombinant FSC6-VP5 plasmid into the PEAQ express vector via directional AscI/SbfI restriction enzyme-based cloning. Cloning of the VP7-encoding expression cassette from FSC6-VP7 into the pEAQ express vector followed a similar process except that the recombinant plasmid was digested with both enzymes AscI and AlwI prior to digestion with SbfI to ensure different sizes of insert and vector backbone DNA fragments. Cloning of the VP2 and VP3 encoding sequences into the linearized pEAQ-HT vector required that the respective FSC5-VP2 and FSC-5-VP3 recombinant plasmids be digested with the AgeI and XhoI prior to ligation. The AHSV-7 VP2 and AHSV-7 VP5 encoding sequences were cloned individually into the pEAQ-HT vector via directional AgeI/XhoI restriction enzyme-based cloning. Cloning of the sequences encoding the AHSV-3 VP5 and AHSV-6 VP2 proteins individually into the pEAQ-HT vector was performed using the In-Fusion HDR cloning kit (Clontech) with the primers depicted in Table 3, according to the manufacturers instructions.

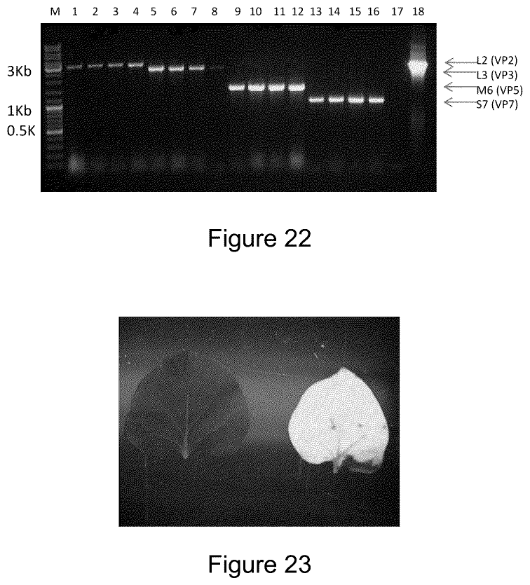

In order to generate the dual recombinant plasmid PEAQ-express-AHSV-1VP3-AHSV1-VP7, the AHSV-1 VP7 encoding sequence was firstly cloned from PEAQ-express-AHSV-1VP7 plasmid into the pEAQ-HT vector via directional AgeI/XhoI restriction enzyme-based cloning. The VP7-encoding expression cassette was subsequently excised from pEAQ-HT-AHSV-1VP7 using the AscI/PacI enzymes and cloned into the compatible MluI/AsiSI sites of the pEAQ-express vector. The VP3-encoding expression cassette was transferred from the pEAQ-HT-AHSV1VP3 plasmid into the newly generated pEAQ-express-AHSV1VP7 plasmid via AscI/PacI mediated restriction enzyme based cloning. Following transformation into electrocompetant DH10B bacterial cells, the presence of recombinant plasmid in candidate bacterial clones was verified via colony PCR with the primers depicted in Table 4. The presence of the AHSV-1 L2 (VP2), L3 (VP3), M6 (VP5) and S7 (VP7) PCR products can be visualised in FIG. 22 following agarose gel electrophoresis.

TABLE 4
Primers used for colony PCR.

| Pro- | Primer | | SEQ ID |
|---|---|---|---|
| tein | Name | Sequence | NO. |
| AHSV-1 | QAHSVP2- | 5′ CGTACCGGTCCATGGCT | (SEQ ID |
| VP2 | F | AGTGAATTCGGT 3′ | NO: 53) |
| | QAHSVP2- | 5′ GCAGCTCGAGTTATTCT | (SEQ ID |
| | R | ATCTTTGAAAGC 3′ | NO: 54) |
| AHSV-1 | QAHSVP3- | 5′ GGTACCGGTATGCAAGG | (SEQ ID |
| VP3 | F | TAACGAACGT 3′ | NO: 55) |
| | QAHSVP3- | 5′ CAGCTCGAGTTAAATTG | (SEQ ID |
| | R | TTGGCCTTGC 3′ | NO: 56) |
| AHSV-1 | QAHSVP5- | 5′ CGTACCGGTCCATGGGA | (SEQ ID |
| VP5 | F | AAATTTACTTC 3′ | NO: 57) |
| | QAHSVP5- | 5′ CAGCTCGAGTTAGCTAA | (SEQ ID |
| | R | TCTTCACGCC 3′ | NO: 58) |
| AHSV-1 | QAHSVP7- | 5′ GCTACCGGTCCATGGAT | (SEQ ID |
| VP7 | F | GCAATAGCAGC 3′ | NO: 59) |
| | QAHSVP7- | 5′ CAGCTCGAGTTAATGAT | (SEQ ID |
| | R | AAGCTGCAAG 3′ | NO: 60) |
| AHSV-7 | AHSV7VP2F | 5′ CCATGGCATCAGAGTTT | (SEQ ID |
| VP2 | | GGTATC 3′ | NO: 61) |
| | AHSV7VP2R | 5′ CCTCATTCTGCCTTTGA | (SEQ ID |
| | | TAACAGC 3′ | NO: 62) |
| AHSV-7 | AHSV7VP5- | 5′ ACCGGTATGGGAAAGTT | (SEQ ID |
| VP5 | F | C 3′ | NO: 63) |
| | AHSV7VP5- | 5′ CTCGAGGGCAATACGAA | (SEQ ID |
| | R | C 3′ | NO: 64) |
| AHSV-5 | FSC5F | 5′ GGTTTTCGAACTTGGAG | (SEQ ID |
| VP2 | | AAA 3′ | NO: 73) |
| | FSC5R | 5′ AGAAAACCGCTCACCAA | (SEQ ID |
| | | ACATAGA 3′ | NO: 74) |
| AHSV-6 | FSC5F | 5′ GGTTTTCGAACTTGGAG | (SEQ ID |
| VP2 | | AAA 3′ | NO: 75) |
| | FSC5R | 5′ AGAAAACCGCTCACCAA | (SEQ ID |
| | | ACATAGA 3′ | NO: 76) |

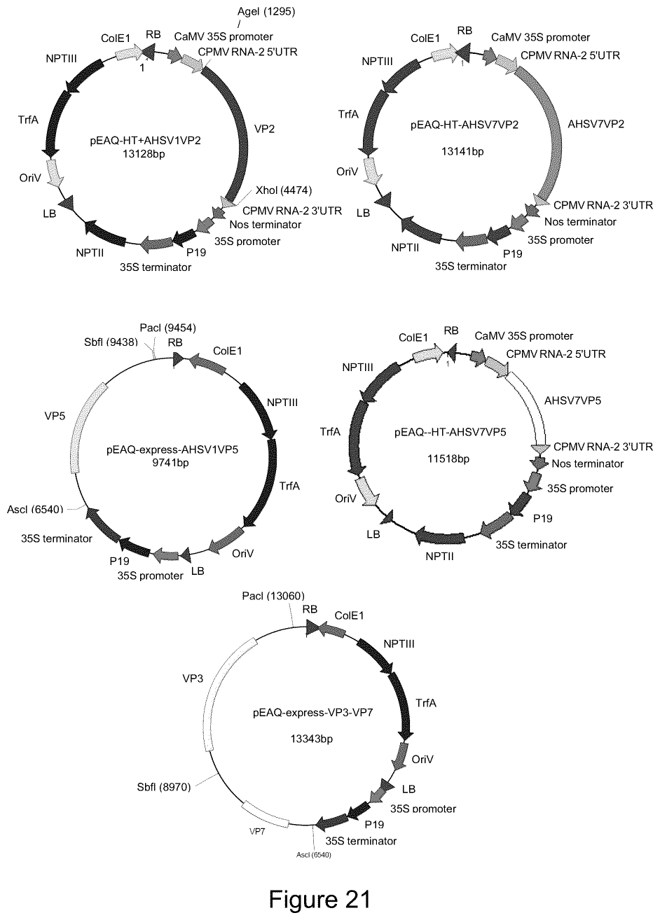

The PCR reactions contained a final concentration of 0.3 M forward/reverse primer and the KAPA 2G Fast DNA polymerase enzyme (KAPA Biosystems™) and were set up according to the manufacturer's instructions. The cycling conditions were as follows: 1 cycle of 95° C. for 2 min, followed by 25 cycles of 95° C. for 20 sec, 59° C. (47° C. for AHSV-7 VP2 and AHSV-7 VP5) for 15 sec and 72° C. for 3 min 30 sec followed by 1 cycle of 72° C. for 7 min. The capsid protein encoding sequences were verified via dideoxy Sanger DNA sequencing (Inqaba Biotechnical Industries (Pty) Ltd™). Some of the recombinant plasmids constructed are depicted in FIG. 21.

Transient expression of the AHSV capsid proteins was accomplished via Agrobacterium-mediated infiltration of Nicotiana benthamiana or Nicotiana benthamiana dXT-FT plant leaves with the recombinant PEAQ expression plasmids. One hundred nanograms of the recombinant pEAQ plasmid was transformed into 60 μl electrocompetant LBA4404 Agrobacterium cells (1.44 kV, 200Ω and 25 μF) using a Gene Pulsar™ (Bio-Rad™). The transformed bacterial cells were resuspended in 500 μl SOC medium and placed on a rotational shaker (175 rpm) at 30° C. for 3 hours to recover prior to 250 μl being plated out onto two selective medium plates (50 μg/ml Kanamycin, 50 μg/ml Rifampicin and 50 μg/ml Streptomycin). The plates were inverted and incubated at 28° C. for 96 hours. All reagents were molecular biology grade and obtained from Sigma® Life Science unless otherwise indicated. Recombinant LBA4404 bacterial clones, verified via colony PCR, were inoculated into 5 ml YMB medium (0.1% yeast extract, 1% Mannitol, 1.7 mM NaCl, 0.8 mM MgSO₄·H₂O, 2.2 mM K₂HPO₄), with the appropriate antibiotics (50 μg/ml Kanamycin, 50 μg/ml Rifampicin and 50 μg/ml Streptomycin), and incubated with rotational shaking (175 rpm) for 24 hours at 28° C. Cryopreserved LBA4404 Agrobacterium cells, containing the pEAQ-HT vector or the pEAQ-HT-GFP plasmid, were also inoculated into 5 ml YMB media to serve as negative and positive controls, respectively. The Agrobacteria starter cultures were subsequently used to inoculate 50 ml YMB media with the appropriate antibiotics and these cultures were incubated overnight at 28° C. with rotational shaking (175 rpm). The bacterial cells were harvested from the overnight cultures via centrifugation at 8000 rpm for 7 min at 20° C. The cell pellets were each resuspended in 40 ml freshly prepared MMA infiltration buffer (10 mM MES hydrate; pH 5.6, 10 mM MgCl₂, 100 μM 3,5-dimethoxy-4-hydroxy-acetophenone). In order to assess the assembly of CLPs, N. benthamiana leaves were agroinfiltrated with the VP7 and VP3-encoding genes, whilst agroinfiltration with all four capsid protein encoding sequences enabled assessment of VLP assembly. The agrobacterial suspensions were combined in a 1:1:1 ratio (for VLPs) and 1:1 (for CLPs) and subsequently diluted with the MMA buffer such that the final OD₆₀₀ was 0.45-0.5. The leaves of four week old N. benthamiana plants were syringe-infiltrated with these Agrobacteria combinations or the pEAQ-HT/PEAQ-HT-GFP Agrobacteria suspension. The plants were incubated at 27° C. for 8 days post-infiltration (dpi).

Preliminary evidence of foreign protein expression was obtained by illuminating the pEAQ-HT-gfp-infiltrated leaf with UV light 8 dpi (FIG. 23). The visualization of fluorescing fluorescent green fluorescent protein (gfp) protein within the infiltrated leaf indicated that the infiltration procedure had been successful and it was thus likely that the AHSV capsid proteins had also been expressed in their respective infiltrated N. benthamiana leaves.

Agrobacterium infiltrated N. benthamiana leaves were photographed and harvested 8 days post-infiltration. The leaf tissue was extracted immediately in 3 volumes of VLP extraction buffer (20 mM sodium chloride (NaCl), 50 mM Bicine, pH 8.4, 0.1% (w/v) sodium lauroyl sarcosine (NLS), 1 mM dithiothreitol (DTT) (ThermoScientific™), 0.2% protease inhibitor cocktail P2714 (Sigma® Life Science)/complete EDTA-free protease inhibitor cocktail (Sigma-Aldrich®) or CLP extraction buffer (as VLP extraction buffer but containing 140 mM NaCl) in a multipurpouse juice extractor (MATSONE™). The DTT and protease inhibitor cocktails were freshly prepared according to the manufacturers' instructions and added to the extraction buffer just prior to use. Large cell debris was removed by filtering the cell lysate through two layers of Miracloth™ and the extract further clarified via centrifugation (4200×g; 30 min; 10° C.).

Virus-like particles (VLPs) or Core-like particles (CLPs) were purified using sucrose density gradient centrifugation. Sucrose solutions (30%-70%) were prepared by dissolving ultra-high quality sucrose (Sigma® Life Science) in VLP dilution buffer (20 mM NaCl, 50 mM bicine, pH 8.4) or CLP dilution buffer (140 mM NaCl, 50 mM Bicine, pH 8.4) and layered into gradients of 1 ml 10% incrementing steps. The clarified cell lysates were layered on top of the sucrose gradients and centrifuged in a SW-41Ti rotor (Beckman Coulter™) at 85,800×g for 3 hours; 10° C. The 55%-35% sucrose layers were harvested in 500 μl fractions using a Minipuls2 peristaltic pump (Gilson™). Ten microlitres of each fraction was subsequently analysed for protein content by denaturing SDS-PAGE and immunoblotting procedures.

Ten microliter of each sucrose fraction was mixed with an equal volume of 2× Laemmli protein sample buffer (4% SDS, 20% glycerol, 10% 2-mercaptoethanol, 0.004% bromophenol blue and 0.125 M Tris HCl, pH approx. 6.8), the protein samples denatured at 95° C. for 5 min and analysed on denaturing SDS 10% polyacrylamide gels (BioRad TGX Stain Free™ Fast Cast™), prepared according to the manufacturer's instructions. The Precision Plus Protein™ WesternC™ standard (Bio-Rad™) was used as a size marker. Electrophoresis was performed in 1×TGS buffer (25 mM Tris-HCl; pH 8.3, 200 mM glycine, 0.1% SDS) using the Mini-PROTEAN® Tetra system (Bio-Rad™) by applying a current of 50 V for 20 min and thereafter a current of 130 V for approximately 1.5 hours. The polyacrylamide gels were then subjected to an immunoblot protocol with an AHSV-7 specific polyclonal antiserum to confirm the identity and position of the AHSV capsid proteins on the gels.

The protein samples were immunoblotted onto a PVDF membrane within the Trans-blot® Turbo™ Transfer Pack (Bio-Rad™) using the Trans-Blot®Turbo™ Transfer system (Bio-Rad™) mixed MW application (1.3 A; 25 V; 7 min). The membrane was incubated in 3% blocking solution (3% bovine serum albumin (Roche™) in 1×Tris buffered saline (150 mM NaCl, 20 mM Tris pH 7.5; 0.1% Tween®-20 (Merck™)) at room temperature with gentle agitation for 3 hours. Prior to incubation with the membrane, the primary antibody, an anti-AHSV-7 guinea pig polyclonal antiserum (GPaAHSV-7), was pre-treated with N. benthamiana plant extract in order to remove plant protein-specific antibodies from the serum. This was done by crushing a single uninfiltrated N. benthamiana leaf in a mortar and pestle with 1×TBS buffer in a ratio of 1:3, adding the primary antibody to this leaf extract and incubating this mixture at 37° C. for 1 hour with slight agitation. This plant extract/primary antibody mixture was then added to 3% blocking solution (1:300 dilution) and incubated overnight at 4° C. with gentle agitation to allow binding of the antibodies to immobilised protein.

The membrane was subsequently washed five times with wash buffer (0.1% Tween® 20 (Merck™) in 1×TBS), 5 min for each wash. A secondary antibody, a combination of the horseradish peroxidase-conjugated Rabbit anti-Guinea Pig IgG H&L (HRP) conjugate (abcam® ab6771) (1:5000 dilution) and Precision Protein™ StrepTactin-HRP Conjugate (Bio-Rad™) (1:10000 dilution) was then added. After incubation at room temperature for 1 hour with gentle agitation, the membrane was washed five times in wash buffer (0.1% Tween® 20 (Merck™) in 1×TBS), 5 min each wash. The membrane was then subjected to the detection procedure by adding the Clarity Western™ ECL chemilluminescent substrate (Bio-Rad™), according to the manufacturer's instruction, and placing the membrane immediately into the ChemiDoc™ MP Imager (Bio-Rad). By using the Chemi Hi Resolution™ application, photographs of the chemilluminescence signals were taken approximately every second with the accumulating exposure starting at 1 second and ending at 15 seconds.

Sucrose fractions containing putative AHSV capsid proteins were electrophorized on precast denaturing 4-12% Bolt™ Bis-Tris Plus polyacrylamide Gels (Thermo Fischer Scientific™), according to the manufacturers' instructions. SeeBlue® Plus2 Prestained Protein Standard (Invitrogen™) was used as the size marker. Electrophoresis was performed in the Bolt® MES or MOPS SDS running buffer using the mini gel tank (Thermo Fischer Scientific™) by applying a current of 200 V for approximately 35 min. The gels were then stained in Coomassie Brilliant Blue G250 staining solution (50% methanol (Minema™), 10% acetic acid (Minema™), 0.1% Coomassie Brilliant Blue G250 (Merck™)) for 20 min and destained in destaining solution (10% methanol, 10% acetic acid) overnight. Candidate protein bands of approximately the correct size were excised from the gel and sent for Mass spectrometry (MS) analysis (Dr Stoyan Stoychev, CSIR Biosciences).

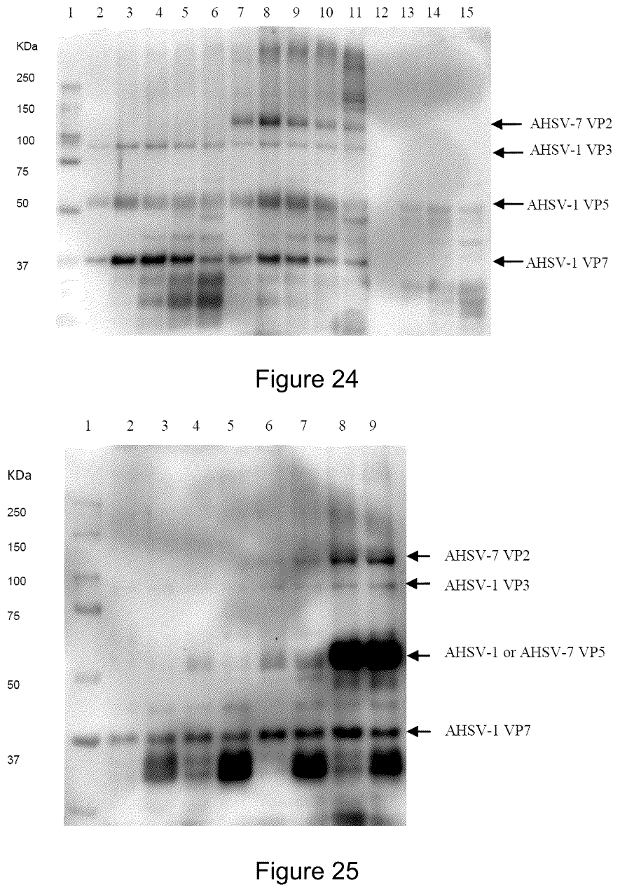

Expression of the AHSV-1 VP3, VP7, VP5 and AHSV-7 VP2 capsid proteins in the Nicotiana benthamiana leaves, harvested at 8 dpi, was confirmed via sucrose density centrifugation and immunoblot analysis with guinea pig αAHSV-7 serum (FIG. 24, lanes 7-11). Not only were these capsid proteins expressed, they were also assembling into particles, likely to be chimaeric AHSV-1/AHSV-7 VLPs, within the 55-35% sucrose fractions. The presence of the AHSV-1 VP3, VP7, VP5 capsid proteins was also confirmed in the 55-35% sucrose fractions (FIG. 24, lanes 2-6) indicating particle self-assembly. Although the AHSV-1 neutralization-specific VP2 antigen could not be detected with the AHSV-7-specific antiserum, its presence cannot be ruled out. The identity of the AHSV-1 VP3, AHSV-1 VP7, AHSV-1 VP5, AHSV-7 VP5 proteins was confirmed Mass Spectrometry (MS) analysis (Dr Stoyan Stoychev, CSIR Biosciences). The identity of the AHSV-1 VP2 and AHSV-7 VP2 proteins has not yet been confirmed via MS analysis.

It was hypothesized that a double chimaeric VLP particle, where the VP2 and VP5 outer capsid proteins originate from one serotype and the core proteins VP7 and VP3 from another serotype of AHSV, may be more stable than the single chimaeric VLP where only the outer capsid protein VP2 is exchanged. N. benthamiana leaves were hence infiltrated with combinations of recombinant Agrobacterium tumefaciens bacteria containing the AHSV-1 VP3, AHSV-1 VP7, AHSV-1 VP5 or AHSV-7 VP5 and AHSV-7 VP2 constructs and harvested 8 dpi. Sucrose gradient centrifugation of the leaf extracts and immunoblotting of the resulting sucrose fractions with guinea pig αAHSV-7 serum indicated a greater quantity of the AHSV-7 VP2 and VP5 proteins in fractions 55-50% (FIG. 25, lanes 8-9) than the AHSV-7 VP2 and AHSV-1 VP5 proteins in the same fractions of their respective sucrose gradient (FIG. 25, lanes 6-7). The quantity of the AHSV-1 VP7 and VP3 remained relatively constant in these fractions from the different sucrose gradients. This may be indicative of a larger quantity of VP2 and VP5 proteins on their outer shells of the putative double chimaeric VLPs (FIG. 25, lanes 8-9) when compared to the putative single chimaeric VLPs (FIG. 25, lanes 6-7). Presenting both outer capsid proteins from serotype 7 on a scaffold of AHSV-1 core proteins may thus help to further stabilise the proposed chimaeric AHSV-1/AHSV-7 VLP particle. In order to facilitate the assembly of chimaeric AHSV-1 based VLPs presenting the outer capsid proteins of the remaining AHSV serotypes, a double recombinant pEAQ-express-AHSV-1VP7-AHSV-1VP3 expression vector was constructed.

In order to investigate whether it may be possible to assemble a triple chimaeric AHSV VLP particle in plants, where the origin of the capsid proteins is from three different AHSV serotypes, constucts encoding the AHSV-1 VP7/AHSV-1 VP3, AHSV-3 VP5 and AHSV-6 VP2 proteins were infiltrated into Nicotiana benthamiana dXT-FT plant leaves. The leaves were harvested 8 days post-infiltration and the cell extracts centrifuged through 70-30% sucrose gradient. Sucrose fractions were electrophorized on precast denaturing 4-12% Bolt™ Bis-Tris Plus polyacrylamide Gels (Thermo Fischer Scientific™), as described above, and candidate protein bands excised from the gel and sent for Mass spectrometry (MS) analysis (Table 5). The large number of AHSV-6 VP2-specific peptides confirm the assembly of the triple chimaeric AHSV-1/AHSV-3/AHSV-6 VLPs in N. benthamiana dXT-FT plant cells.

TABLE 5
Mass Spectrometry (MS) results of triple chimaeric
AHSV-1/AHSV-3/AHSV-6 combination in plants

| Protein | | | Peptides |
|---|---|---|---|
| Band | % Cov (95) | Name | (95%) |
| 1 | 18.95 | AHSV-6 VP2 plant codon optimised | 18 |
| 2 | 32.86 | AHSV-6 VP2 plant codon optimised | 35 |

VLPs and/or CLPs were visualised by adsorbing samples from 55% sucrose fractions onto carbon-coated holey copper grids as follows: The grids were floated on the protein sample for 30 seconds, the excess sample drained off the grid via blotting on filter paper and the grid then floated on 2% sodium phosphotungstate, pH 7 for 30 sec. The excess stain was drained off by blotting the grid onto filter paper. The grid was air dried and subsequently imaged in a JEM-2100 Transmission electron microscope (JEOL™). The diameters of the particles visualised on the grid were measured using the measure tool on the Gatan Digital Micrograph™ software. Thirty five particles of each type were measured and the mean diameter calculated.

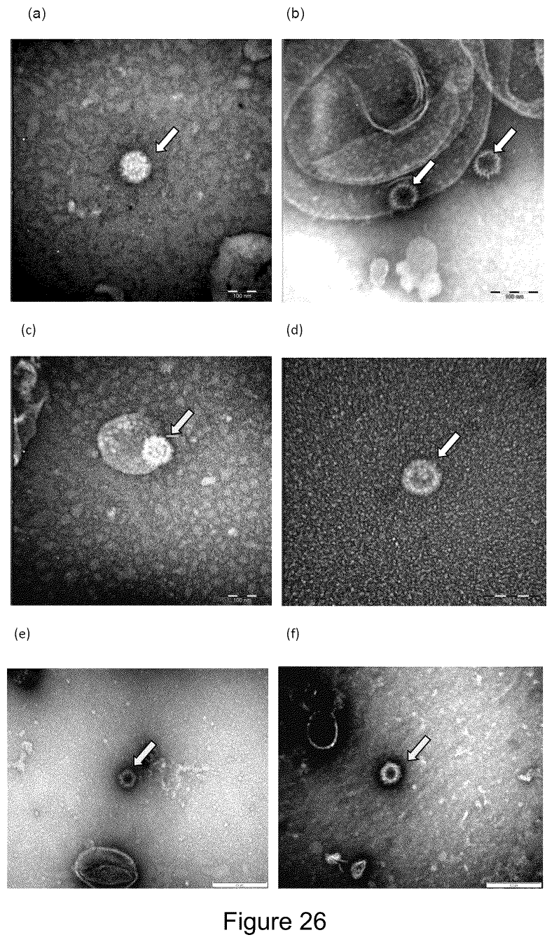

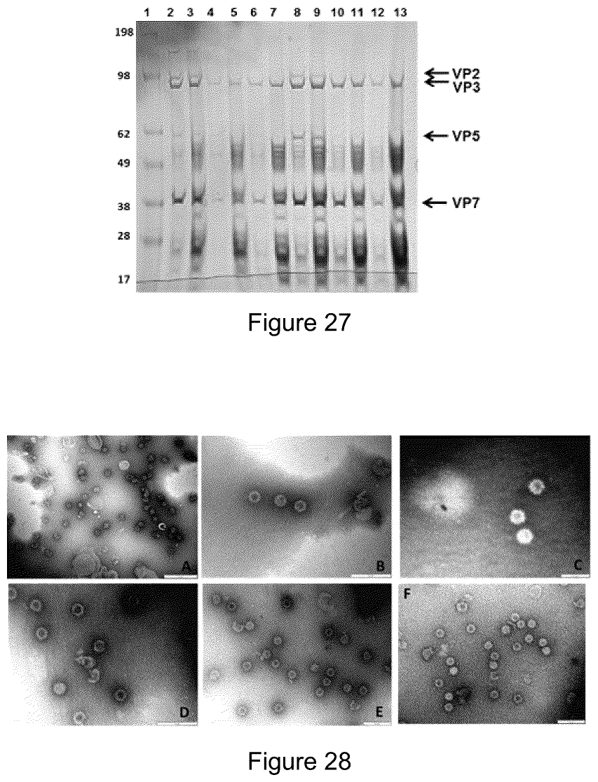

Transmission electron microscope (TEM) viewing of the particles present in the 55% sucrose gradient fractions of the gradients depicted above, as well as a gradient of the AHSV-1VP7-AHSV-1VP3 plant cell lysate, indicated the presence of core-like particles (CLPs) and virus-like particles (VLPs) (FIG. 26). The transiently expressed AHSV-1 VP2, VP5, VP3 and VP7 capsid proteins self-assembled into VLPs in N. benthamiana plant cells (FIG. 26(a)). These AHSV-1 VLPs were approximately 70 nm in diameter and appeared more fuzzy and dense than the ‘spiky’ 60 nm AHSV-1 core like particles (CLPs), consisting only of the VP7 and VP3 core proteins (FIG. 26(b)). The AHSV-1 VP3, VP7, AHSV-1 or AHSV-7 VP5 and AHSV-7 VP2 proteins also self-assembled into either single or double chimaeric AHSV-1/AHSV-7 VLPs (FIG. 3, (c) and (d), respectively), which were also approximately 70 nm in diameter. The triple chimaeric AHSV-1/AHSV-3/AHSV-6 VLPs can be visualised in FIG. 26 (e)-(f). They were also approximately 70-75 nm in size. The diameters of AHSV virion particles have previously been described as approximately 70 nm in diameter (Coetzer & Guthrie, 2004).

In the present Example the inventors have successfully produced the first documented African horse sickness virus-like particles in Nicotiana benthamiana or Nicotiana benthamiana dXT-FT plants. These VLPs, based on AHSV-1, will be used as a component of a multivalent vaccine against the nine African horse sickness serotypes. In addition, the inventors have also succeeded in generating single and double chimaeric AHSV-1/AHSV-7 VLPs, as well as triple chimaeric AHSV-1/AHSV-3/AHSV-6 VLPs in plants. In this case, particles, formed from the AHSV-1 VP3 and VP7 capsid proteins, function as a scaffold for the presentation of the entire VP2 and/or VP5 antigen of other AHSV serotypes to the immune system. An alternative scaffold, created from the capsid proteins of any one or more of the remaining eight AHSV serotypes, is not excluded. The AHSV VLP-based presentation system is in the process of being developed by the inventors for the presentation of all nine AHSV neutralization VP2 antigens to serve as an efficacious, multivalent vaccine against African horsesickness. An initial target animal trial, described in Example 8, has been conducted and preliminary data indicate that plant-expressed, double chimaeric AHSV-1/AHSV-7 VLPs are immunogenic in horses. A second target animal immunogencity trial with a triple chimaeric AHSV-1/AHSV-3/AHSV-6 VLP particle is currently underway to confirm these results.

### Example 5

Plant codon optimised nucleotide sequences were synthesised by Bio Basic Int™. Both the nucleotide sequences and the proteins that they encode are described in Example 3. Using the protocols described herein the nucleotide sequences were cloned into the pEAQ-HT expression vectors. In this experiment Agrobacterium harbouring the pEAQ-HT with inserts were taken from a seed cell bank. The aim of this experiment was to compare the stable assembly of double and single chimaeric VLPs of serotypes BTV-4 and BTV-3 using BTV-8 core proteins; and also the most appropriate combinations to result in stable chimaeric VLPs.

Transient expression efficiency of the pEAQ series of vectors was investigated by agroinfiltration of Nicotiana benthamiana which facilitates mammalian-like or human-like glycosylation RNAi mutant dXT/FT. The pEAQ-HT constructs containing genes encoding individual capsid proteins of BTV serotypes 3, 4 and 8 were stored as seed cell banks. Prior to plant infiltration Agrobacterium tumefaciens strain (LBA4404) transformed with pEAQ-HT vector containing individual VP2, VP3, VP5 and VP7 of selected serotypes were streaked on YMB agar plates and incubated at 28° C. for 48 hrs. The growing bacterium was scraped off from the plate and inoculated into YMB broth with the relevant antibiotics and grown overnight. Cells were pelleted and resuspended in MMA buffer (100 mM MES, 10 mM MgCl₂ and 100 mM acetosyrigone; pH 5.6). Each of the four Agrobacterium cultures was adjusted to OD₆₀₀ of approximately 0.5 with the same buffer. The formation of VLPs was validated by mixing and infiltrating the four constructs encoding the four individual capsid proteins at a ratio of 1:1:1:1 and used for plant infiltrations (5 plants per construct combination; 15-20 cm in height).

The leaf material was harvested eight days after infiltration using a Matstone Multipurpose™ juice extractor in VLP extraction buffer (50 mM bicine, pH 8.4; 20 mM sodium chloride [NaCl], 0.1% (w/v) N-lauroylsarcosine (NLS) sodium salt; 1 mM dithiothreitol (DTT)) in a ratio 1:3 with complete protease inhibitor cocktail (P2714, Sigma® Life Sciences) added to the VLP extraction buffer immediately before the extraction started. Crude extracts were centrifuged twice for 10 minutes at 4,200×g, 10° C. to remove cell debris in a JA14 rotor using a Beckman Coulter™ Avanti J-26 XPI centrifuge.

Particles were purified by density gradient centrifugation using ultra-high quality sucrose (Sigma® Life Sciences) step gradients (30%-70%) prepared dissolved in VLP dilution buffer (50 mM Bicine, pH 8.4, 20 mM NaCl). Step gradients of 1 ml with 10% incrementing steps were prepared and then overlaid with 8 ml of clarified leaf extract. The gradients were centrifuged at 85,800×g, at 10° C. for 3 hours in a SW-41Ti rotor (Beckman Coulter®). Sucrose gradient fractions (45%-50%) were collected and aliquots (26 μl) were analysed on a 4-12% Bis-Tris Bolt™ (Life Technologies®) protein gel. Distinct protein bands were identified on Coommassie stained gels. The protein bands at 111 kDa, 100 kDa, 59 kDa and 38 kDa were confirmed to be the capsid proteins VP2, VP3, VP5 and VP7, respectively, by mass spectrophotometry. The assembly of VLPs (˜70 nm) was confirmed by transmission electron microscopy (TEM) for all three serotypes 3, 4 and 8 (FIG. 27).

The sucrose gradient fractions were adsorbed onto holey carbon-coated copper grids as follows. The grids were floated on the 1/10 dilution protein sample for 30 seconds and excess sample drained off the grid via blotting on filter paper. Subsequently the grid was floated on 2% sodium phophotungstate, pH 7.0 for 30 seconds (0.22 μm filter sterilized before staining) and drained as described above (FIG. 28).

Protein bands of interest were in-gel trypsin digested as per the protocol described in Example 3. Protein pilot v5 using Paragon search engine (AB Sciex™) was used for comparison of the obtained MS/MS spectra with Uniprot Swissprot protein database. Proteins with threshold above ≥99.9% confidence were reported (Table 6).

TABLE 6
Combinations of capsid proteins and peptides identified by Mass
Spectrometry.
Bluetongue virus BTV-4, BTV-3 & BTV-8 seed cell bank

| Sample # | | Protein Size | Peptides | 95% coverage |
|---|---|---|---|---|
| BTV-8 homogenous VLPs | VP2 | 111 kDa | 150 | 67.6% |
| BTV-3 single chimaeric (BTV-8 VP3, | VP2 | 111 kDa | 22 | 31.0% |
| VP5 & VP7, BTV-3 VP2) | | | | |
| BTV-3 double chimaeric (BTV-8 VP3 | VP2 | 111 kDa | 81 | 62.8% |
| & VP7, BTV-3 VP2 & VPS) | | | | |
| BTV-4 single chimaeric (BTV-8 VP3, | VP2 | 111 kDa | 18 | 29.3% |
| VP5 & VP7, BTV-4 VP2) | | | | |
| BTV-4 double chimaeric (BTV-8 VP3 | VP2 | 111 kDa | 48 | 54.0% |
| & VP7, BTV-4 VP2 & VPS) | | | | |
| BTV-4 single chimaeric (BTV-8 VP3, | VP2 | 111 kDa | 42 | 50.4% |
| BTV-4 VP2, VPS & VP7) | | | | |

Since all the Agrobacterium cultures were prepared from the same seed cell bank, infiltrated in the same batch of plants, extracted with the same extraction buffer, subjected to ultracentrifugation in the same run and equal amounts were loaded on the same SDS PAGE 4-12% Bolt™ precast gel, we confidently make the assumption that double chimaerics have more VP2 protein assembled. The results indicate that the assembly of double chimaeric (both outer capsid proteins) VLPs is superior to the assembly of single chimaeric (only VP2 outer capsid substituted) VLPs. Almost four times more peptides of VP2 were detected when the VLPs of BTV-3 was assembled via double chimaeric versus single chimaeric combinations, 81 versus 22 peptides, respectively. Similarly, almost three times more VP2 peptides were detected when the VLPs of BTV-4 was assembled via double chimaeric versus single chimaeric combinations, 48 versus 18 peptides, respectively. For both scenarios above, single chimaeric indicates that BTV-8 core (VP3, VP7 and VP5) was combined with VP2 from a second serotype. When BTV-8 VP3 was combined with the remaining capsid proteins VP2, VP7 and VP5 of serotype 4, 42 peptides were detected, 6 peptides less than BTV-4 double chimaeric VLP combination. Nevertheless, all animals trials were conducted with BTV-4 and BTV-3 double chimaeric VLP vaccines. Homogenous BTV-8 VLPs resulted in 150 VP2 peptides.

### Combinations of BTV-3 Single and Double Chimaeric VLPs

BTV-3 single chimaerics was assembled by proteins BTV-8 VP3 and BTV-8 VP7 forming CLPs combined with BTV-8 VP5 and BTV-3 VP2. BTV-3 double chimaerics was assembled by proteins BTV-8 VP3, BTV-8 VP7 combined with BTV-3 VP2, BTV-3 VP5.

### Combinations of BTV-4 Single and Double Chimaeric VLPs

BTV-4 single chimaerics was assembled by proteins either of 1). BTV-8 VP3 and BTV-8 VP7 forming CLPs combined with BTV-8 VP5 and BTV-4 VP2 (only VP2 of serotype 4) or 2). BTV-8 VP3 combined with BTV-4 VP7, VP5 and VP2 (only VP3 of serotype 8). BTV-4 double chimaerics was assembled by proteins BTV-8 VP3, BTV-8 VP7 combined with BTV-4 VP2, BTV-4 VP5.

### Example 6

### BTV-4 Double Chimaeric VLP Extraction and Purification for Sheep Trial

A large scale VLP purification system was established for biomass BTV4 VLP production for the purpose of subsequent target animal (sheep) immunogenicity studies. Hand infiltration of Agrobacterium harbouring BTV serotype 8 and 4 genes encoding the four capsid proteins (BTV-8 VP3 and VP7; BTV-4 VP2 and VP5) was conducted as described in Example 3. Thirty to forty plants were infiltrated with the Agrobacterium culture. Once more the leaf material was harvested eight days after infiltration in Bicine buffer. Remaining plant debris was removed by filtering the cell lysate through two layers of Miracloth™ before two successive centrifugations steps (4200×g for 10 minutes each at 10° C.). The plant extract was then filtered through a Sartoclean GF™ sterile midicap (3 μM+8 μ) using a Masterflex Console Drive peristaltic pump (Cole-Parmer Instrument Company™). To further purify, the lysate was filtered through a 300K Minimate™ Tangential Flow Filtration (TFF) Capsule (Pall Life Sciences™) with the pressure not exceeding 2 Bar. The latter removes all proteins smaller than 300K. The NLS detergent, DTT and protease inhibitor was removed from the VLP containing extract through two subsequent wash steps (1 in 10 dilution each) with sterile VLP dilution buffer. D-(+)-Trehalose dihydrate (Sigma® Life Science) (5% m/v) was added to the extract (50 ml) to stabilise the VLP extract. The extract was filter sterilised through a 0.45 μM+0.2 μM Sartobran 300 sterile capsule (Sartorius Stedim biotech GmbH™) using a peristaltic pump.

In addition to TFF purification, a fraction of the crude plant lysate was also purified with sucrose gradient centrifugation. The lysate (23 ml) was layered on top of sucrose density gradients (70-30%; 3 ml each) and centrifuged at 85,800×g, at 10° C. for 3 hours in a SW-32Ti rotor (Beckman Coulter®) in 38.6 ml volume ultra-clear Beckman® tubes. The first 6 ml was discarded (60-70% fractions) and the following 6ml (50-40%) containing the VLPs, was collected. The sucrose-gradient purified product was dialysed overnight against Bicine buffer containing only the Bicine (pH 8.4) and sodium chloride before filter sterilization in preparation for animal trials. The TFF and sucrose fractions used for the animal trial was mixed (1:1) with Alhydrogel and transported to Onderstepoort Biological Products™ (OBP) on ice. The vaccine was administered on the day of delivery at OBP.

The sheep trial was conducted according to the procedures and schedule detailed in the target animal ethics application submitted to the Animal Ethics Committee OBP. Approval was subsequently obtained from CSIR Research Ethics Committee. In short, sheep were stabled and handled according to standard operating procedures outlined by the Experimental unit. Vaccination and bleeding of animals was according to standard practises. Animals were bled on days 0, 7, 14, 21, 28, 35, 42, 49 and 56. The primary vaccine was administered on day 0 and 21 with 500 μl sterile purified BTV-4 VLPs and 500 μl Alhydrogel. Sheep 554 and 513 were vaccinated with TFF purified VLPs, sheep 521, 566 and 656 with sucrose gradient purified VLPs, sheep 551 with live attenuated BTV-4 antigens (positive control) and sheep 634 with Bicine buffer alone to serve as negative control.

Serum neutralizing tests (SNTs) were conducted to determine antibody titers and used to demonstrate seroconversion (Table 7). A titer of ≥1:4 will demonstrate seroconversion. Seroconversion was shown for the control sheep, three sucrose gradient and one TFF vaccinated animals. Sheep 554 was inadvertently pre-exposed to BTV.

TABLE 7
Serum neutralizing test (SNT) results of the sheep trial.

| Sheep | | Day | | | | | | | | |
|---|---|---|---|---|---|---|---|---|---|---|
| # | Innoculum | 0 | D 7 | D 14 | D 21 | D 28 | D 35 | D 42 | D 49 | D 56 |
| 554 | TFF* | 0 | 0 | 0 | 0 | 0 | 0 | 0 | 0 | 0 |
| 513 | TFF | 0 | 0 | 1:4 | 0 | 0 | 0 | 1:8 | 0 | 0 |
| 521 | Sucrose | 0 | 0 | 1:4 | 1:128 | 1:128 | 1:256 | 1:256 | 1:256 | 1:256 |
| 566 | Sucrose | 0 | 0 | 0 | 1:8 | 1:32 | 1:256 | 1:256 | 1:256 | 1:256 |
| 656 | Sucrose | 0 | 0 | 0 | 0 | 1:2 | 1:32 | 1:32 | 1:32 | 1:64 |
| 551 | OBP live attenuated | 0 | 0 | 0 | 1:16 | 1:128 | 1:128 | 1:256 | 1:256 | 1:256 |
| | BTV-4 virus | | | | | | | | | |
| | (Positive control) | | | | | | | | | |
| 634 | Bicine buffer | 0 | 0 | 0 | 0 | 0 | 0 | 0 | 0 | 0 |
| | (Negative control) | | | | | | | | | |

### Example 7

### BTV-3 VLP Extraction and Purification for Sheep Trial

A large scale VLP purification system was established for biomass BTV-3 VLP production for the purpose of subsequent target animal (sheep) immunogenicity studies. Hand infiltration of Agrobacterium harbouring BTV serotype 8 and 3 genes encoding the four capsid proteins (BTV-8 VP3 and VP7; BTV-3 VP2 and VP5) was conducted as described in Example 3. Thirty to forty plants were infiltrated with the LBA4404 Agrobacterium culture harbouring the pEAQ-HT vector and genes encoding the capsid proteins described above. Once more the leaf material was harvested eight days after infiltration in Bicine buffer. Remaining plant debris was removed by filtering the cell lysate through two layers of miracloth before two successive centrifugations steps (4200×g for 10 minutes each at 10° C.). The plant extract was then filtered through a Sartoclean™ GF sterile midicap (3 μM+8 M) using a Masterflex Console Drive™ peristaltic pump (Cole-Parmer Instrument Company™). To further purify, the lysate was filtered through a 300K Minimate™ Tangential Flow Filtration (TFF) Capsule (Pall Life Sciences™) with the pressure not exceeding 2 Bar. The latter removes all proteins smaller than 300K. The NLS detergent, DTT and protease inhibitor was removed from the VLP containing extract through two subsequent wash steps (1 in 10 dilution each) with sterile VLP dilution buffer. D-(+)-Trehalose dihydrate (Sigma® Life Science) (5% m/v) was added to the extract (50 ml) to stabilise the VLP extract. The extract was filter sterilised through a 0.45 μM+0.2 μM Sartobran 300 sterile capsule (Sartorius Stedim biotech GmbH™) using a peristaltic pump.

In addition to TFF purification, a fraction of the crude plant lysate was also purified with sucrose gradient centrifugation. The lysate (23 ml) was layered on top of sucrose density gradients (70-30%; 3 ml each) and centrifuged at 85,800×g, at 10° C. for 2 hours in a SW-32Ti rotor (Beckman Coulter®) in 38.6 ml volume ultra-clear Beckman® tubes. The first 6.5 ml was discarded (60-70% fractions) and the following 3 ml (50-40%) containing the VLPs, was collected. The sucrose-gradient purified product was dialysed overnight against phosphate buffer (pH 7.4) before filter sterilization in preparation for animal trials. The TFF and sucrose fractions used for the animal trial was mixed (1:1) with Montanide™ ISA 201 VG and transported to Onderstepoort Biological Products™ (OBP) on ice. The vaccine was administered on the day of delivery at OBP.

Sheep were stabled and handled according to standard operating procedures outlined by the Experimental unit at OBP. Vaccination and bleeding of animals was according to standard practises. Animals were bled on days 0, 7, 14, 21, 28, 35 and 42. The primary vaccine was administered on day 0 and 21 with 500 μl sterile purified BTV-3 VLPs and 500 μl Montanide™ ISA 201 VG. Sheep 1646, 1639 and 1655 were vaccinated with TFF purified VLPs, sheep 1657, 1605 and 1613 with sucrose gradient purified VLPs, sheep 1632, 1647 and 1609 with sucrose gradient omitting the adjuvant; sheep 1608 and 1614 with live attenuated BTV-3 antigens (positive control) and sheep 1649 with Bicine buffer alone and sheep 1629 naïve, untouched to serve as negative control.

Serum neutralizing tests (SNTs) were conducted to determine antibody titers and used to demonstrate seroconversion. A titer of ≥1:4 will demonstrate seroconversion. Seroconversion was shown for all three TFF vaccinated animals identical to live BTV-3 monovalent vaccinations (Table 8).

TABLE 8
Serum neutralising test (SNT) results of the sheep trial.

| | | Day | | | | | | | | |
|---|---|---|---|---|---|---|---|---|---|---|
| | Pre-bleed | 0 | D 7 | D 14 | D 21 | D 28 | D 35 | D 42 | D 49 | D 56 |
| TFF (ISA 201) | | | | | | | | | | |
| 1646 | — | — | — | — | 2 | 256 | 256 | 256 | 256 | 256 |
| 1639 | — | — | — | — | 2 | 256 | 256 | 256 | 256 | 256 |
| 1655 | — | — | — | — | 4 | 256 | 256 | 256 | 256 | 256 |
| Sucrose (ISA 201) | | | | | | | | | | |
| 1657 | — | — | — | — | — | — | 2 | 16 | 16 | — |
| 1605 | — | — | — | — | — | 2 | 16 | — | 2 | — |
| 1613 | — | — | — | — | — | — | — | — | — | — |
| Sucrose (no | | | | | | | | | | |
| adjuvant) | | | | | | | | | | |
| 1632 | — | — | — | — | — | — | 8 | 8 | — | — |
| 1647 | — | — | — | — | 2 | 2 | — | 4 | — | — |
| 1609 | — | — | — | — | 4 | 8 | — | — | — | — |
| Live monovalent | | | | | | | | | | |
| 1608 | — | — | 128 | 256 | 256 | 256 | 256 | 256 | 256 | 256 |
| 1614 | — | — | — | 128 | Δ | 256 | 256 | 256 | 256 | 256 |
| Controls | | | | | | | | | | |
| Bicine buffer | | | | | | | | | | |
| 1649 | — | — | — | — | — | — | — | — | — | — |
| Naïve, untouched | | | | | | | | | | |
| 1629 | — | — | — | — | — | — | — | — | — | — |

### Example 8

### VLP Purification and Immunogenicity Trial of Double Chimaeric AHSV VLPs in Horses

Following agroinfiltration of Nicotiana benthamiana dXT-FT plants with the appropriate recombinant pEAQ vectors, the double chimaeric AHSV-1/AHSV-7 VLPs were purified by means of both tangential flow filtration (TFF) and sucrose density gradient centrifugation prior to being injected into horses. More specifically, LBA 4404 agrobacterial cells, containing the recombinant plasmids PEAQ-express-AHSV-1VP7/AHSV-1VP3, PEAQ-HT-AHSV-7VP5 and PEAQ-HT-AHSV-7VP2 were defrosted and streaked out onto selective LB plates (50 μg/ml Kanamycin, 50 μg/ml Rifampicin and 50 μg/ml Streptomycin). Following incubation at 28° C. for 48 hours, the cultures were subsequently inoculated into 50 ml YMB medium containing the appropriate antibiotics and incubated overnight at 28° C. with rotational shaking (175 rpm). The overnight cultures were harvested at 8000 rpm for 7 min at 20° C. and each cell pellet resuspended in 40 ml MMA buffer (10 mM MES hydrate; pH 5.6, 10 mM MgCl₂, 100 μM 3,5-Dimethoxy-4-hydroxy-acetophenone) and the OD₆₀₀ measured. The agrobacterial suspensions were combined in a 1:1:1 ratio and the final OD₆₀₀ of each combination was 0.4-0.5. Four week-old N. benthamiana dXT-FT plants were infiltrated with the agrobacterial combination via syringe-mediated infiltration.

The infiltrated leaves were harvested 8 days post infiltration (d.p.i), weighed and immediately processed through a juice extractor (MATSTONE 6™ in 1 multipurpouse juice extractor) with 3 volumes of VLP extraction buffer (20 mM NaCl, 50 mM Bicine, pH 8.4, 0.1% (w/v) Sodium lauroyl sarcosine (NLS), 1 mM Dithiothreitol (DTT) (ThermoScientific™), complete, EDTA-free Protease inhibitor cocktail (Sigma-Aldrich®)). The DTT and complete, EDTA-free Protease inhibitor cocktail tablets were freshly prepared according to the manufacturers' instructions and added to the extraction buffer just prior to use. Large plant debris was removed by filtering the cell lysate through 2 layers of miracloth and the cell extract clarified via low speed centrifugation (4200×g; 30 min; 10° C.). Using a Masterflex Consol Drive peristaltic pump (Cole-Parmer Instrument Company™), the cell extract was filtered through a Sartoclean GFT sterile midicap (3+0.8 μM) depth filter (Sartorius™).

A portion of the filtrate was layered on top of 70-30% sucrose gradients and centrifuged in a SW-38Ti rotor (Beckman Coulter®) at 85,800×g for 3 hours; 10° C. Sucrose solutions (30%-70%) were prepared by dissolving ultra-high quality sucrose (Sigma® Life Science) in VLP dilution buffer (20 mM NaCl, 50 mM Bicine, pH 8.4) and layered into gradients of 3 ml 10% incrementing steps. Following centrifugation the 55%-45% sucrose layers were harvested in 1 ml fractions via a Minipuls2™ peristaltic pump (Gilson™). Fractions containing the VLPs (55%-45%) were added together and dialysed against sterile VLP dilution buffer (20 mM NaCl, 50 mM Bicine, pH 8.4) overnight, with gentle stirring, in SnakeSkin™ dialysis tubing (Thermo Fisher Scientific®). This was followed by a second dialysis step of 2 hours against new VLP dilution buffer at 4° C. The dialysed sample was harvested and D-(+)-Trehalose dihydrate (Sigma® Life science) added as a stabilizing agent to a final concentration of 5%.

The remainder of the Depth filtered plant cell extract was further filtered through a 300K Minimate™ Tangential Flow filtration (TFF) Capsule (Pall Life Sciences™) with the pressure not exceeding 2 Bar. This was done to remove all proteins smaller than 300K. Two subsequent wash steps (1 in 10 dilution each) with sterile VLP dilution buffer ensured the removal of the NLS detergent, DTT and Protease inhibitor from the plant extract. The plant cell lysate was concentrated to ⅕ of its original volume and D-(+)-Trehalose dihydrate (Sigma® Life science) added as a stabilizing agent to a final concentration of 5%.

The sucrose and TFF purified samples were subsequently filter-sterilized through a 0.45 μM+0.2 μM Sartobran™ 300 Sterile capsule (Sartorius Stedim biotech GmbH™) utilizing a peristaltic pump with the pressure not exceeding 2 Bar. They were also tested for sterility by streaking out 100 μl of the sample on Luria agar plates containing no antibiotics and incubating those plate overnight at 37° C. Samples, taken throughout the course of the purification procedure, were analysed for protein content by denaturing SDS-PAGE and immunoblotting procedures with AHSV-7 specific antiserum, kindly donated by OBP. The protein content of the filter sterilized samples was quantified by using the Micro BCA™ Protein Assay kit (Thermo Fisher Scientific®) while the VLPs in these same samples was visualised via TEM.

The immunogenicity of the plant-produced double chimaeric AHSV-1/AHSV-7 VLPs was investigated in the target species, horses. The horse trial was conducted according to the procedures and schedule detailed in the approved target animal ethics applications (CSIR REC registration number 151/2015, OBP registration number 2015/003). Seven AHS-naïve foals (6 months old) were stabled in closed stables at OBP and handled according to standard operating procedures outlined by the Experimental Unit. Vaccination and bleeding of animals was according to standard operating protocols and conducted by OBP. Three foals were each injected subcutaneously into the inner thigh with the TFF-purified VLP/Alydrogel® sample (final volume of 2 ml containing 3490 μg of total protein). Two foals were each injected subcutaneously into the inner thigh with the sucrose gradient-purified VLP/Alydrogel® sample (final volume of 2 ml containing 101 μg of total protein). One foal was inoculated with sterile bicine buffer/Alydrogel® sample as a negative control whilst another was inoculated with monovalent AHSV-7 live attenuated vaccine (OBP) as a positive control. The animals were inoculated with the booster sample on day 28 of the immunization schedule. The 2 ml TFF-purified VLP/Alydrogel® booster sample contained 3825 μg of total protein while the 2 ml sucrose gradient-purified VLP/Alydrogel® sample contained 184 μg of total protein. The two control animals received sterile bicine buffer/Alydrogel® and monovalent AHSV-7 live attenuated vaccine (OBP), respectively, during the boost inoculation. Serum samples were taken on days 0, 7, 14, 21, 28, 35, 42, 49 and 56. Serum neutralization testing was performed on the blood samples by OBP according to the RDV-ME-014 method whilst the VP7-specific ELISA tests were performed by ARC Onderstepoort Veterinary Institute (OVI).

The results of this horse trial are as follows: One horse (#31), inoculated with the TFF-purified AHSV-1/AHSV-7 VLP sample, elicited α-AHSV-7 neutralizing antibodies with a titre of 1:16 two weeks after the boost inoculation (day 42) (Table 9 & 10). This indicates that the AHSV-7 VP2 protein was presented on the surface of the double chimaeric VLPs in a conformation capable of eliciting an AHSV neutralizing humoral immune response in the horse. However, this immune response was not detected in the following two weeks (days 49 and 56). No neutralizing antibodies were detected in the sera of any of the other animals during the trial, not even the animals injected with the monovalent live attenuated AHSV-7 virus used as a positive control (Animal #32). The lack of a response in the positive control group indicates that this trial will have to be repeated. Horses #29 and #30, both inoculated with the TFF-purified AHSV-1/AHSV-7 VLPs, as well as horse #35, inoculated with sucrose gradient purified AHSV-1/AHSV-7 VLPs, elicited antibodies against the VP7 protein on day 35, a week after the booster inoculation. This indicates the presence of CLPs. As expected, the animal inoculated with bicine buffer, #43, did not elicit any neutralizing or VP7-specific antibodies during the course of the trial.

TABLE 9
Serum neutralizing test (SNT) results of the horse trial.

| Horse | | Day | | | | | | | | |
|---|---|---|---|---|---|---|---|---|---|---|
| # | Innoculum | 0 | D 7 | D 14 | D 21 | D 28 | D 35 | D 42 | D 49 | D 56 |
| 23 | TFF-purified AHSV-1/7 | 0 | 0 | 0 | 0 | 0 | 0 | 0 | 0 | 0 |
| | VLPs | | | | | | | | | |
| 30 | TFF-purified AHSV-1/7 | 0 | 0 | 0 | 0 | 0 | 0 | 0 | 0 | 0 |
| | VLPs | | | | | | | | | |
| 31 | TFF-purified AHSV-1/7 | 0 | 0 | 0 | 0 | 0 | 0 | 1:16 | 0 | 0 |
| | VLPs | | | | | | | | | |
| 35 | Sucrose gradient purified | 0 | 0 | 0 | 0 | 0 | 0 | 0 | 0 | 0 |
| | AHSV-1/7 VLPs | | | | | | | | | |
| 41 | Sucrose gradient purified | 0 | 0 | 0 | 0 | 0 | 0 | 0 | 0 | 0 |
| | AHSV-1/7 VLPs | | | | | | | | | |
| 32 | Monovalent live | | | | | | | | | |
| | attenuated AHSV-7 OBP | 0 | 0 | 0 | 0 | 0 | 0 | 0 | 0 | 0 |
| | (Positive control) | | | | | | | | | |
| 43 | Bicine buffer (Negative | 0 | 0 | 0 | 0 | 0 | 0 | 0 | 0 | 0 |
| | control) | | | | | | | | | |

TABLE 10
ELISA results of the horse trial.

| Horse | | | | | |
|---|---|---|---|---|---|
| # | Innoculum | D 14 | D 21 | D 28 | D 35 |
| 23 | TFF-purified AHSV-1/7 | Neg | Neg | Neg | 9 |
| | VLPs | | | | |
| 30 | TFF-purified AHSV-1/7 | Neg | Neg | Neg | 14 |
| | VLPs | | | | |
| 31 | TFF-purified AHSV-1/7 | Neg | Neg | Neg | Neg |
| | VLPs | | | | |
| 35 | Sucrose gradient purified | Neg | Neg | Neg | 22 |
| | AHSV-1/7 VLPs | | | | |
| 41 | Sucrose gradient purified | Neg | Neg | Neg | Neg |
| | AHSV-1/7 VLPs | | | | |
| 32 | Monovalent live | Neg | Neg | Neg | Neg |
| | attenuated AHSV-7 OBP | | | | |
| | (Positive control) | | | | |
| 43 | Bicine buffer (Negative | Neg | Neg | Neg | Neg |
| | control) | | | | |

### Example 9

### Immunogenicity of Plant Produced African Horse Sickness Virus-Like Particles

A consensus gene sequence for each of the AHSV-5 viral capsid proteins VP2, VP3, VP5 and VP7 was obtained by aligning all the known sequences for these genes listed in GenBank™, using CLC Mainbench bioinformatics software (Qiagen Bioinformatics™, Aarhus, Denmark). Consensus sequences were codon optimized for expression in N. benthamiana and synthesized by GenScript™ Biotech Corporation (China) with flanking AgeI and XhoI restriction enzyme sites. The codon-optimized VP7 consensus sequence, modified as described by S. Bekker (2015) to include 7 amino acid substitutions near the 3′ end, (Pro276His, Arg328Ala, Val333Asn, Ala334Pro, Pro335Met, Val336Pro and Gln338Pro) was also synthesized. Restriction enzyme cloning was used to insert the genes into the pEAQ-HT expression vector obtained from George Lomonossoff, John Innes Centre, UK (Sainsbury et al., 2009) to produce PEAQ-AHS5-VP2, pEAQ-AHS5-VP3, PEAQ-AHS5-VP5, PEAQ-AHS5-VP7 and PEAQ-AHS5-VP7mu. The AHSV-5 plasmid constructs were electroporated into Agrobacterium radiobacter AGL1-ATCC BAA-101 as described previously (Maclean et al., 2007) and recombinant clones were selected at 27° C. on Luria Bertani (LB) media plates containing 25 μg/mL carbenicillin and 50 μg/mL kanamycin.

### Transient Expression in Plants

Expression of the AHSV-5 capsid proteins was achieved by agroinfiltration of 5-6-week-old N. benthamiana plants. Agrobacterium transformants each carrying one of the AHSV-5 capsid protein genes, were subcultured and grown overnight with agitation at 27° C. in Luria Bertani Broth (LBB) base supplemented with 50 μg/mL kanamycin, 20 μM acetosyringone and 2 mM MgSO₄. The cultures were diluted in resuspension solution (10 mM MES, pH 5.6, 10 mM MgCl₂, 100 μM acetosyringone) to the desired optical density and incubated for 1 h at 22° C. to allow for expression of the vir genes. For single infiltrations, each AHSV-5 Agrobacterium recombinant suspension was diluted to OD₆₀₀=0.5 or 1.0, while co-infiltration suspensions contained all four AHSV-5 recombinants in a ratio VP2:VP3:VP5:VP7 of 1:1:1:1 or 1:1:2:1. Plants were grown at 22-25° C. under 16 h/8 h light/dark cycles. Agrobacterium suspensions were infiltrated into the leaf inter-cellular spaces using either a blunt-ended syringe or by means of a vacuum infiltrator, applying a vacuum of 100 kPa. For optimization of the expression, 3 leaf discs were obtained from each plant, clipped with the lid of a micro-centrifuge tube on 3, 5 and 7 days post infiltration (dpi) and homogenized in 3 volumes of PI buffer (phosphate buffered saline (PBS), pH 7.4 containing 1× Complete protease inhibitor cocktail (Roche®, Basel, Switzerland)) using a micro-pestle. The homogenate was incubated on ice for 30 min and then clarified by centrifugation at 13 000 rpm for 15 min in a benchtop microfuge. For large scale expression, leaf tissue was harvested 7 dpi, as this time span was shown to be optimal for expression of all four capsid proteins. Harvested leaves were immediately homogenized in 3 volumes PI buffer using a Moulinex™ juice extractor. The homogenized leaves were re-incubated with the extracted juice and incubated at 4 OC for 1 h with gentle shaking. Crude plant extracts were filtered through four layers of Miracloth™ (Merck™, Darmstadt, Germany) and the filtrate was clarified by centrifugation at 13 000 rpm for 15 min at 4° C.

AHSV-5 Capsid Proteins Transiently Expressed in N. benthamiana Leaves Self-Assemble Into VLPs

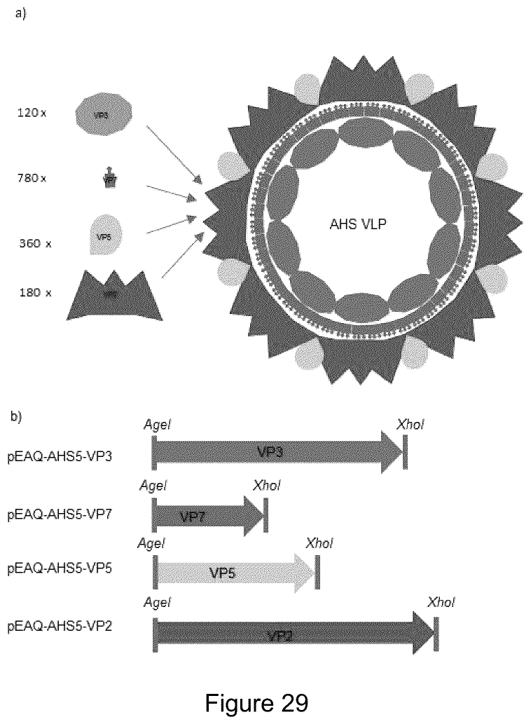

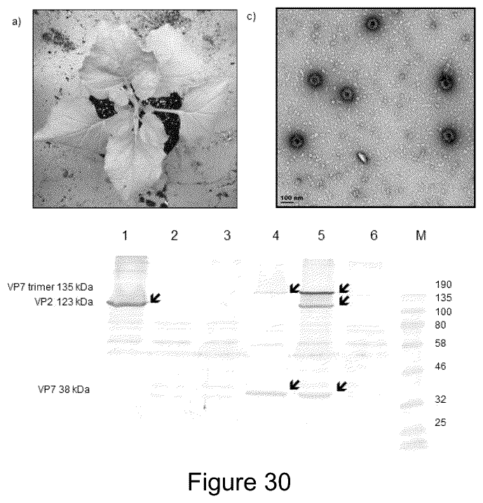

A consensus sequence of each gene was obtained by aligning all the known sequences listed in GenBank and these were codon-optimized for Nicotiana spp. translation and synthesized with flanking AgeI and XhoI restriction enzyme sites by GenScript Biotech Corporation™, China. The genes were cloned into the multiple cloning site of the pEAQ-HT vector (Sainsbury et al. 2009, obtained from G. Lomonossoff, John Innes Centre, UK) to yield four different constructs, PEAQ-AHS5-VP2, PEAQ-AHS5-VP3, PEAQ-AHS5-VP5 and PEAQ-AHS5-VP7 (FIG. 29b) Transient expression of the AHSV proteins in N. benthamiana was tested by small-scale syringe infiltration of 5 leaves with Agrobacterium strains carrying individual constructs, or co-infiltration of the same plant with all four recombinant-carrying strains. All infiltrated leaf tissue exhibited chlorosis, but little, if any, necrosis was observed (FIG. 30a). Agrobacterium suspensions carrying recombinants in two different VP2:VP3:VP5:VP7 ratios were tested, namely 1:1:1:1 and 1:1:2:1 and 3 leaf discs were extracted on days 5 and 7 post infiltration, to determine the optimal expression conditions. Western blots of crude leaf extracts infiltrated with Agrobacterium-carrying recombinants in a 1:1:1:1 ratio at an OD₆₀₀ of 0.5 each and prepared 7 days after infiltration were shown to yield optimal protein expression. Expression of VP2 (123 kD) and VP7 (37 kD), as well as the VP7 trimer (135 kD) was demonstrated, the proteins being visualized as distinct bands of the correct expected molecular weight. Bands corresponding to VP3 (103 kD) and VP5 (57 kD) were not observed (FIG. 30b). However, fully formed AHSV-5 VLPs were imaged by TEM analysis of these crude extracts, indicating that all four capsid proteins were expressed and indeed had self-assembled into complete particles (FIG. 30c). As such, this is the first known report of AHSV VLPs being produced in plants.

### Purification and Western Blot Analysis

AHSV-5 VLPs were purified by iodixanol density gradient ultracentrifugation. Iodixanol (Optiprep™, Sigma-Aldrich®, Missouri, USA) solutions (20-60%), prepared in PBS, were used to create a 12 ml step gradient (2-3 mL of each gradient in 10% incrementing steps) under 27 ml clarified plant extract and centrifuged at 32 000 rpm for 2 h at 4° C. in an SW 32 Ti rotor (Beckman®, California, USA). Fractions of 1 ml were collected from the bottom of the tube and 30 μl from fractions representing the 30-40% region of the gradient were electrophoresed on a 10% SDS-polyacrylamide gel, followed by Coomassie blue staining. Particle quantification was achieved by visual comparison of the four capsid protein bands to known amounts of bovine serum albumin (BSA) run in separate lanes on the same SDS-PAGE gel. To further purify and concentrate VLP samples for use in animal studies, VLP-containing fractions were diluted with PBS to 20% iodixanol and subjected to a second round of ultracentrifugation per the same protocol described above. Both crude plant extracts and gradient-purified VLPs were analyzed by western blot: heat-denatured samples were separated on 10% polyacrylamide gels and then transferred onto HyBond™ C Extra nitrocellulose membranes (AEC-Amersham™, Gauteng, South Africa) using a Trans-blot™ SD semi-dry transfer cell (Bio-Rad™, California, USA). Membranes were first probed with a 1:1000 dilution of AHSV-5 specific horse serum (received from Deltamune™, Pretoria, South Africa), washed four times with PBS containing 0.05% Tween™ 20 (Sigma-Aldrich®, Missouri, USA) (PBS-T) and then probed with 1:5000 dilution of anti-horse alkaline phosphatase-conjugated secondary antibody (Sigma-Aldrich®, Missouri, USA). After washing again, proteins were detected with 5-bromo-4-chloro-3-indoxyl-phosphate (BCIP) and nitroblue tetrazolium (NBT) phosphatase substrate (BCIP/NBT 1-component, KPL, SeraCare™, Massachusetts, USA).

### Density Gradient Ultracentrifugation of Plant-Produced AHSV-5 VLPs

To produce an AHS VLP preparation of sufficient purity and concentration for immunization of guinea pigs, several modifications were made. Firstly, the process was scaled up to infiltrate 24 plants with the recombinant constructs at the optimal OD₆₀₀ of 0.5 each and optimal ratio of 1:1:1:1. Secondly, AHSV VP7 is known to form trimers which aggregate into crystalline structures in the cytoplasm of infected cells and there is evidence to suggest that these crystals impede VLP formation by sequestering available soluble VP7 trimers and preventing them from incorporating into the core particle. Therefore, a mutated version of the VP7 gene containing 7 amino acid substitutions near the 3′ end was also synthesized (SEQ ID NO:77) and cloned into pEAQ-HT to yield pEAQ-AHS5-VP7mu. The protein encoded by the mutated version of the VP7 gene has the sequence set forth in SEQ ID NO:78. Co-infiltration with Agrobacterium strains carrying the VP2, VP3 and VP5 recombinants together with this construct as opposed to the wild-type VP7 construct, yielded an increased concentration of VLPs. Therefore, the mutated VP7 construct was used in all further experiments. Thirdly, a vacuum infiltrator was used to introduce the Agrobacterium suspension into the leaf intercellular spaces as this was much less labour intensive than syringe infiltration and resulted in more uniform infiltration of plant leaves.

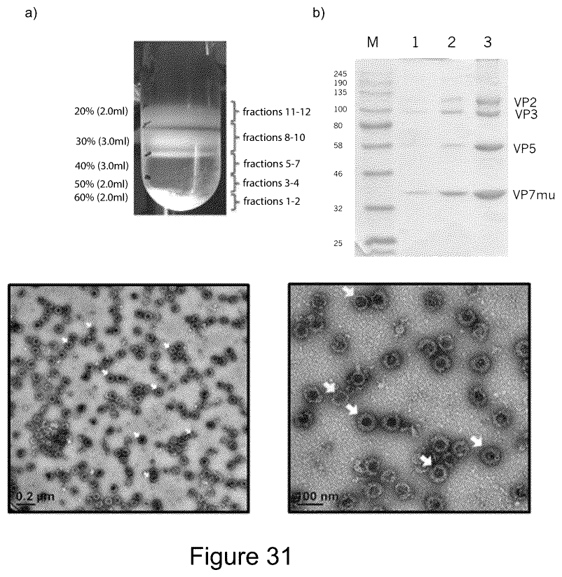

Lastly, clarified leaf extracts were purified by iodixanol density gradient ultracentrifugation. Green leaf impurities settled in the upper 30% region of the gradient, while a single iridescent band was observed at a higher density, near the 30-40% interface (FIG. 31a). Fractions were collected from the bottom of the tube and four distinct bands corresponding to the correct molecular weight sizes of the AHSV capsid proteins were observed following separation of fractions 6-8 by SDS-PAGE and Coomassie blue staining (FIG. 31b). Gel densitometry was used to estimate the VLP concentration. The co-sedimentation of all four proteins was highly suggestive of the presence of VLPs and this was confirmed by TEM analysis (FIG. 31c). An estimated 40-50% of the viral structures were seen to be complete AHSV VLPs or contained at least a partial VP2 outer layer, though some particles appear to have been slightly damaged during the purification process. Assembly intermediates representing core-like particles (CLPs) or CLPs in the process of acquiring the two outer coat proteins, were also observed. This purification has been repeated several times and typically, 70 g infiltrated leaf material yields ±0.4 mg highly purified VLPs which equates to ±5.7 mg VLPs/kg leaf biomass.

### Transmission Electron Microscopy

Glow-discharged copper grids (mesh size 200) were floated on 20 μl crude plant extract or 20 μl density gradient fractions for 3 min and then washed successively by floating on 5 drops of sterile water. Particles were negatively stained for 30 sec with 2% uranyl acetate and then imaged using a Technai™ G2 transmission electron microscope (TEM).

### Immunization of Guinea Pigs

Approval for the immunization experiments was obtained from the Faculty of Health Sciences Animal Ethics Committee, University of Cape Town (FHS AEC ref No.: 016/019). Prior to the study, 100 μl of blood was drawn from each of 8 female guinea pigs (Hartley strain). Guinea pigs (n=4) were injected subcutaneously with purified AHSV-5 VLPs or 30% iodixanol in PBS, both formulated in 5% Montanide™ PET Gel A adjuvant (Seppic™, Paris, France). Animals were boosted on day 13 and on day 41, they were euthanized by anaesthesia with ketamine/xylazine and exsanguinated. Serum was tested for antibodies by indirect enzyme-linked immunosorbent assay (ELISA) and western blot. Briefly, 96-well Maxisorp™ microtiter plates (Thermo Fisher Scientific®, Massachusetts, USA) were coated overnight at 4° C. with 60 ng/well of AHSV-5 VLPs originally used for the inoculations. Plates were washed four times with PBS-T and blocked with 5% fat-free milk powder diluted in PBS-T for 1 h at 37° C. Guinea pig antisera were serially diluted in PBS-T/5% milk, added to the plates and allowed to incubate for 1 h at 37° C. Plates were washed four times, and an alkaline phosphatase-conjugated goat anti-guinea pig IgG (Sigma-Aldrich®, Missouri, USA) was diluted (1:5000) in blocking buffer and added to plates. Plates were again incubated for 1 h at 37° C. and washed four times. After addition of 100 μl p-Nitrophenyl phosphate substrate (SIGMAFAST™, Sigma-Aldrich®, Missouri, USA), the plates were incubated in the dark for 30 min to allow a colorimetric reaction to develop. Optical densities at a wavelength of 450 nm were read by a Bio-Tek™ Powerwave XS spectrophotometer. For western blot analysis, guinea pig antisera were used at a dilution of 1:10 000 as per the protocol described above.

### Neutralization Assays

The serum neutralizing antibody titres of individual guinea pig sera were assayed against three different AHSV serotypes, namely serotypes 4, 5 and 8 using a serum neutralization test (SNT).

### Plant-Produced AHSV-5 VLPs Induce a Strong Immunogenic Response in Guinea Pigs

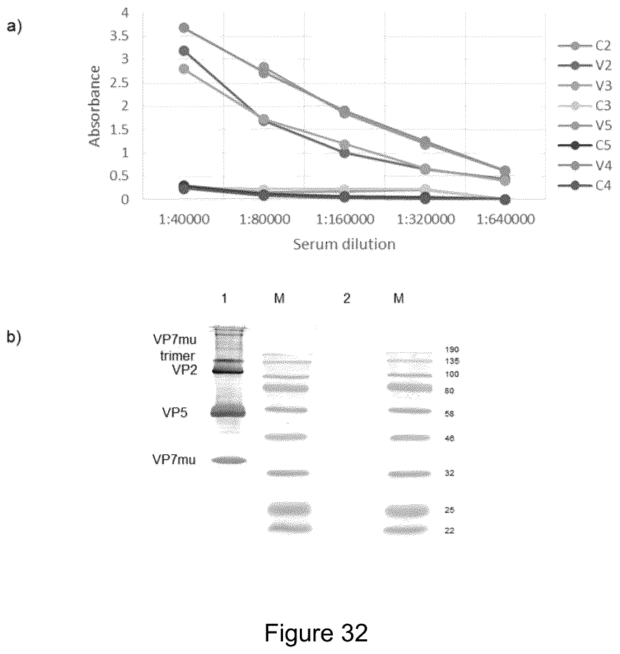

Guinea pigs were used as a small animal model to test the ability of the plant-produced AHSV 5 VLPs to induce an immune response. On day 0, four guinea pigs (V2-V5) were each vaccinated with 16.5 μg AHSV VLPs, while four control animals (C2-C5) were immunized with PBS. Prior to the boost inoculation, a further purification yielded sufficient AHS VLPs to increase the amount of the next inoculum. Animals were thus boosted on day 13 with 50 μg VLPs or PBS and sera from all animals was collected on day 41. Sera from guinea pigs immunized with VLPs tested positive for AHSV 5 antibodies in indirect ELISA, 1:40 000 being the lowest dilution at which an absorbance value could be read. Sera from guinea pigs vaccinated with PBS, tested negatively (FIG. 32a). Final and pre-bleed sera (1:10 000) from a representative VLP-vaccinated guinea pig (V3) were used to probe a western blot of VLPs used in the initial inoculations. Strong signals for VP2, VP5 and VP7 (both monomer and trimer) were detected by the final bleed serum but not by the pre-bleed serum from the vaccinated guinea pig (FIG. 32b).

To test the ability of the sera to neutralize live virus, serum samples from all guinea pigs were sent to the Equine Research Centre at Onderstepoort, University of Pretoria for serum neutralization tests. Sera were assayed against AHSV-5 and AHSV-8 as serological cross-protection has been shown in vitro between serotypes 5 and 8, and AHSV-4 for which no cross protection has been shown. All vaccinated guinea pig sera showed a high level of neutralization capability against AHSV-5 and neutralized AHSV-8 to a lesser extent, but to a similar degree compared to the AHS positive control (Table 11). The sera did not neutralize AHSV-4 and control guinea pig sera did not neutralize any of the AHSV serotypes. These results indicate that plant-produced AHSV-5 VLPs stimulate a highly protective immune response in guinea pigs.

TABLE 11
Virus neutralizing antibody titers of serum samples from vaccinated (V) and
control (C) guinea pigs. The guinea pig sera were assayed for neutralization capability against
AHSV-5, AHSV-4 and AHSV-8, as serological cross-protection has been shown in vitro
between serotypes 5 and 8, but not between serotypes 5 and 4. Horse serum from animals
vaccinated with the AHSV live-attenuated vaccine produced by Onderstepoort Biological
Products (OBP) was used as a positive control.

| Group | Guinea Pig | AHSV-4 | AHSV-5 | AHSV-8 |
|---|---|---|---|---|
| Vaccine | V2 | Negative | 1:5120 | 1:160 |
| | V3 | Negative | 1:640 | 1:80 |
| | V4 | Negative | 1:1280 | 1:56 |
| | V5 | Negative | 1:2560 | 1:80 |
| Control | C2 | Negative | Negative | Negative |
| | C3 | Negative | Negative | Negative |
| | C4 | Negative | Negative | Negative |
| | C5 | Negative | Negative | Negative |
| OBP vaccine | — | 1:112 | 1:112 | 1:112 |

### Example 10

### Production of BT VLPs Harnessing Various Agrobacterium Strains

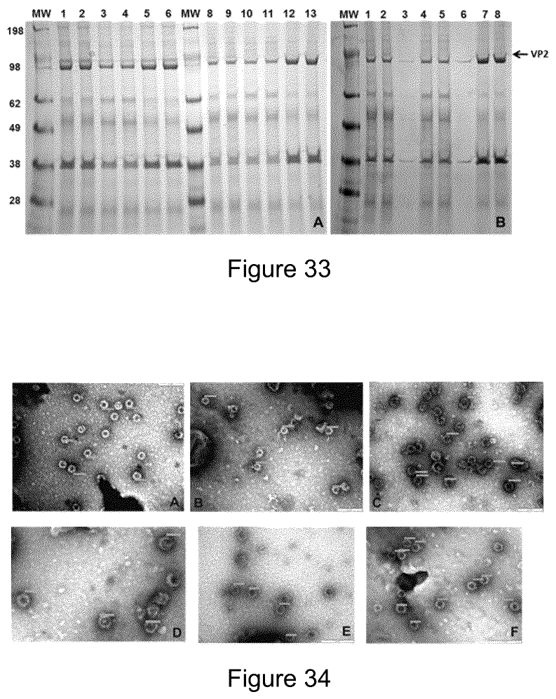

LBA4404, AGL-1 and GV3101 pMP90 Agrobacterium strains were compared as vehicle to deliver the expression vector pEAQ-HT, harbouring the selected genes, to the plant cells. BTV-8 (VP3, VP7, VP5 and VP2) and BTV-3 (VP5 and VP2) were individually electroporated into these Agrobacterium strains. The goal was to determine the Agrobacterium strain most suitable and resulting in the highest number of intact double chimaeric BTV-3 (BTV-8 VP3 and VP7 core combined with BTV-3 VP2 and VP5 outer capsids) and homogenous BTV-8 VLPs (BTV-8 VP3, VP5, VP2 and VP7) for commercial production. Assembly of BTV serotypes 3 and 8 VLPs were created using stocks from the LBA4404 seed cell bank, or the recently prepared Agrobacterium AGL-1 and GV3101 pMP90 collection. N. benthamiana dXT/FT plants were infiltrated with the relevant Agrobacterium and construct combinations. The Agrobacterium strains harboring PEAQ-HT constructs encoding for the four capsid proteins individually for BTV serotypes 3 and 8 were successfully infiltrated into N. benthamiana leaves. Production of VLPs in plant leaf tissue was determined by mixing the four constructs encoding the four individual capsid proteins VP3:VP7:VP5:VP2 at a ratio of 1:1:1:1 (OD₆₀₀=2). Leaf tissue was harvested seven days after infiltration, extracted and lodixanol density gradient purified as described above. The lodixanol purified BT VLPs proteins were quantified using a sensitive colorimetric protein assay, the Micro BCA™ Protein Assay Kit (ThermoScientific™) using Bovine Gamma Globulin (Bio-Rad™) protein standards. Eight micrograms of protein were loaded in each lane (FIG. 33).

The lodixanol samples were stained as follows: grids were floated on the undiluted protein sample for 5 minutes were washed five times in 5 μl distilled water, drained via blotting on filter paper each time before staining. Subsequently the grids were floated on 2% uranyl acetate (30 seconds, drained and stained for another 10seconds) and drained as described above. The air dried grid was imaged in a CM10 Transmission electron microscope (Philips™) at the University of Pretoria (UP) Onderstepoort, Laboratory for Microscopy and Microanalysis (FIG. 34). Mass spectrometry was conducted as described before.

In this Example, Agrobacterium strains LBA4404, GV3101 pMP90 and AGL-1 were compared to mediate homogenous BTV-8 and double chimaeric BTV-3 in N. benthamiana facilitating mammalian glycosylation (dXT/FT, Strasser et al., 2008) by exclusively subjecting VP2 to mass spectrometry. Assembly of homogenous BTV-8 and double chimaeric BTV-3 as visualized by TEM images is comparable when mediated by the three independent Agrobacterium strains. Mass spectrometry analysis of homogenous BTV-8 VLPs (duplicate technical replicates) indicated that either LBA4404 (42-46 peptides) or GV3101 pMP90 (36-41 peptides) or AGL-1 (49-52 peptides) are suitable to mediate abundant VLP assembly with strain AGL-1 slightly superior. Mass spectrometry analysis of double chimaeric BTV-3 (triple technical replicates) however indicated that LBA4404 (32-39 peptides) is superior to both GV3101 pMP90 (7 peptides in only one sample) and AGL-1 (14-17 peptides).

TABLE 12
Mass spectrometry results of the production of BT VLPs harnessing various
Agrobacterium strains

| | Duplicates | | | | | |
|---|---|---|---|---|---|---|
| | or | | | | | |
| | triplicates | | | | | |
| | of VP2 | Viral | | | 95% | |
| Sample # | detected | protein | Note | Peptides | coverage | BTV VP2 serotypes |
| BTV-8 LBA4404 | 1 | BTV-8 VP2 | 111 kDa | 46 | 48.0% | BTV8_VP2_AGJ83482_1 |
| Homogenous | 2 | BTV-8 VP2 | 111 kDa | 42 | 40.0% | BTV8_VP2_AGJ83482_1 |
| BTV-8 GV3101 | 1 | BTV-8 VP2 | 111 kDa | 41 | 43.0% | BTV8_VP2_AGJ83482_1 |
| pMP90 | | | | | | |
| Homogenous | 2 | BTV-8 VP2 | 111 kDa | 36 | 37.0% | BTV8_VP2_AGJ83482_1 |
| BTV-8 AGL-1 | 1 | BTV-8 VP2 | 111 kDa | 49 | 51.0% | BTV8_VP2_AGJ83482_1 |
| Homogenous | 2 | BTV-8 VP2 | 111 kDa | 52 | 47.0% | BTV8_VP2_AGJ83482_1 |
| BTV-3 LBA4404 | 1 | BTV-3 VP2 | 111 kDa | 32 | 0.35% | BTV3_VP2_CAE51090_1 |
| Double chimaeric | 2 | BTV-3 VP2 | 111 kDa | 39 | 0.4% | BTV3_VP2_CAE51090_1 |
| | 3 | BTV-3 VP2 | 111 kDa | 26 | 0.28% | BTV3_VP2_CAE51090_1 |
| BTV-3 GV3101 | 1 | BTV-3 VP2 | 111 kDa | 7 | 0.06% | BTV3_VP2_CAE51090_1 |
| pMP90 | | | | | | |
| Double chimaeric | 2 | BTV-3 VP2 | 111 kDa | Not detected | | BTV3_VP2_CAE51090_1 |
| | 3 | BTV-3 VP2 | 111 kDa | Not detected | | BTV3_VP2_CAE51090_1 |
| BTV-3 AGL-1 | 1 | BTV-3 VP2 | 111 kDa | 17 | 0.21% | BTV3_VP2_CAE51090_1 |
| Double chimaeric | 2 | BTV-3 VP2 | 111 kDa | 14 | 0.14% | BTV3_VP2_CAE51090_1 |
| | 3 | BTV-3 VP2 | 111 kDa | Not detected | | BTV3_VP2_CAE51090_1 |

### REFERENCES

COETZER, J.A.W. & GUTHRIE, A.J. (2004) African horse sickness. In: Infectious Diseases of Livestock 2nd ed. Oxford University Press Southern Africa, Cape Town, 1231-1246.
DEMAULA, C. D., BONNEAU, K. R. & MACLACHLAN, N. J. 2000. Changes in the outer capsid proteins of bluetongue virus serotype ten that abrogate neutralization by monoclonal antibodies. Virus Res, 67, 59-66.
HUISMANS, H. & ERASMUS, B. 1981. Identification of the serotype-specific and group-specific antigens of bluetongue virus. Onderstepoort J Vet Res, 48, 51-58.
MERTENS, P. P., DIPROSE, J., MAAN, S., SINGH, K. P., ATTOUI, H. & SAMUEL, A. R. 2004. Bluetongue virus replication, molecular and structural biology. Vet Ital, 40, 426-37.
PEARSON, L.D. & ROY, P. 1993. Genetically engineered multi-component virus-like particles as veterinary vaccines. Immunology and Cell Biology 71:381-389.
SAINSBURY, F., THUENEMANN, E. C. & LOMONOSSOFF, G. P. 2009. PEAQ: versatile expression vectors for easy and quick transient expression of heterologous proteins in plants. Plant Biotechnol., 7, 682-693.
SHEVCHENKO, A., TOMAS, H., HAVLIŠ, J., OLSEN V. J. & MANN, M. (2007) In-gel digestion for mass spectrometric characterization of proteins and proteomes. Nature Protocols. 1, 2856-2860.
STRASSER R, STADLMANN J, SCHÄHS M, STIEGLER G, QUENDLER H, MACH L, GLÖSSL J, WETERINGS K, PABST M & STEINKELLER H, 2008. Generation of glycol-engineered Nicotiana benthamiana for the production of monoclonal antibodies with a homogenous human N-like N-glycan structure. Plant Biotechnology Journal 6:392-402.
THUENEMANN, E. C., MEYERS, A. E., VERWEY, J., RYBICKI, E. P. & LOMONOSSOFF, G. P. 2013. A method for rapid production of heteromultimeric protein complexes in plants: assembly of protective bluetongue virus-like particles. Plant Biotechnology Journal, n/a-n/a.

That which is claimed:

1. A method of producing a chimaeric Blue Tongue Virus (BTV) VLP in a plant cell, the method comprising:
(i) providing codon-optimised nucleotide sequences encoding BTV VP2, VP3, VP5 and VP7 structural proteins, wherein at least one of the VP2, VP3, VP5 and VP7 structural proteins is selected from a first BTV serotype and at least one of the VP2, VP3, VP5 and VP7 structural proteins is selected from a second BTV serotype;
(ii) cloning the codon-optimised nucleotide sequences into at least one expression vector adapted to express the structural proteins in a plant cell;
(iii) transforming or infiltrating the plant cell with the at least one expression vector of step (ii);
(iv) co-expressing the VP2, VP3, VP5 and VP7 structural proteins in the plant cell, such that the expressed structural proteins assemble to form the chimaeric BTV VLP; and
(v) recovering the chimaeric BTV VLP from the plant cell.

2. The method of claim 1, wherein in step (i) at least one of the VP2, VP3, VP5 and VP7 structural proteins is selected from a third BTV serotype.

3. The method of claim 2, wherein at least one of the VP2, VP3, VP5 and VP7 structural proteins is selected from a fourth BTV serotype.

4. The method of claim 1, wherein the structural proteins are transiently expressed in the plant cell.

5. The method of claim 1, wherein the at least one expression vector includes a promoter and/or other regulatory sequences, operably linked to each nucleotide sequence encoding each structural protein.

6. The method of claim 1, wherein in step (iii) the at least one expression vector is transformed into the plant cell in a ratio of 1:1:1:1 or a ratio of 1:1:2:1 or a ratio of 2:1:2:1 of the nucleotide sequences encoding VP2:VP3:VP5:VP7.

7. The method of claim 1, wherein the plant cell is a Nicotiana benthamiana cell.

8. The method of claim 1, wherein the plant cell is a mutant N. benthamiana dXT/FT tobacco cell, which facilitates mammalian-like or human-like glycosylation of polypeptides.

9. The method of claim 1, wherein the expression of the BTV VP2, VP3, VP5 and VP7 structural proteins in the plant cell is mediated by Agrobacterium AGL-1, LBA4404 or GV3101 pMP90.


SEQUENCE LISTING (84 sequences)

SEQ ID NO: 1 - protein, 901 aa, Bluetongue virus
MAAQNEQRPERIKTTPYLEGDVLSSDSGPLLSVFALQEIMQKVRQVQADYMTATREVDFTVPDVQKILDDIKALAAEQVYKIVKVPSISFRHIVMQSRDRVLRVDTYYEEMSQVGDVITEDEPEKFYSTIIKKVRFIRGKGSFILHDIPTRDHRGMEVAEPEVLGVEFKNVLPVLTAEHRAMIQNALDGSIIENGNVATRDVDVFIGACSEPIYRIYNRLQGYIEAVQLQELRNSIGWLERLGQRKRITYSQEVLTDFRRQDTIWVLALQLPVNPQVVWDVPRSSIANLIMNIATCLPTGEYIAPNPRISSITLTQRITTTGPFAILTGSTPTAQQLNDVRKIYLALMFPGQIILDLKIDPGERMDPAVRMVAGVVGHLLFTAGGRFTNLTQNMARQLDIALNDYLLYMYNTRVQVNYGPTGEPLDFQIGRNQYDCNVFRADFATGTGYNGWATIDVEYRDPAPYVHAQRYIRYCGIDSRELINPTTYGIGMTYHCYNEMLRMLVAAGKDSEAAYFRSMLPFHMVRFARINQIINEDLHSVFSLPDDMFNALLPDLIAGAHQNADPVVLDVSWISLWFAFNRSFEPTHRNEMLEIAPLIESVYASELSVMKVDMRHLSLMQRRFPDVLIQARPSHFWKAVLNDSPEAVKAVMNLSHSHNFINIRDMMRWVLLPSLQPSLKLALEEEAWAAANDFEDLMLTDQVYMHRDMLPEPRLDDIERFRQEGFYYTNMLEAPPEIDRVVQYTYEIARLQANMGQFRAALRRIMDDDDWVRFGGVLRTVRVKFFDARPPDDILQGLPFSYDTNEKGGLSYATIKYATETTIFYLIYNVEFSNTPDSLVLINPTYTMTKVFINKRIVERVRVGQILAVLNRRFVAYKGKMRIMDITQSLKMGTKLAAPTV

SEQ ID NO: 2 - protein, 901 aa, Bluetongue virus
MAAQNEQRPERIKTTPYLEGDVLSSDSGPLLSVFALQEIMQKVRQVQADYMTATREVDFTVPDVQKILDDIKALAAEQVYKIVKVPSISFRHIVMQSRDRVLRVDTYYEEMSQVGDVITEDEPEKFYSTIIKKVRFIRGKGSFILHDIPTRDHRGMEVAEPEVLGVEFKNVLPVLTAEHRAMIQNALDGSIIENGNVATRDVDVFIGACSEPIYRIYNRLQGYIEAVQLQELRNSIGWLERLGQRKRITYSQEVLTDFRRQDTIWVLALQLPVNPQVVWDVPRSSIANLIMNIATCLPTGEYIAPNPRISSITLTQRITTTGPFAILTGSTPTAQQLNDVRKIYLALMFPGQIILDLKIDPGERMDPAVRMVAGVVGHLLFTAGGRFTNLTQNMARQLDIALNDYLLYMYNTRVQVNYGPTGEPLDFQIGRNQYDCNVFRADFATGTGYNGWATIDVEYRDPAPYVHAQRYIRYCGIDSRELINPTTYGIGMTYHCYNEMLRMLVAAGKDSEAAYFRSMLPFHMVRFARINQIINEDLHSVFSLPDDVFNALLPDLIAGAHQNADPVVLDVSWISLWFAFNRSFEPTHRNEMLEIAPLIESVYASELSVMKVDMRHLSLMQRRFPDVLIQARPSHFWKAVLNDSPEAVKAVMNLSHSHNFINIRDMMRWVLLPSLQPSLKLALEEEAWAAANDFEDLMLTDQVYMHRDMLPEPRLDDIERFRQEGFYYTNMLEAPPEIDRVVQYTYEIARLQANMGQFRAALRRIMDDDDWVRFGGVLRTVRVKFFDARPPDDILQGLPFSYDTNEKGGLSYATIKYATETTIFYLIYNVEFSNTPDSLVLINPTYTMTKVFINKRIVERVRVGQILAVLNRRFVAYKGKMRIMDITQSLKMGTKLAAPTV

SEQ ID NO: 3 - protein, 526 aa, Bluetongue virus
MGKIIKSLSRFGKKVGNALTSNTAKKIYSTIGKAAERFAESEIGSAAIDGLVQGSVHSLMTGESYGESVKQAVLLNVMGSGEELPDPLSPGERGMQTKIRELEDEQRNELIRLKYNDKIKQKFGKELEEVYEFMNGVAKQEEDEEKHYDVLKKAVNSYDKILTEEEKQMRILATALQKEVKERTGTEAVMVKEYRNKIDALKEAIEVERDGMQEEAIQEIAGMTADVLEAASEEVPLIGAGMATAVATGRAIEGAYKLKKVINALSGIDLTHLRTPKIEPTIVSTVLDHKFKDIPDEMLAVSVLSKNRAIEENHKEIIHLKNEILPRFKKAMDEEKEICGIEDKKIHPKVMMKFKIPRTQQPQIHIYSAPWDSDDVFFFHCISHHHANESFFIGFDLGIDLVHYEDLTAHWHALGAAQAAVGRSLNEVYKEFLNLAINNTYSSQMHARRMIRSKTVHPIYLGSLHYDISFSTLRSNAQRIVYDEELQMHILRGPLHFQRRAILGAIKHGVKILGTEVDIPLFLRNA

SEQ ID NO: 4 - protein, 526 aa, Bluetongue virus
MGKIIKSLSRFGKKVGNALTSNTAKKIYSTIGKAAERFAESEIGSAAIDGLVQGSVHSLMTGESYGESVKQAVLLNVMGSGEELPDPLSPGERGMQTKIRELEDEQRSELIRLKYNDKIKQKFGKELEEVYEFMNGVAKQEEDEEKHYDVLKKAVNSYDKILTEEEKQMRILATALQKEVKERTGTEAVMVKEYRNKIDALKEAIEVERDGMQEEAIQEIAGMTADVLEAASEEVPLIGAGMATAVATGRAIEGAYKLKKVINALSGIDLTHLRTPKIEPTIVSTVLDHKFKDIPDEMLAVSVLSKNRAIEENHKEIIHLKNEILPRFKKAMDEEKEICGIEDKKIHPKVMMKFKIPRTQQPQIHIYSAPWDSDDVFFFHCISHHHANESFFIGFDLGIDLVHYEDLTAHWHALGAAQAAVGRSLNEVYKEFLNLAINNTYSSQMHARRMIRSKTVHPIYLGSLHYDISFSTLRSNAQRIVYDEELQMHILRGPLHFQRRAILGAIKHGVKILGTEVDIPLFLRNA

SEQ ID NO: 5 - protein, 349 aa, Bluetongue virus
MDTIAARALTVMRACATLQEARIVLEANVMEILGIAINRYNGLTLRGVTMRPTSLAQRNEMFFMCLDMMLSAAGINVGPISPDYTQHMATIGVLATPEIPFTTEAANEIARVTGETSTWGPARQPYGFFLETEETFQPGRWFMRAAQAVTAVVCGPDMIQVSLNAGARGDVQQIFQGRNDPMMIYLVWRRIENFAMAQGNSQQTQAGVTVSVGGVDMRAGRIIAWDGQAALHVHNPTQQNAMVQIQVVFYISMDKTLNQYPALTAEIFNVYSFRDHTWHGLRTAILNRTTLPNMLPPIFPPNDRDSILTLLLLSTLADVYTVLRPEFAIHGVNPMPGPLTRAIARAAYV

SEQ ID NO: 6 - protein, 349 aa, Bluetongue virus; identical to SEQ ID NO: 5

SEQ ID NO: 7 - protein, 961 aa, Bluetongue virus
MEELAIPIYTNVFPAELLDGYDYIIDVSSRVEEEGDEPVKRHDVTEIPRNSMFDIKDEHIRDAIIYKPVNNDGYVLPRVLDITLKAFDDRKRVVLNDGHSEFHTKTNWVQWMIDDAMDVQPLKVDIAHTRSRISHALFNCTVRLHSKKADTASYHVEPVEIESWGCNHTWLSRIHHLVNVELFHCSQEAAYTLKPTYKIISNAERASTSDSFNGTMIELGRNHQIQMGDQGYQKLKEGLVQVRIEGKTPLVIQEEITALNKIREQWIARNFDQREIKVLDLCRLLSTIGRKMCNTEEEPKNEADLSVKFQMELDEIFRPGNNERTNIMGGGVHRKNEDRFYVLIMIAASDTNKGRIWWSNPYPCLRGALIAAEVQLGDVYNLLRNWFQWSVRPTYVPYDRNRESDKYIYSRINLFDSTLRPGDKIVHWEYKLLNEVREVSINKGNECDLFPEDEEFTTKFHEARYTEMKNQIIQSGWNQRDFKMHKILEDGANVLTIDFEKDAHIGTGSALSLPDYYNKWIIAPMFNAKLRITEVVIGTAHTDDPAVGRSAKAFTHDPFDLQRYCLARYYDVRPGMMGRALSKQQNMSSMTDKLSKQEDYAGIVSRRLEYKERENRCLTETAQYVFEKTCLYVLELLSRHTMPSEDSEVTFEHPTIDPSVDIETWKIIDVSQLIIFVFDYLFENRKIVRDTTEARWTLFKIRSEVGRARIDAIEMTFPRFGRMLRNASQAKINQDIACLNFLPLLFIIGDNISYAHRQWSIPVLLYAHDIRIIPLEVGAYNNRFGLTSYLEYMAFFPSYATRVAKIDESIKECAIAMAEFYMNTDIHSGSVMSNVITTKRLLYETYLASLCGGYSDGLLWYLPITHPSKCLVAFEVADDVVPLSVRRERILSRFPLSSRHVKGIALISVDRNQKVSVQTEGIVTHRLCKKNLLKYVCDVILFKFSGHVFGNDEMLTKLLNV

SEQ ID NO: 8 - protein, 962 aa, Bluetongue virus
MDELGIPIYGRNYPEHLLKGYEFLINTGVRYPSQGGRHDVSKIPEMFAYDIKDEGIREALKYMPTRNDGVVLPRIVDISLKGYDIRKSVIEANKSNSFHTDTNWVQWMIKDSMDQQPLKISIDEEHSRVVHSLFNCQVKIDAKKADTLSYHLEAIEDAEKACLHTRGQLCNHLARMDLLHAAQEIAYAIKPTYQLIVHSERASTSDNFELGRQDVITLRRGHRIQMGDEAYTKLMERLVRLTVQGNVPRKIQSEIEQLEAIRTRWATGRYDPAHINSQDLCRILSRIGRIMLDQEAEPVDEDSLSLRFQRALDEKFRLNDSERNKIFEPKSHRKDEDRFYVLLAIAASDTYNSRIWWSNPYPCLRGTLIAAETKLGDVYFTLRSWYDWSVRSSYVPRERERETEKYIFSKINLFDYEAGPSSKVIHWEYQLYKRERVVTLERGNPCDLYPDEDDEVIITKFDDAKYSEMVGEIIDGGWNDEEFKMYKLLQEKGNVLTIDFEKDTKLNSTSEVVLPDYYGKWIVAPMFNSKMRIIETEIATNRSDDPMIKRTLKPMTDDPVELQRYTLARYYDIRPGLMGRSLNRTQTQSTFDAKVSELSDYEKVVSRFGVIKKPTRPCVTLTGRYILEKYSLLLIDILKYHTEVEENPQEEFTHPRIDLQFKFNGNTLSDLNQTVVFIVDYLHEKRNYVRSVYEARYIISRIRSSTGAARMSILEFYFPTFARLISNAREPTYVKDLMALNFLPLLFIVGDNMIYKHRQWSIPLLLYTDRVKVIPLEVGSSNNRQGLVSYLEYMFFFPSLADRTSKVDESMIKVSKEVVNYYMKTTISEGGVNLNVVSTKSLLYDIYLSSVCGGVSDGVVWYLPITHPYKCVVAIEVCDDRVPARLRCDRLRLRFPLSAQHLKGIVVIQINEEGGFDVYTEGIVTHRVCKKSLLKHVCDIVLLKFHGHVFGNDEMLTKLLNV

SEQ ID NO: 9 - protein, 959 aa, Bluetongue virus
MEELVIPVISRQFDKKLVGRYEYVIELAEPEQDEWTNHDVTQIPGRRMFDVAQQGIREAIIYKPLDNDGEVLPRILDMSIACYDMRKTMMKKEGVDFVSNTRWLEWMIQDSMDVQPLRVQMKEDHSTVQYDMFSAKVHIDSRKADTTSYHAIAVETKAERKCCHVRTEVWNSVVRNHLFNTAQESCYTFKQTYELIVNSERLSTEEEFRVGAPQFHTIQRNHRMQLGDNAYDKFLKGLVQLRVSGTTPAKIRDEVAALDVIRDNWIRGSFDRSHIKSLELCRLLSSIGRKMVNMEEEPKDEKDLSVKFQFRLDEKFSPNDPERNVIFTSKTHRTNEDRFYVLLVIAASDTNNGRVWWSNPYPCLRGALIAAECKLGDVYHTLRSKYEWGVRPTYKPKDLEREREKYVVGRVNLFDLEGEPATKVIHWEYELISPTYSVSNHKGNHCDLYPDDVEITTKFNEDRYREMIQSVIDDGWDQKNLKMYKILEEEGNPLLYDLEKDIKLDSQSQVVFPSYYNKWTHAPMFNARVKPCDIELAERKNDDPFVKRTLKPIKADCVDLLRYHMSHYYDLRPSLKGVSLSNKQTPSGIHQALVQDDLYSRLLRRRDVDLDYSSPCPIITNYFLLEKFHILILTIMEKHYWELDDSDDVYEFPKIDASAFEVDGTLYDISQTIVHMYDRFFEKRRVLRSIDESRWILHLIRISQGRERLEVIERFFPNYGKAMRQRDFKKVRDVMFLNFLPFFFLTGDNISYEHRQWSIPIILYADKLRILPIEVGAHYNRFGVTCILELLNFFPSYEKREEKLEEDIVLCADAIVNFYLQTTISNGGVQTSIVSTKALLYEMYLSSICGGYSEGVLWYLPITHPVKCLVALEVSDALVGADVRIDKIRRRFPLSAKHLKGIVQISVHPNRTFSVTTCGIVKHKVCKKTLLKHRCDVILLQTPGYVFGNDELLTKLLNI

SEQ ID NO: 10 - protein, 956 aa, Bluetongue virus
MEEFVIPVFSEREIPYALINQYPLAIQTDVRVVDVDGNHNLVKIPESDMIDVPKLDIVSALNYKPTRNDGIVVPRLLDITLKAYDDRKSVKNARGVDFMTDAKWMKWAIDDRMDIQPLKITLDEHYSVNHQLFNCIVKAKTANADTIYYDYFPLEDRAKKCNHTNLELLRSLTTIEAFHILQGAAYSLKSNYDLIANSERESLEESYPIGSEKWVHLTRRTKIGNSGLSYNRFISSMVQVVVRGKVPDIIRGEITQLNRIRTEWIGASYDRTRIRALELCNILSAIGRKMMDTHEEPKDEMDLSTRFQFKLDEKFNTSDSEHVNIFRTSGAATNEGRFYALIAIAATDTQKGRVWRTNPYPCLRGALIASECELGDVYYTLRHVYQWSLRPEYGQREQQLEDNKYVFGRVNLFDSDLAVGDQIIHWQYEITEPVKTTYDDGYICNPEEEDDELLCKIDDERYKEMMERLIEGGWDQERFKLHSILTEPNLLTIDFEKDAYLNSRSELVFPNYFDKWINSPMFNARLRITHGEIGSSKTIDPWNRRVVYGYVKTSIESLDYALGRYYDIRLQLFGDTLSQKQTQSAVFTYLSEQDDFPALTNYSKGEAVCPHAGGAVYTFRKVALSLIANYEKLSPEMHEGLEHQMYVHPSANTTYQKQVKDMKDFSQLICFIIDCIFEKRVQIRGVGEARRIIYLIQNSTGSQRQEVLKKTFPNFFMRIFKLREVKRICDLSVINFLPLLFLVQDNISYWHRQWSVPMILFDDAVRLIPVEVGAYANRFGLKSFYNFVRFHPGDSKKKQDADDMHKEYGVACFKYYMNTKISQGGVNVPVVTSKLDTLKIHLASLCLGLADSIVYTLPVAHPKKCIVLIVVGDDKLDPQVRSEQVLSKYYYSRRHICGIVAVSVGQEGQLQVYSSGIVRHRICEKSILKYKCKVVLVRMPGHVFGNDELMTKLLNV

SEQ ID NO: 11 - protein, 961 aa, Bluetongue virus
MEELAIPIYTNVFPAELLDGYDYIIDISSRVEEEGDEPVKRHDVTEIPRNSMFDIKDEHIRDAIIYKPVNNDGYVLPRVLDITLKAFDDRKRVILNDGHSEFHTKSNWVQWMIDDAMDVQPLKVDIAHTRSRISHALFNCTVRLHSKKADTASYHVEPVEIESRGCNHTWLGRIHHLVNVELFHCSQEAAYALKPTYKIISNAERASTSDSFNGTMIELGRNHQIQMGDQGYQKLKEGLVQVRIEGKTPLAIQEEITALNKIREQWIARNFDQREIKVLDLCRLLSTIGRKMCNTEEEPKNEADLSVKFQMELDEIFRPGNNERTNIMGGGVHRKNEDRFYVLIMIAASDTNKGRIWWSNPYPCLRGALIAAEVQLGDVYNLLRNWFQWSIRPTYVPYDRNRESDKYIYSRINLFDSTLKPGDKIVHWEYELLNEVREVSISKGNECDLFPEDEEFTTKFHEARYTEMKNQIIQSGWNQRDFKMHKILEDGANVLTIDFEKDAHIGTGSALSLPDYYNKWIIAPMFNAKLRITEVVIGTAHTDDPAVGRSAKAFTHDPFDLQRYCLARYYDVRPGMMGRALSKQQNMSSMTDKLSKQEDYSGIVSRRLEYRERESRCLTETAQYVFEKTCLYVLELLSKHTMPSEDSEVTFEHPTVDPNIDIETWKIIDVSQLIIFVFDYLFENRKIVRDTTEARWTLFKIRSEVGRARIDAIEMTFPRFGRMLRNVSQAKINQEIACLNFLPLLFIIGDNISYAHRQWSIPVLLYAHDIRIIPLEVGAYNNRFGLTSYLEYMTFFPSYATRLAKIDESIKECAIAMAEFYMNTDIHSGGVMSNVITTKRLLYETYLASLCGGYSDGLLWYVPITHPSKCLVAFEVADDVVPLSVRRERILSRFPLSSRHVKGIALISVDRNQKVSVQTEGIVTHRLCKKNLLKYVCDVILFKFSGHVFGNDEMLTKLLNV

SEQ ID NO: 12 - protein, 526 aa, Bluetongue virus
MGKIIKSLSRFGKKVGNALTSNTAKKIYSTIGKAAERFAESEIGSAAIDGLVQGSVHSIMTGESYGESVKQAVLLNVLGAGDEIPDPLSPGERGIQMKIKEIEEEQRNELVRLKHGKEIAKKFGDELEEIYQFMNGEVKEEVQQEEQYKVLCKAVNSYEKLLLAENDEMRTLARALQREATERTETESKMVKEYRQKIDALKAAIEVERDGMQEEAIQEIAGMTADVLEAASEEVPLVGSGMATAIATGRAIEGAYKLKKVINALSGIDLSHLRTPKIEPTMVATTLEHRFRDIPDKELAVSILAKNNAIVANTREVQHIKEEILPKFKKIMDEEKELEGIDDKKIHPRVMMRFKVPRPQQPQIHIYSAPWDSDDVFFFHCVSHFHVNESFFLGFDLGIDVVHFEDLAAHWHALGAAQEAKGRTLNEAYKEFLNLSIGSAYTSPMHARRMIRSKTVHPIYLGSMHYDITYETLKSNAQRIVYDDELQMHILRGPLHFQRRAILGALKFGVKVLGDKIDVPLFLKNA

SEQ ID NO: 13 - protein, 526 aa, Bluetongue virus
MGKIIKSLSRFGKKVGNALSSNTAKKIYSTIGKAAERFAESEIGAATIDGLVQGSVHSIITGESYGESVKQAVLLNVLGTGEELPDPLSPGERGMQMKIKELEDEQRNELVRLKYNKEITKEFGNELEEVYDFMNGEAKEEEVVQEQYSMLCKAVDSYEKILKAEDSKMGILARALQREASERSQDEIKMVKEYRQKIDALKNAIEIERDGMQEEAIQEIAGMTADVLEAASEEVPLIGAGMATAVATGRAIEGAYKLKKVINALSGIDLSHMRSPKIEPTIIATTLEHRFKEIPDEQLAVSVLNKKTAVTDNCNEIAHIKQEILPKFKRIMDEEKEIEGIEDKVIHPRVMMRFKIPRTQQPQIHIYAAPWDSDDVFFFHCVSHHHRNESFFLGFDLGIDVVHFEDLTSHWHALGLAQEASGRTLTEAYREFLNLSISSTYSSAIHARRMIRSRAVHPIFLGSMHYDITYEALKNNAQRIVYDEELQMHILRGPLHFQRRAILGALKFGVKILGDKIDVPLFLRNA

SEQ ID NO: 14 - protein, 349 aa, Bluetongue virus; identical to SEQ ID NO: 5

SEQ ID NO: 15 - protein, 900 aa, African horse sickness virus
MQGNERIQDKNEKEKAYAPYLDGASVSTDNGPILSVFALQEIMQKIRQNQSDMAAHAPDVDGAIPEVMTIISGIKGLLEEKDYKVINAPPNSFRTIPMQSTEYVLQVNTFYERMSEIGGPVDETDPIFYALILEKLKFLKSEGAFILQGIATKDYRGAEIADPEIIGVSFQNALSHLAAIDRQIIQDTLNGMIIENGLVADRNVDVFRAAMSDPIYRIHNVLQGYIEGIQHGELRESVNWLMRLGLRKRIEFANFLTDFRRADTIWIISQRLPINANVIWNVPRCHIANLITNVALCLPTGEYLMPNPRINSITITQRITQTNPFSIISGLTPTAVQMNDVRKIYLALMFPNQIILDIKPDSSHAVDPVLRMVAGVLGHVMFTYGPIMTNITPTMAELLDAALSDYLLYMYNNRIPINYGPTGQPLDFRIGARNQYDCNAFRADPQTGRGYNGWGVVDVQRVQPSPYDHVQRVIRYCDIDSREIIDPRTYGMNMTYPIFREMLRMLVAAGKDQEAAYLRQMLPFHMIRFARINQIINEDLLSAFSPDQNFDVVLHNLIQGNFGETDPVVLEVSWASIWFAFVRRFEPIARSDLLEAAPLIEARYAAELSTMQMDVQQLRMMRARVPDTVINATPSQCWKAVLKNAPEPIKNLMNLSHSFSFVNVRDIVRWSQRDIQESLAYVLNREAWAIANDFEDLMLVDHVYIQRTMLPEPRLDDINEFRRQGFFHTNMIDGAPPIGDVTHYTYAIANLQANMGQFRAAIRRTLDDNGWIQFGGMLRNIKIKFFDSRPPDEILTAMPVYTEEERDGVRMVAFKYATTATAYFLLYNVEYSNTPDTLITVNPTFTMTKIHMRKKIVRRVRAPDVLSQVNKRLVAYKGKMRLMDVTKCLKTGVQLARPTI

SEQ ID NO: 16 - protein, 502 aa, African horse sickness virus
MGKFTSFLKRAGTATKRALSSDAAKKMYKTAGKALQRVVESEIGSAAIDGVMQGTIQSIIQGENLGDSIKQAVILNVAGTLESAPDPLSPGEQILYGKVHEIERMEKEDRVIAAHANDIEKEFGKELTIRKIVKGEEKIEELEGKEVEYVEKALDGLLKIGKNQSKRITNLYKALQTEEQLRTEDEIAMISDYKEKFGALKEAIQIEQQAANEEAIQEMLDISADVIETASEEVPIFGAAAANVVATTRAIEGGKLKEIVDKLTGIDLSHLKVAEIHPHVIGQAISKNDVDDKTLAVAIKSKVDAVDEMNQETEHVIQSILPLVKREYQKHENKFNIKIPSALKIHSEHTPKVHIYTTPWDSDKVFICRCIAPHHQQRSFMGFDLEIEYVYYEDTSVEAHIMHGGAVMIEGRGFRQAYNEFMSVAWSMPEIPELHKRRMQRNQGSHPIYMGSMDYTISFDQLSTNALQLVHNTDLQMHCLRGPIKFQRRTIMNALLFGVKIS

SEQ ID NO: 17 - protein, 347 aa, African horse sickness virus
MDAIAARALSVVRACVTVTDARVSLDPGVMETLGIAINRYNGLTNHSVSMRPQTQAERNEMFFMCTDMVLAALNVQIGNISPDYDQALATVGALATTEIPYNVQAMNDIVRITGQMQTFGPSKVQTGYAGAVEVQQSGRYYVPQGRTRGGYINSNIAEVCMDAGAAGQVNGLLAPRRGDAVMIYFVWRPLRIFCDPQGASLESAPGTFVTVDGVNVAAGDVVAWNTIAPVNVGNPGARRSILQFEVLWYTSLDRLDTVPELAPTLTRCYAYVSPTWHALRAVIFQQMNMQPINPPIFPPTERNEIVAYLLVASLADVYAALRPDFRMNGVVAPVGQINRALVLAAYH

SEQ ID NO: 18 - protein, 1040 aa, African horse sickness virus
MASEFGILLTERIFDETLEKTNCDVIITEEKKVKRKEVEGVLGYVWEETNHRFGLCEGNYDLAFSDTMYCQTKCDGSYPVFPHYIIDALRYGVMIDRNDNQVRVDLDDKRLMKIKIQPYLGEMYFSPYSTVFCKRQALALGVDDLRHSVDVRNEFEETNHQTKGVLNGNKQRALEVWKEMAYQRMRKEGSRGRCIGHDDDVMYQLIKKLRYGMMYPHHYALNARYEVSNPSAARIKDWLLKVRVNVGRAQEKAQGPLAEMARSIENDELSRQVVDQIIQYGGQFSSCSGAREDDIPINILLEYCESLTTFVHRKKRKEGDDLTARNTFRQALIKSMPTMNFKNQMKMTRGWGNYTFFSYIDRFSRIYNMNIDPNGKLWIHKTVSEQLKKKQEENRAPLTVQIDGVHIRTDETYGTVDHWVEWVVDTIMLRETEKMIKDYRFKKLKREELISGMNKLEDGLRCIVYCLILALYDYYEGDIEGFKKGTIASSIVETVSQMFPNFRSDIDKGISLKVKTEAEELFLPKNMKSSMNVGEERGYKYKFGWKDNEERVMSDYGEILTESVEILFKKLLKGEKWKIIVDDPQTYFEDDLFVDRANKIFSRGGQTVNQLISIKVNAQSNNVEGTTYFSKFVSYFRIEHFSITTAKKRTDIRDKKTEYNEFDFEDFKPACIGELGIHASTYIYQDLLVGKSRGERVKDAKELVWMDLSLANFGCSRCYDRCWPASCVEAEISLRYYLVTSIFARYLNREGLSFSKIISLKDSDRLWFPTYKHFYVAVVQKVLRDDRRLDYVLFCSRVSAITTRRATLMEFSTFKQMVGSTRLLDTLFLNFLLWIIFEQENIDVDFANKWHPLLISTEKGLRVIAVDVFNSSLTLSTSGWLPYERICSEAMDRALTADEINLKRWFVDYYMELKLERRAEPRMSFKSEALITWIGSNCGGVTDYVVQLLPVRKPKPGLLVVVYSEDGGEKWAEWALRDYLEIDGSLGLVFITRKAVKNKSKLGVRDLKINRGRVDRILISSGVYTFGNKFLFSKLLSKIE

SEQ ID NO: 19 - protein, 1049 aa, African horse sickness virus
MASEFGILYTDQIYEQTLEKTSCDVIVTKENAVKRVEIDGVLGYEWGATNHRLGLCEIEHVKTISEFMYEQIKCEGAYPVFPHYVIDALKYNKVIERNDNQVRVDRDDERLQKIKIQPYFGEAFFSPTYSSTFCKRQAIRGHVAKMRTFAKDRIDFEESSAQTKYVNGNKVKILEEWKGRTDARMLMEGQQEKCVAHEVDPIYQLIKKMRYGMMYPTHYMLSQRYRIISERRKMGIEKWLLEKVERGVQRGVAGKGNESLVRLEKLMQKEELENSVIENVIRYGSQFSTHAGEKTNDMPLSVLIKYCESLTTFVHKKNREGGDNQTARDEIRKAMVHNMPTMNHNNPMKVTKNFKNFLFFAYLDGFKRNNGVDINPNNSTWEHKKKMAEKLKDEQQKNQNRPMLVPIDGVYVSTSVEYGTVTHWVDWVVDIIMTTQVERMIKEYDFKRLERKQLISGMNKLEDGVKCYAYCLILALYDYYGDAIEGFAQGTRAGSIVETISQMFPEFRDVAEKFGIKLTIKDESEELFVQKDMNSEFLDEGEMGYKFVFGWKNTDFKVQSNYGEIVSEEVERLFKVILEGKEWSNEVEDPEDYFVDNLFNKTPDAVFERDGMDGSNRIIVKNKTTLREGQRHFSAFVSYWYTFEKVEVGRAMARIDIHDRETKFSEFDVDDYKPCSVAEMGLHSSTYIYQDLLIGPNRGEHVIDAKELVWYDIALTNYGTTRYFDQCWPSSCSTTELSMRYFLITEIFQRYRTDDRSSFADIERMRKNGYPRRNFLTYKHYYVAVIQEVFGDQRPIDVYSFCTDIFKKERRRGVLSMFPTFSTLIKSEKLIDALFLNFFLWVVFEMENVDVSFANKRHPLLISHDKGLRLIGVDLFNSALSISMGGWIPVERICHADHAARKLNADELKIKRWFIDYYMDLSLDRRAEPRMSFKYEGLATWVGSNCGGVRDYIIQELPMRKPKPGLLILVYGEDGDPKWVEWAIKDFTQIEGSLGFIYIDPISVVNKSTFRTREMKYNRGRLDRLILISSGNYTFGNKFLLSKLLSKAE

SEQ ID NO: 20 - protein, 505 aa, African horse sickness virus
MGKFTSFLKRAGSATKKALTSDAAKRMYKMAGRTLQKAVESEVGSAAIDGVMQGTIQSIIQGENLGDSIKQAVILNVAGTLESAPDPLSPGEQLLYNKVSELERMEKEDRVIETHSEKIVEKYGRELVDIRKIMRGEAQAEKLEGKEMFYIEKALKGILQIGKDQSDRITRLYRALQTEEELRTADETKIISEYREKFNALKQAIELEQQATHEEAVQEMLDLSAEVLETAAEEVPIFGAGAANVVATTRAVQGGLRLKEIIDKLTGIDLSHLKVADIHPHIIEKAMLKDAVTDSDLAMAVKSKVDVVDEVNAETEHIIDTIMPLVKKEYDKHENKFHIEIPSALKIHSEHTPKVHIYTTPWDSDKVFICRCIAPHHQQRSFMIGFDLEIEFVFYEDTSVESHVMHGGAISIEGRGFRQAYSEFMSAAWSMPAAPELHKRRLQRSLGSHPIYMGSMDYTVSYEQLVSNAMKLVYDTDLQMHCLRGPLKFQRRTLMNALLFGVRIA

SEQ ID NO: 21 - DNA, 1410 bp, synthetic construct
  misc_feature 1..1410: note = N. benthamiana optimised BTV-8 VP3
  source 1..1410: mol_type = other DNA
ttaattaatcgcgaccggtatggctgctcaaaatgagcaaaggccagagaggattaagactactccataccttgagggtgatgtgctttcttctgattctggaccacttctttctgttttcgctctccaagagattatgcagaaggttaggcaagttcaggctgattacatgactgctactagggaagttgatttcactgttccagatgtgcagaaaatcctcgatgatatcaaggctcttgctgctgagcaagtgtacaagattgtgaaggtgccatccatttctttcaggcacattgtgatgcaatctagggatagggttctcagagtggatacttactacgaagagatgtctcaggttggagatgtgattactgaagatgagccagagaagttctactccactatcatcaagaaggtgagattcattaggggaaagggatctttcattctccacgatatcccaactagagatcacagaggaatggaagttgctgagccagaagttcttggagttgagttcaagaatgtgcttccagttcttactgctgaacacagggctatgattcagaatgctctcgatggatctattatcgagaacggaaacgttgcaactagggatgtggatgtgtttattggagcttgctctgagccaatctacaggatctacaacaggcttcagggatatattgaggctgttcagcttcaagaactcaggaactctattggttggcttgaaaggcttggacagaggaagaggattacttactctcaagaggtgctcactgattttagaaggcaggatacaatttgggttttggctctccagcttccagttaacccacaagttgtttgggatgttccaaggtcctctattgctaacctcatcatgaacattgctacttgccttccaactggtgagtacattgctccaaacccaaggatttcttccattactctcactcagaggatcactactactggaccattcgctattcttactggatctactccaactgctcagcagcttaacgatgtgaggaagatctaccttgctcttatgttcccaggacagattattctcgatctcaagatcgatccaggtgaaagaatggatccagctgttagaatggttgctggtgttgttggacaccttctttttactgctggtggaaggttcactaacctcacacagaatatggctaggcagcttgatattgctctcaacgattacctcctctatatgtacaacactagggttcaggttaactatggaccaactggtgaaccactcgattttcagattggtaggaaccagtacgattgcaatgttttcagggctgatttcgctactggaactggatacaatggatgggctactattgatgtggagtacagagatccagctcc

SEQ ID NO: 22 - DNA, 2718 bp, synthetic construct
  misc_feature 1..2718: note = Plant Codon Optimised BTV-8 VP3
  source 1..2718: mol_type = other DNA
accggtatggcagcacaaaacgaacagagaccagaaagaatcaagacgacaccatacctagaaggtgacgtactaagttctgatagtggtccactactatctgtattcgcattgcaagaaatcatgcagaaagtcagacaggtccaagcagattacatgacagctactagagaagtagacttcacagtgccagatgtacagaagatcctggatgatatcaaggctctagcagcagaacaagtatacaagatcgtaaaggtcccttctatctccttcagacacatcgttatgcagtctagagatcgagttctaagagtagatacctactacgaggaaatgagccaggtaggagatgtaatcacggaagatgaaccagaaaagttctactccacgatcatcaagaaggtacgtttcatcagaggaaagggatcattcatcctacacgatatcccaacgcgagatcatcgaggtatggaagtagcagaacctgaagttttaggagttgaattcaagaacgtactaccagtcttgacagcagaacatagagcaatgatccagaacgcacttgatggttcaatcattgaaaacggaaacgtggcaacacgagatgttgatgttttcatcggagcatgttcagaaccaatataccgtatatacaacaggctgcagggatacatagaggcagttcagttacaggaactgcgaaacagtattggttggcttgaaagactgggacaacgaaaacgaattacatactcccaagaagttctgacagatttccgtagacaggatacgatttgggttctggcattacagttaccagttaacccacaggttgtttgggatgttccacgatctagtatagcaaacctgattatgaacattgccacgtgcttacctacaggagaatacatagctccaaaccctagaataagctccattaccttaacccagcgaattaccaccactggtccatttgcaatactgactggaagtactccaactgctcaacaattgaacgatgttagaaagatatacctcgccctgatgttcccaggacaaattatactcgacttaaagatagaccctggagagagaatggacccagcagttagaatggttgctggagttgttggacatttgttgttcactgctggtggtcgttttactaatctgacacaaaacatggcacgtcaattagatattgccttaaacgactacctcctctacatgtacaacaccagagtccaggttaattacggtccaacaggtgaacctcttgatttccagattggtagaaatcagtacgactgcaatgtgttcagagctgatttcgcaacaggtacaggttacaatggttgggctacaatagatgttgagtatcgtgaccctgcaccttatgttcatgctcaaagatatataaggtattgtggcattgactcccgtgagttgataaatcccaccacatatgggattggtatgacctatcactgttataatgagatgctccggatgctcgtggctgctggcaaagattctgaagcagcttatttccgttcaatgcttcctttccatatggttcgttttgctagaattaatcagattattaatgaggacctccactccgtgtttagcctccctgacgatatgtttaatgccctcctccctgatctcattgctggggctcatcaaaatgctgatcctgttgttttggatgttagttggattagcttgtggtttgcttttaatcggagctttgagcctacccatagaaatgagatgcttgagattgctcctttgattgagagcgtttatgccagtgaactttcagtgatgaaggtggatatgcgtcatttgtcattgatgcagcgtaggtttcctgatgtcttgattcaggctaggccttctcatttttggaaggctgttttgaatgactcacctgaagctgttaaagctgtgatgaatttgtctcactctcacaattttattaatattcgggacatgatgaggtgggtgttgttgccctctcttcagccgtctcttaaacttgtgttggaggaggaggcttgggctgctgctaatgattttgaagatcttatgttgaccgatcaagtgtatatgcatagggacatgttgcctgagccgaggttagatgatattgagaggtttaggcaagaagggttttattataccaatatgcttgaggccccgcccgagattgatagggttgttcaatatacttatgagattgccaggttgcaagctaatatgggccaatttcgggctgctcttaggagaattatggatgatgacgattgggtgaggtttgggggggtgcttaggactgtcagggtgaaatttttcgatgctaggcccccggatgatattttacaagggttgccctttagttatgacactaatgagaaagggggcctttcttatgccactatcaaatatgccactgagactactatattttatcttatatataatgtggagttttcaaatactcccgactcacttgtgcttattaatccgacatatactatgacaaaagtctttattaataagcggattgtcgagcgggtgagggtcggccaaattcttgccgtgcttaatcggcggtttgtcgcctataagggcaaaatgaggattatggacattactcaatcacttaaaatgggcactaaacttgccgccccgactgtctgactcgag

SEQ ID NO: 23 - DNA, 1618 bp, synthetic construct
  misc_feature 1..1618: note = N. benthamiana optimised BTV-8 VP5
  source 1..1618: mol_type = other DNA
ttaattaatcgcgaccggtatgggaaagattattaagtccctctcaaggttcggaaagaaggttggaaacgctcttacttccaacactgctaagaagatctactccactattggaaaggctgctgagagatttgctgagtctgagattggatccgctgctattgatggacttgttcagggatctgtgcactctcttatgactggtgagtcttacggtgagtctgttaagcaagctgtgcttcttaacgttatgggatctggtgaagaacttccagatccactttctccaggtgaaaggggaatgcagactaagattcgtgaacttgaggatgaacagaggaacgagcttattaggctcaagtacaacgataagatcaagcagaagttcggaaaagagcttgaagaggtttacgagttcatgaacggtgttgctaagcaagaggaagatgaagagaagcactacgatgtgcttaagaaggctgttaactcctacgataagatccttactgaggaagagaagcagatgaggattcttgctactgctctccagaaagaggtgaaagaaaggactggaactgaggctgttatggtgaaagagtacaggaacaagatcgacgctctcaaagaggctattgaggttgaaagggatggaatgcaggaagaagctatccaagagatcgctggaatgactgctgatgttcttgaagctgcttctgaagaggttccacttattggagctggaatggctactgctgttgctactggaagggctattgagggagcttacaagcttaagaaggtgatcaacgctctttctggaattgatctcactcacctcaggactccaaagattgagccaactatcgtgtctactgtgctcgatcacaagttcaaggatatcccagatgagatgcttgctgtgtctgtgctttctaagaatagggctatcgaagagaaccacaaagagatcatccacctcaagaacgaaattctcccaaggttcaagaaagctatggatgaagagaaagagatttgcggaatcgaggataagaagatccacccaaaggttatgatgaagttcaagatcccaaggactcaacaaccacagattcacatctattccgcaccttgggattctgatgatgtgttcttcttccactgcatttctcatcatcacgctaacgagtctttcttcatcggattcgatcttggaattgatttggtgcactacgaggatcttactgctcattggcatgctcttggagctgctcaagctgctgttggaagatctcttaacgaggtgtacaaagagttccttaacctcgctatcaacaacacttactcctctcaaatgcacgctagaaggatgattaggtccaagactgttcacccaatctaccttggatctctccactacgatatctcattctctactctcaggtctaatgctcagaggattgtgtacgatgaggaacttcagatgcacattcttaggggaccacttcattttcagagaagggctattctcggagctattaagcacggtgtgaagattcttggaactgaagtggatattccactcttccttaggaacgcttgactcgaggcctggcgcgcc

SEQ ID NO: 24 - DNA, 1593 bp, synthetic construct
  misc_feature 1..1593: note = Plant Codon Optimised BTV-8 VP5
  source 1..1593: mol_type = other DNA
accggtatgggaaagatcatcaagtccctgtctagattcggtaaaaaggtaggaaacgcactgacaagtaacacagctaagaagatctactccacaatcggaaaggctgctgaacgatttgctgaaagtgaaatcggaagtgctgcaatagatggtcttgtacaaggatctgtacattctctaatgacaggagaatcctacggagaaagtgtaaagcaagctgtactattgaacgttatgggaagtggagaagaactaccagatcctcttagtcctggagaacgaggaatgcaaactaaaatccgtgaactagaagatgaacaaagatccgaactaatcaggctgaagtacaacgataagatcaagcagaagttcggtaaagaactggaagaagtttacgaattcatgaacggtgttgcaaagcaagaagaagacgaagaaaagcactacgacgttctgaagaaggcagttaactcatacgacaagatcctcacagaagaggagaagcaaatgcgaatcctcgcaacagctttacaaaaagaggttaaagagcgtacaggtactgaagcagttatggttaaagagtaccgtaataagatcgacgccctcaaagaggcaatcgaagttgaaagagacggtatgcaagaagaagctattcaggaaattgcaggtatgactgcagacgttttggaagcagcatctgaagaggttccacttataggtgctggtatggcaactgcagtggctactggtagagcaattgagggtgcttataagcttaaaaaggttattaatgctttgtctggtatagatttgactcatctcagaactccaaaaattgagcctactattgtcagcacggttctcgatcataagttcaaagacataccggacgagatgttggcagtgtcagtgctttctaaaaatcgtgctattgaggagaatcataaagagattatacaccttaaaaatgagatactccctaggttcaagaaagccatggatgaggagaaagagatttgcggcatagaggataagaaaattcaccctaaagtcatgatgaagtttaaaattccgaggacccagcagccacagatacacatatatagcgctccttgggatagcgatgatgtctttttctttcactgtattagccaccatcacgctaatgagagctttttcattgggtttgatttgggcattgatcttgtgcactatgaggatcttacggcccattggcatgctcttggcgctgctcaagctgctgtcggcagatctcttaatgaggtgtataaggagtttttgaacttggccattaataatacctattcatcacagatgcatgccaggaggatgattaggtcaaaaaccgtgcatccaatatatttggggtcacttcattatgatatttcattttctaccttgcggtctaatgcccagaggattgtctatgatgaggagttgcagatgcatattcttcggggccccttacattttcagagacgggccattttaggggccattaagcatggggtgaaaattttagggaccgaggtggatattcccttatttttaagaaatgcctgactcgag

SEQ ID NO: 25 - DNA, 1087 bp, synthetic construct
  misc_feature 1..1087: note = N. benthamiana optimised VP7
  source 1..1087: mol_type = other DNA
ttaattaatcgcgaccggtatggatacaattgctgctagggctcttactgttatgagagcttgcgctactcttcaagaggctaggattgtgcttgaggctaacgtgatggaaattctcggaatcgctatcaataggtacaacggacttactcttaggggtgttactatgaggccaacttctcttgctcagaggaacgagatgttcttcatgtgcctcgatatgatgctttctgctgctggtattaacgtgggaccaatctctccagattacactcagcacatggctactattggagttcttgctactccagagatcccttttactactgaggctgctaatgagattgctagggtgacaggtgaaacttctacttggggaccagctagacaaccatacggatttttcttggagactgaagagacttttcaaccaggacgttggtttatgagagctgctcaagctgttactgctgttgtttgcggaccagatatgattcaggtgtcccttaatgctggtgctagaggtgatgttcagcagattttccagggtaggaacgatccaatgatgatctaccttgtgtggagaaggattgagaatttcgctatggctcagggaaattctcaacaaactcaggctggtgttactgtttctgttggaggtgttgatatgagagctggaaggattattgcttgggatggacaagctgcacttcatgttcataacccaactcagcagaatgctatggttcagattcaggtggtgttctacatctctatggataagactctcaaccagtacccagctcttactgctgagatcttcaacgtttactccttcagggatcatacttggcatggactcaggactgctattctcaacaggactactcttccaaatatgctcccaccaatcttcccaccaaacgatagggattctatccttactttgctccttcttagcactcttgctgatgtgtacactgttcttaggccagagttcgctattcatggtgttaatccaatgccaggaccacttactagagctattgctagggctgcttatgtgtgactcgaggcctggcgcgcc

SEQ ID NO: 26 - DNA, 1062 bp, synthetic construct
  misc_feature 1..1062: note = Plant Codon Optimised BTV-8 VP7
  source 1..1062: mol_type = other DNA
accggtatggatacgatcgctgctcgtgctctaactgtaatgagagcatgtgctacactacaagaagcacgaatcgtactggaagctaacgtcatggaaatcctgggaatcgccataaatcgatacaacggtctgacgctacggggagtaacaatgagacctactagtcttgcacaaagaaacgaaatgttcttcatgtgcctcgacatgatgctctccgcagctggaattaacgtcggtccaatatccccagattacacacaacatatggcaacaatcggtgtgctcgcaacacctgaaatccctttcactacagaagcagctaacgaaatagcacgtgtaaccggagagacaagtacatggggaccagcaagacaaccatatggattcttcttggagacagaggagacatttcaaccaggtagatggtttatgagggctgctcaagcagttactgctgttgtttgtggaccagatatgatacaggtgagccttaacgctggtgcaagaggtgacgttcaacaaatatttcagggtagaaacgaccctatgatgatatacctcgtgtggaggagaattgagaattttgctatggcccagggcaatagccagcaaacccaagctggtgttactgtttctgtgggcggcgttgatatgagggcagggcgaattattgcttgggatgggcaggctgcacttcatgttcataatccaactcagcagaatgctatggttcagattcaggttgttttctatatttcaatggacaagaccttgaatcagtacccggctcttacggctgagatttttaatgtctattcatttcgtgatcacacctggcacgggttgaggaccgctattttgaatcggaccactttgcctaatatgttgcctcctatttttcccccgaatgatagggattctattcttactcttttgcttttatctactttagccgatgtctatactgtgttacgtcctgagtttgccattcatggcgtgaatccgatgcccgggcccttaactagggccattgcccgggccgcctatgtgtgactcgag

SEQ ID NO: 27 - DNA, 2923 bp, synthetic construct
  misc_feature 1..2923: note = N. benthamiana optimised BTV-8 VP2
  source 1..2923: mol_type = other DNA
ttaattaatcgcgaccggtatggaagaactcgctatcccaatctacactaacgttttcccagctgagcttcttgatggatacgattacatcatcgatgtttcttctagggttgaagaagaaggcgacgagccagttaagaggcatgatgtgactgagattcctaggaactccatgttcgatatcaaggatgagcacatcagggatgctattatctacaagccagtgaacaacgatggatacgttttgccaagggtgctcgatattactctcaaggctttcgatgataggaagcgtgttgttcttaacgatggacactctgagtttcacactaagactaactgggtgcagtggatgattgatgatgctatggatgtgcagccactcaaagttgatattgctcacactaggtctaggatttctcacgctctcttcaattgcactgtgaggcttcattctaagaaggctgatactgcttcttatcacgttgagccagtggaaattgaatcttggggatgcaatcacacttggctttctaggattcaccaccttgttaacgttgagcttttccactgttctcaagaggctgcttacactcttaagccaacttacaagatcatttccaacgctgaaagggcttctacttccgattctttcaacggaactatgatcgagcttggtaggaatcatcagatccagatgggagatcaaggataccagaagcttaaagagggacttgttcaggttagaatcgagggaaagactccacttgttatccaggaagagatcactgctctcaacaagattcgtgagcaatggattgctaggaatttcgatcagcgtgagattaaggttttggatttgtgcaggcttctttcaactatcggtaggaagatgtgcaatactgaagaggaacctaagaacgaggctgatctttctgttaagttccagatggaactcgatgagattttcaggccaggaaataacgagaggactaacattatgggaggtggagttcatagaaagaacgaggacaggttctacgtgctcattatgattgctgcttccgatacaaacaagggaaggatttggtggtctaatccatatccatgtcttaggggtgctcttattgctgctgaggttcagcttggagatgtgtacaaccttctccgtaattggttccaatggtctgttaggccaacttacgttccatacgataggaacagagagtccgataagtacatctactccaggatcaaccttttcgattctactcttaggccaggtgataagattgtgcactgggagtacaagcttttgaacgaggttagagaggtgtcaatcaacaagggaaacgagtgtgatttgttcccagaggatgaagagttcactactaagttccatgaggctaggtatactgagatgaagaaccagattattcagtctggatggaaccagagggatttcaagatgcacaagatccttgaggatggtgctaacgtgctcacaatcgattttgagaaggatgctcacattggaactggatctgctctttctctccctgattactacaacaagtggattatcgctccaatgttcaacgctaagctcaggattactgaggttgtgattggaactgctcacactgatgatccagctgttggaagatctgctaaggctttcactcacgatccattcgatcttcaaaggtactgtctcgctaggtattatgatgttaggccaggaatgatgggtagagctttgtctaagcagcagaatatgtcctccatgactgataagctttccaagcaagaggattacgctggaattgtgtcaagaaggcttgagtacaaagagagagagaaccgttgtcttactgagactgctcagtacgttttcgaaaagacttgcctctacgttttggagcttctttctaggcatacaatgccatctgaggattctgaagtgactttcgagcacccaactattgatccatctgtggatatcgagacttggaagatcattgatgtgtcccagctcattatcttcgtgttcgattaccttttcgagaaccgtaagatcgttagggatacaactgaagctaggtggactcttttcaagattaggtctgaggttggaagggctaggattgatgctatcgagatgacttttccaaggttcggaaggatgcttaggaatgcttctcaggctaagatcaaccaggatatcgcttgccttaacttccttccactcctcttcattatcggtgataacatctcttacgctcataggcaatggtctattccagtgcttctttacgctcacgatatcaggattatcccacttgaggttggagcttacaataacaggttcggacttacttcttaccttgagtacatggctttcttcccatcttatgctactagggtggcaaagattgatgagtccatcaaagagtgcgctattgctatggctgagttctacatgaacactgatattcactctggatccgtgatgtctaacgtgatcactactaagaggcttctctacgagacttaccttgcttctctttgcggtggatactctgatggacttctctggtacttgccaattactcacccatctaagtgccttgttgctttcgaagttgctgatgatgttgtgccactttctgttagaagagagaggattctttctaggttcccactttcttcaaggcacgtgaagggaattgctcttatttccgtggataggaaccagaaagtttctgttcagactgagggaattgtgactcataggctctgcaagaagaaccttctcaagtacgtttgtgatgtgatcctcttcaagttctctggtcacgttttcggaaacgatgagatgcttactaagctcctcaacgtttgactcgaggcctggcgcgcc

SEQ ID NO: 28 - DNA, 2889 bp, synthetic construct
  misc_feature 1..2889: note = Plant Codon Optimised BTV-2 VP2
  source 1..2889: mol_type = other DNA
atggatgaattaggaataccaatctacggaaggaattacccagaacacttactcaaaggatacgaatttttaatcaatacaggagttaggtatccatctcaaggaggtagacatgatgtttcaaagattcctgaaatgttcgcttatgatattaaggatgagggaataagagaagcattgaaatacatgccaactaggaatgatggagttgtgttgcctagaatcgtggatattagtttaaagggttacgatatcaggaaatccgttattgaagctaataagagtaactccttccacactgatacaaattgggttcagtggatgattaaggattctatggatcaacagccattgaaaatctcaattgatgaagagcatagtagagttgtgcactccctttttaactgtcaggttaagatcgatgctaagaaagcagatactctctcttatcatcttgaagctattgaggatgctgaaaaagcatgtttgcatacaaggggtcaattgtgcaatcatcttgctaggatggatcttttgcatgctgcacaagaaattgcttatgctattaagcctacttaccagcttattgtgcactcagagagggcttctacatcagataactttgaactcggaagacaagatgttattactcttagaaggggacataggatacagatgggagatgaggcatatactaagttgatggaaagacttgttaggttgacagtgcagggtaacgttccaagaaaaatccaatctgaaattgagcagcttgaagctattagaactaggtgggcaacaggaagatacgatcctgctcacatcaacagtcaagatctctgtaggattctttccagaattggtaggatcatgcttgatcaagaagctgagccagttgatgaagatagtctttccttgagattccagagggcactcgatgagaagtttaggcttaacgattctgagagaaacaaaatcttcgaacctaagtcacatagaaaggatgaagataggttttatgttttactcgctatagctgcatctgatacttataattcaagaatctggtggagtaacccatacccttgcttgaggggaactttaattgctgcagagacaaagctcggagatgtttattttactcttaggtcctggtacgattggtctgtgagatcttcatatgttccaagagaaagggagagagaaacagagaagtacatattctctaaaatcaatttgttcgattacgaagctggacctagttccaaggttattcattgggagtatcagctttacaaaagggagagagttgtgactttggaaagaggtaacccatgtgatctttatcctgatgaggatgatgaagtgattataacaaagttcgatgatgcaaagtactcagagatggttggagaaatcattgatggaggttggaatgatgaagagttcaagatgtataagcttttgcaagagaagggtaatgttttgactatagatttcgaaaaggatactaagctcaactctacatcagaggttgtgcttccagattattacggaaagtggattgttgctcctatgttcaactctaaaatgagaattattgaaactgagattgcaacaaacaggtcagatgatccaatgataaagagaactttgaaaccaatgacagatgatcctgttgaattgcaaagatatactttagctaggtactacgatattagacctggactcatgggtagatctcttaataggactcaaacacagtcaacattcgatgcaaaggttagtgagctttccgattacgaaaaagttgtgtcaagatttggagtgataaagaaaccaactaggccttgcgttactttgacaggtagatacatcttagaaaagtacagtctcctccttatcgatatcttgaagtaccacactgaggttgaagagaacccacaagaagagttcacacatcctagaatagatcttcaattcaagtttaatggtaacactttatctgatctcaaccaaacagttgtgtttattgttgattatcttcacgaaaagagaaactacgttaggagtgtgtatgaggctagatacattatatccaggataagatcttcaacaggtgctgcaaggatgagtatcttggaattctacttcccaacttttgctaggctcatatctaatgcaagagagcctacatacgtgaaagatcttatggcattgaatttcttaccattgctttttattgttggagataacatgatctataagcatagacaatggtcaattccactccttttgtacactgatagggttaaagtgatacctttggaagtgggaagttccaataacagacagggtctcgttagttatcttgagtatatgtttttctttccttcccttgctgataggacatcaaaggtggatgagagtatgataaaggtttccaaggaagttgtgaactactacatgaagactacaatctctgaaggaggtgttaatttgaacgttgtgtctactaagtcactcctctacgatatatatctttcttctgtgtgtggaggtgtttcagatggtgttgtgtggtatttgccaatcacacacccttacaaatgtgttgtggctattgaagtgtgcgatgatagagttccagcaaggcttagatgcgataggttaagactcaggttccctttgtctgctcaacatcttaagggaatagttgtgattcaaattaatgaggagggaggttttgatgtttatactgagggtattgtgacacacagggtttgtaagaaatcacttttgaagcatgtttgtgatatcgttttactcaagttccacggtcacgttttcggtaatgatgagatgcttacaaagttattaaatgtgtga

SEQ ID NO: 29 - DNA, 2890 bp, synthetic construct
  misc_feature 1..2890: note = Plant Codon Optimised BTV-3 VP2
  source 1..2890: mol_type = other DNA
accggtatggaggaactggtaatccctgtaattagccggcaattcgataagaagctggtaggtagatacgaatacgttatcgaactagcagaaccagaacaagatgaatggactaaccatgacgtaacacaaatccctggacgtagaatgtttgatgtagcacaacaaggaatccgagaagctataatctacaagccattggataacgacggtgaagttttgcctagaatactagacatgagtatcgcttgttacgatatgagaaagacgatgatgaagaaggagggtgttgatttcgtgtctaacacaagatggcttgaatggatgatccaagactctatggatgttcaaccacttagagtacaaatgaaggaggaccattctacagtgcaatatgacatgttctccgctaaggtacatatcgacagtcgaaaagctgatactacttcctaccatgctattgcagttgaaacaaaggcagagagaaagtgttgtcatgtaagaacagaagtctggaattccgtagtcagaaatcatcttttcaacaccgcacaagagagttgttacacgttcaagcaaacgtacgaactgatcgtaaactccgaacgactaagtacagaggaggaatttcgagtaggagctccacagtttcacactatacaaagaaatcaccgaatgcaactaggagataacgcttatgataagttcctaaagggattggtacaactgcgagtttctggaactactccagcaaagattagagatgaagttgctgctcttgatgttattagggataactggataagaggatcattcgatcgtagtcatatcaaaagcctggaactgtgtagacttctaagttctattgggcgtaagatggttaacatggaggaggagcctaaagatgaaaaggatctgtccgttaaattccagttccgactagatgagaagttctccccaaatgatccagaaagaaacgttatctttaccagtaagacccatagaaccaacgaagatcgattttacgtcctactggtcatcgcagctagtgatacaaataacggaagagtttggtggtctaatccatacccttgtctaagaggagcacttattgcagcagaatgtaaacttggtgatgtttaccatacgctgagatcaaagtatgaatggggagttagaccaacttacaaacctaaggatttggaacgtgaacgtgagaaatacgttgttggaagggtgaatctgtttgatctggaaggagagcctgcaactaaagttatacactgggaatacgaactcatttcacctacttacagtgttagtaaccacaaaggtaatcactgtgatctctacccagatgatgttgaaataaccacgaagtttaatgaggatcggtaccgtgagatgatacagtccgtgattgatgatggttgggatcaaaagaacttgaagatgtacaagatattggaggaagaagggaatcctttgctctatgatctcgaaaaggacataaagctcgattcacagagtcaagttgttttcccatcatactataataagtggacccacgctccaatgttcaatgcaagagttaagccttgtgatatcgagttggctgagcgaaaaaatgatgatcccttcgttaagcgtacattgaagcctataaaggctgattgtgtcgatctcttgaggtatcatatgtcacattactacgacctcagaccttcacttaagggtgtttcactttcaaacaagcagacaccatcaggtatacatcaggctcttgttcaggatgatctttattcccgtcttttaaggcgtcgtgatgtggatttagactattcatctccttgcccgattataactaattattttctcttggagaaattccacatactcatcttgaccatcatggagaaacactattgggaactcgacgactcagacgatgtttatgagttccctaagatcgacgcaagcgcttttgaggttgatggtactctttatgatattagccagacaattgtgcatatgtatgacaggttcttcgagaaaaggagagtgctccgttctatagatgaatccaggtggattttgcatctcatcaggatttctcagggtagggaaaggttagaagttattgagaggttttttcctaattatgggaaagccatgaggcagagggattttaaaaaagtccgggatgttatgtttcttaattttttgccgttcttttttcttaccggcgacaatattagctatgagcaccggcagtggagcattccgattattttgtatgccgacaaattgcggattcttccgattgaggtcggtgcccattataataggtttggtgttacttgcatacttgagttattaaatttttttccgagctatgaaaaaagggaggaaaaattggaagaggacattgtgttgtgcgccgacgccattgtgaatttttatcttcagaccacaattagcaatggcggcgtgcagacctctattgtgtctactaaggcccttttatatgagatgtatttgtcttctatttgcggcgggtattctgagggtgtcctttggtatcttcccattacacaccccgtgaaatgcttagttgctttggaggtgtcagatgccttggtgggtgctgatgtgaggattgataaaattaggaggcggtttcccttatctgctaaacatcttaaggggattgtccagatttctgtgcatcccaatcggacattttctgtcacaacttgcggcattgtcaaacacaaagtgtgcaaaaaaactttattaaaacatcggtgcgacgtcattttacttcagactccagggtatgtgtttggcaatgacgagcttttgacaaaattattgaatatttgactcg

SEQ ID NO: 30 - DNA, 2883 bp, synthetic construct
  misc_feature 1..2883: note = Plant Codon Optimised BTV-4 VP2
  source 1..2883: mol_type = other DNA
accggtatggaagaattcgtcatcccagttttcagtgaacgggaaatcccatatgcactaatcaaccagtacccattagcaatccaaacagatgtacgggtagttgatgtagatggtaaccataacctagtcaagatcccagaaagtgatatgatcgacgtacctaaactagacatcgttagtgcacttaactacaagccaactagaaacgacggaatagtagtcccaagactacttgatatcactctaaaggcatacgacgatagaaagtccgttaagaacgcaagaggagtagatttcatgacagacgcaaaatggatgaagtgggcaatagatgatagaatggacatccaaccattgaagatcacactggatgaacattactccgtaaaccatcaattgttcaactgcatcgtcaaggcaaagacagcaaacgcagatacaatctactacgattacttcccattggaagaccgagctaaaaagtgtaaccatacaaacctggaactactacgaagtctgacaacaatcgaagcattccatatcctacaaggagcagcttatagtctaaagtccaactacgacctgatagctaattccgaaagagaatccctggaagaatcttaccctataggtagtgaaaagtgggttcatttgacaagacgaacaaagatcggaaactctggacttagttacaatcgtttcatcagcagtatggtacaggtcgttgtaagaggaaaggttcctgatatcatcagaggtgaaatcacacaattgaaccgaattaggacagaatggattggagcaagttacgacagaactagaatacgtgctctggaactttgtaatatactgtccgctataggtagaaagatgatggacacacacgaagaaccaaaggacgaaatggacctgtcaacaagattccagtttaagttagacgaaaagttcaatacgagcgactccgaacatgtcaatattttccgaaccagtggagctgctacaaatgaaggtagattctacgctctgatagcaattgctgcaactgatactcaaaagggacgagtttggagaactaatccatatccttgtttaagaggtgctttaatagctagcgaatgtgaactgggagatgtttactacactttaaggcacgtatatcagtggagtttgcgaccagaatacggacaacgagaacaacaattggaagataacaagtacgtattcggtagagtcaatctcttcgactcagacttggcagttggagatcaaattatacactggcaatacgaaattaccgagccagttaagactacttacgacgatggatacatatgtaatccggaagaagaggacgatgagctcctttgtaaaattgacgacgagcgttacaaagagatgatggagcgtttaatagagggaggatgggatcaagagaggtttaaattacattccattctcactgagcctaatcttctcactattgatttcgagaaggatgcttacctcaattcccgttctgagttggtattccctaattacttcgataagtggattaatagcccgatgtttaatgcccgtttaaggattacccacggtgagataggttcttcaaagactattgatccttggaatcgtagagtggtttacggttatgtgaagactagcattgagagcttagattatgccctcggtcgttattatgatattcggctccaactctttggggatactctttctcaaaagcaaactcagagcgctgtttttacttatctcagcgagcaagatgattttcctgctcttaccaattatagcaagggtgaggctgtttgtcctcatgctggtggtgctgtttatacgtttagaaaagttgctctttctttgatagccaattatgagaagctctctcctgagatgcatgagggtttggagcatcaaatgtatgttcatccttcagctaataccacctatcaaaagcaagttaaggatatgaaggatttttcacagctcatttgctttattattgattgcatttttgagaagagggtgcagattaggggcgtcggggaggctaggcgtataatatatcttattcagaattctacgggctctcagcgtcaggaagttcttaagaaaacctttcctaacttctttatgaggatttttaaattgagggaggtcaaacggatttgcgatctttctgtgattaattttcttcctttgttgtttcttgtgcaggataatatttcatattggcacaggcagtggtctgtgcccatgattctttttgatgatgccgtgaggcttattcccgtggaggtgggcgcttatgccaataggtttgggcttaaatctttttataattttgtgcggtttcacccgggcgattcaaagaaaaaacaggatgccgatgatatgcacaaagagtatggcgtggcttgttttaaatattatatgaataccaaaatttcacagggcggggtgaatgtgccggtcgtcacgtctaaattggatacgcttaaaattcacttggcctcattgtgccttgggttggccgattctattgtctataccttgcctgttgcccatccgaaaaaatgtattgtgcttattgtggtgggcgatgataaattggatccccaggttaggtcagagcaggttttatctaaatattattattcaaggcggcatatttgcggtattgtggccgtttctgttgggcaggaggggcagttgcaggtttattcatctggtattgttaggcataggatttgtgagaaatcaattcttaaatataagtgcaaagtggttttggttcggatgcccggccacgtttttgggaatgatgagcttatgaccaaattacttaatgtttgactcgag

SEQ ID NO: 31 - DNA, 2898 bp, synthetic construct
  misc_feature 1..2898: note = Plant Codon Optimised BTV-8 VP2
  source 1..2898: mol_type = other DNA
accggtatggaagaactggcaatcccaatctacacaaacgtattccctgcagaactactagatggatacgattacatcatcgatatctcctccagagtagaagaagaaggagatgaaccagttaagcgacatgatgttacagaaatccctagaaactccatgttcgatatcaaggatgagcacatcagagatgcaatcatctacaagccagtaaacaacgatggttacgtactgccaagagtacttgatatcacactgaaggcattcgatgatagaaagagagtaatcctcaacgatggacacagtgaattccacactaagtcaaactgggtacaatggatgatcgatgatgcaatggatgtacaaccactaaaggtagatatcgcacacactagaagtcgaatcagtcatgctttgttcaactgcactgtaagactacacagtaagaaggcagatacagcaagttaccatgttgagccagttgaaatcgaatcacgaggatgtaatcatacatggctaggtagaatccatcatctagttaacgttgagctgttccattgctctcaagaagcagcatatgcacttaagcctacttacaagatcatctctaacgctgaacgagcatctacaagtgatagtttcaacggaacaatgatcgaactaggaagaaaccaccaaatccaaatgggagatcaaggataccaaaagctgaaggaaggactagttcaagttagaatagagggaaagacaccactggcaatacaagaagaaattacggcattgaacaagattagggagcaatggattgcacgaaacttcgatcaacgagagataaaggttctggatttgtgccgattattatccacaattggcagaaagatgtgcaacacagaggaagaacctaagaacgaagctgatctgtctgttaagttccaaatggaactggatgaaattttcaggccaggaaacaatgagagaacgaatattatgggcggaggtgttcacagaaagaatgaggatagattctacgtcctgattatgattgctgcttccgatacgaacaagggaagaatttggtggtcaaatccatacccatgtttgagaggtgctttaattgctgctgaagttcaactcggagatgtttacaatctcctcagaaattggttccaatggtctataaggccaacatacgttccatacgatagaaatcgagagagtgataagtacatatacagcaggattaatctcttcgactccaccctcaagcctggagataagatagttcactgggaatacgaactcttgaatgaagtgcgtgaagtttccattagtaagggtaatgagtgcgatctcttccctgaagatgaagagttcactacaaagtttcacgaagctagatacacagaaatgaagaatcagattattcagtccgggtggaatcagcgtgattttaagatgcacaaaatacttgaggacggtgctaatgtgctcacgattgactttgagaaagacgctcacattggtacgggttctgcactttctcttcctgattattacaacaaatggattattgctcccatgtttaatgccaaactccggattaccgaggttgttatagggacagcacatacagatgatcctgctgttggtagatctgctaaagcttttacgcatgatccttttgatctccagaggtattgtttggcaaggtattatgacgttcgtcctggtatgatgggtagggctttatctaaacagcagaatatgagttctatgactgataaactttccaaacaggaggattatagcggtattgtgagccgtcgtcttgaatatcgtgaaagggaatcaaggtgtcttacagaaaccgctcagtatgtttttgaaaaaacctgcctttatgtgcttgagttgttgagcaaacataccatgcctagcgaagatagcgaagttacctttgagcatcctactgttgatcctaatattgacatagagacctggaaaataatagacgtgagccagcttataatttttgtctttgactatctttttgagaatcggaaaattgtccgggacaccaccgaggcccgttggactctttttaaaattcggagcgaggttggtcgtgctaggatagatgctattgagatgacttttcctaggtttggtcgtatgttgcgtaatgttagccaggctaaaattaatcaggagattgcctgtttgaatttcttgccgcttttgtttataattggcgacaatataagttatgcccatcggcagtggtctatacccgttcttttgtatgcccatgacatacgtattattccgcttgaggtgggtgcttataataatcggtttgggttgacctcatatttggagtatatgaccttctttccgtcatatgccactaggttggctaaaattgacgagtcaattaaggagtgcgctatagctatggctgagttttatatgaatactgacattcattcaggcggggtcatgtctaatgtgattactactaaacggttgctttatgagacttatttggcctctttgtgtggcggctattctgacggtcttctttggtatgtgccgattactcatccctcaaaatgtcttgtggcctttgaggtcgccgacgacgtggtccccttatctgtgaggagggagaggattttatctaggtttccgttatcatctaggcatgtcaaagggattgccttaatttcagtggacaggaatcagaaagtctcagtccagactgaggggattgtgactcatcggttatgtaaaaagaatttgttaaaatatgtctgtgacgtcattttgtttaaattttcagggcatgtgtttggcaatgacgagatgcttactaaattacttaatgtgtgactcgag

SEQ ID NO: 32 - DNA, 1593 bp, synthetic construct
  misc_feature 1..1593: note = Plant Codon Optimised BTV-3 VP5
  source 1..1593: mol_type = other DNA
accggtatgggaaagatcatcaagagcctatccagattcggtaagaaggtcggtaatgctcttaccagtaacacagctaagaagatctactccacaatcggaaaggcagctgaaaggttcgcagaaagtgaaatcggttctgctgctattgatggactagtacaaggatcagtacattcaatcatgacaggagagagttacggagaaagtgtaaagcaggcagtactactaaacgtcttaggtgctggagatgaaatcccagatccactttcaccaggtgaaagaggtattcaaatgaagatcaaagagatcgaggaggaacagagaaacgaactggtacgattgaagcatggtaaggaaattgcaaagaagtttggcgatgagctagaggaaatctaccaattcatgaacggtgaagtcaaggaggaggttcaacaggaagaacagtacaaagtgctgtgtaaggcagttaacagttacgagaagttgctgttagctgagaacgatgagatgagaacactggctcgtgcacttcaacgagaagctacagaacgaacagaaacagaatctaaaatggttaaggagtaccgtcaaaaaattgacgcattaaaggccgcaatcgaggttgaaagagacggaatgcaagaagaagcaatacaggagattgctggaatgactgcagatgttttagaggctgcttcagaagaagttcctcttgttggttctggaatggcaactgctatagctactggacgtgcaattgaaggtgcttataaattgaagaaggttataaatgccctgtcaggtattgacctctcacacttgcgtactcctaaaattgagcctactatggtcgcaactactttggagcatcgtttcagagatattccagacaaggaactcgctgtttctatactcgcaaagaataatgccatagtggctaataccagggaagttcagcacattaaagaggagatacttcccaaattcaagaaaataatggacgaggagaaagagctcgaaggcatagacgacaaaaaaattcaccccagggtcatgatgaggtttaaagtgcctaggccacagcaacctcagattcacatttattccgccccatgggattctgatgatgttttcttttttcactgcgttagtcacttccacgttaatgagtctttctttctcgggtttgacctcggtatagatgtcgtgcattttgaggatcttgcagctcattggcatgcacttggggctgctcaagaagctaaagggaggactttgaatgaagcttataaggaatttcttaatcttagcattggctctgcctatacgtctcctatgcatgcccggaggatgattagaagcaaaaccgtgcatcctatttatcttggctccatgcattatgatattacctatgagacgttgaaaagcaatgcccaaagaattgtgtatgatgatgagttgcagatgcatattcttcgggggccgttacattttcagcggagagccattttgggggccttaaaatttggggttaaagtgttgggcgataagattgatgtgccgctttttttgaaaaatgcctgactcgag

SEQ ID NO: 33 - DNA, 1593 bp, synthetic construct
  misc_feature 1..1593: note = Plant Codon Optimised BTV-4 VP5
  source 1..1593: mol_type = other DNA
accggtatgggtaaaatcatcaagtccctctccagattcggaaagaaggtaggtaacgcactatcttcaaacacggcaaagaagatctactctacgatcggaaaggcagcagaacgatttgcagaatctgaaattggagcagctactatagatggacttgtacaaggatctgtacatagtatcatcacaggagaaagttacggtgaaagtgtcaagcaagctgtactactaaacgtcctgggaactggagaagaattgcctgatccactttctccaggagaacgaggtatgcaaatgaaaattaaggaactggaagatgaacaacgaaatgagctagtcagactgaagtacaacaaggaaatcaccaaggaattcggaaacgaactggaagaagtctacgatttcatgaatggggaagctaaggaagaggaagtagtccaagaacaatacagcatgctctgtaaggctgttgatagttatgagaagatcctgaaggcagaagattccaagatgggaatcctcgcacgagctttgcaaagagaagctagtgagagatcacaagatgaaattaagatggttaaggagtataggcagaagatcgatgctctcaagaatgctatcgaaatcgagcgtgatggtatgcaagaagaagcaattcaggaaatagcaggtatgacagcagatgttttggaggctgcatcagaagaagttccattgataggtgcaggtatggctacagctgttgcaactggtcgtgcaattgaaggtgcttacaaattgaaaaaagttattaatgccctctccggtattgacctctctcatatgaggtcacctaaaatagagccaacaattattgccaccactttggagcatagattcaaagagatacccgatgagcaattggcagttagtgttcttaacaagaaaaccgctgtgaccgataattgcaatgagattgctcacataaaacaggagatacttccgaagttcaaacggattatggacgaggagaaagagattgagggcatagaggacaaagtgatacaccctcgtgttatgatgaggttcaaaattccgcgtactcagcagcctcagattcatatatatgctgctccttgggattctgatgatgtgtttttctttcactgtgtgagccatcaccatcgtaatgagtcattctttttggggtttgaccttggtatagacgtggttcactttgaggacttgacatcacactggcatgctttgggccttgctcaggaggcttctggcagaactttaacagaggcttatagagagtttcttaatctttcaatttcttctacatattccagcgctattcatgccagaagaatgattaggagcagggccgttcatcctatttttcttggcagcatgcattatgatattacttatgaggccttgaaaaataatgcccagcggattgtgtatgacgaggagcttcagatgcatattcttagggggccattacactttcagaggagggccattttaggggcccttaaatttggggttaaaattttaggcgataaaattgacgtgcccttatttttacggaatgcctgactcgag

SEQ ID NO: 34 - DNA, 1062 bp, synthetic construct
  misc_feature 1..1062: note = Plant Codon Optimised BTV-4 VP7
  source 1..1062: mol_type = other DNA
accggtatggacacaatcgcagcaagagctcttacagtaatgagggcatgtgctacactgcaagaagcaagaatcgtccttgaagcaaacgtaatggagatcctaggaatcgccatcaacagatacaacggtctgacactgagaggagtcacaatgagacctacaagtctcgcacaaagaaacgaaatgttcttcatgtgcctcgacatgatgctctccgcagctggaattaacgtaggacctatctcccctgattacacacaacatatggctacgataggagtgctcgctactccagaaatacctttcacaacggaagctgcaaacgaaattgctagggtaacgggtgaaacctcaacttggggtccagcaagacaaccttatggtttcttccttgagactgaggagactttccaaccaggaagatggtttatgagggctgctcaagcagttactgcagttgtttgtggtcctgatatgatacaagtgagtcttaacgctggagctcgtggggatgttcaacaaatttttcagggtcgaaatgacccaatgatgatatacttggtgtggcggcgaatagagaattttgctatggctcagggcaatagccagcaaacccaggctggtgttactgttagcgttggtggggttgatatgcgagcagggcgtattatagcatgggatgggcaggctgctttacatgttcataatccaacacagcagaatgctatggttcagattcaggttgtgttttacatttctatggacaagacccttaatcagtatcccgccctaaccgctgagatttttaatgtctattcttttagggatcacacctggcacggcttgaggaccgctattcttaatcggactactctaccgaatatgttgccgcccatttttcctcccaatgatcgggattctattcttactttgttgttgttgtcaacattagccgatgtgtatactgtgttaaggccagagtttgccattcatggcgtcaatccgatgccaggccctttaactcgtgccattgcccgtgccgcctatgtgtgactcgag

SEQ ID NO: 35 - DNA, 2719 bp, synthetic construct
  misc_feature 1..2719: note = Plant Codon Optimised AHSV1 VP3
  source 1..2719: mol_type = other DNA
ggtaccggtatgcaaggtaacgaacgtattcaggataagaatgagaaggaaaaagcttatgcaccttatctggatggagcttctgttagcacagataatggtccaattttgtctgtttcgccttgcaagaaataatgcaaaagattaggcaaaatcaatctgatatggcagcacatgcaccagatgttgatggggccatccctgaggtcatgactattattagtgggatcaagggccttttggggaaaaggattataaagttataaacgcacctccaaattcattcagaaccattccaatgcagtctaccgagtatgtgcttcaagtgaatactttctatgagcgaatgtcagagattggaggtccagttgacgaaactgacccgataggattctacgctttgatactcgaaaagcttaagtttttgaaatctgagggagatttatcctgcaaggtatagctactaaagactatcgaggtgcagaaattgcagatcctgaaattataggcgttagtttccagaacgcattgtcccatctagcagctattgacaggcaaatcattcaaatacacttaacggtatgataatagaaaacggcttggttgccgatcgtaacgttgatgtatttcgagcagctatgtctgatcctatatacaggatccataatgtgttacaaggttacatagagggtatcagtatggtgaattgcgtgagtctgtgaattggttgatgaggctcggtctgagaaaaaggatcgagtttgctaacgattttctcactgatttcaggagggctgataccatatggataatcagccaaagctcccaattaatgctaatgttatatggaatgtacccaggtgccatattgccaacctcattactaatgtagcattgtgtctacccactggggaatatctaatgccaaaccccagaatcaactctattcaattactcaacgtatcacacagacaaatccattctcaattatctcaggcttgacaccgactgccgtgcagatgaacgacgttcgaaagatttatcttgcattaatgttccctaatcagattatactgatataaaaccagattccagccacgctgttgaccctgtactgagaatggtagctggagtattgggtcatgtcatgttcacttatggcccaattatgactaacatcacccctactatggctgaacttctgatgctgcattatccgattacttactgtatatgtacaacaataggataccaatcaattatggacctaccggacaaccattagattttagaatcggggctcgtaatcaatatgattgtaatgcttttgtgctgaccctcagacaggtcgtggatacaatggctggggagtagtcgacgttcagagagtgcaaccaagtccatatgatcacgtacagagggtgattcgttactgcgacattgattccagggaaatattgaccctagaacttatggaatgaatatgacttaccctattttcagagagatgctgaggatgcttgttgctgctgggaaagatcaagaagccgcttacttacgtcagatgctcccctttcatatgaccgtttcgctaggattaaccaaattattaacgaggatcttcttagcgcattcagtctacctgatcagaatttcgatgttgttttgcataatctcattcaaggtaatttcggcgagacagatcccgtttactagaagtgtcctgggcctcaatttggtttgcctttgttcgaaggtttgagcctatcgctagatccgacctgcttgaggcagctcctttgattgaggctcgttatgccgcagagcttagtactatcagatggatgttcaacaacttagaatgatgagagctagagtgcctgatacagtcattaatgccacaccgagtcagtgttggaaagctgtcctcaaaaacgctccggaacctatcaagaacctgatgacctatctcactcattttcatttgttaatgtaagagatatcgtcagatggtcacaacagagggacattcaagaatcactcgcttacgttcttaatagggaagcttgggctatagctaatgattttgaggatttaatgctcgtcgatcacgtctacattcaaagacaatgttgccggaaccaagactggatgacatcaatgaattcagaagacaaggtttttttcatactaacatgattgatggcgcaccacctattggtgacgtgacccactacacttatgccattgctaacttcaagcaaatatgggacagtttagagcagcaataagaagaactttggatgacaatggatggattcagtttgggggaatgttaaggaatataaagattaagttctttgattcaagaccacccgacgaatccttacagcaatgccttacgtttacactgaagaagagcgtgacggagtgcgaatggttgcctttaaatacgcaacaaccgctactgcctattttttgctttataacgtggagtatagcaacacacagacaccttaataaccgttaacccaacatttaccatgacaaaaattcatatgagaaaaaagatcgtcagaagggtccgagctcccgacgtgttgtctcaggttaataagcgacttgtggcttataagggaaaatgcgacttatggatgttactaagtgccttaaaactggagtgcaattagcaaggccaacaatttaactcgagctgcag

SEQ ID NO: 36 - DNA, 1530 bp, synthetic construct
  misc_feature 1..1530: note = Plant Codon Optimised AHSV-1 VP5
  source 1..1530: mol_type = other DNA
cgtaccggtccatgggaaaatttacttcatttcttaagcgtgccggtacagcaacaaagagagctctgtctagtgacgctgctaaaaagatgtataaaacagctggtaaagcactgcaaagagttgtagaatcagaaacgggagcgctgctatagatggggtaatgcaaggaacaattcaaagcataattcaaggtgagaatttaggggattcaattaagcaggccgttattttgaatgtagcaggaacattggaaagtgctcctatcctctctcaccaggtgagcagatcttatacggaaaagtacacgaaattgaaaggatggaaaaagaagacagagttattgctgcccatgccaacgatatcgagaaggagtttggcaaagagcttatactataaggaaaattgttaaaggagaggaaaagattgaggagttggaggggaaggaagtcgaatacgtggagaaagcattagacggtttgctcaagataggtaagaaccaatcaaaaaggattactacttgtataaagctcttcagactgaggagcagcttcgtactgaggatgagatcgctatgatatccgactataaagagaagtttggagctctaaaggaggcaattcagattgagcaacaagcagctaataggaagctatacaggagatgctggacatttctgctgatgttattgaaacagcaagcgaagaggtgccaatatttggcgcagcagctgcaaatgttgtggcaactacccgagcaatcgaaggaggcctaaacttaaggagattgtcgataaactaactggtatagatctttcacatctgaaagttgcagaaatccacccacatgttatcggccaggctatcagtaaaaatgatgttgacgacaagaccctcgctgagcaattaagtccaaggtggacgctgtcgatgaaatgaaccaggaaaccgaacatgtgatacaaagtattctcccattggtcaaaagagaataccagaagcatgaaaacaagttcaacataaagatacatctgctctaaaaatacattctgaacatacaccgaaagttcacatttataccacaccctgggattccgataaggtgttcatctgtcgttgtatcgcaccacatcaccagcaacgatcattcatgatggatttgatttggaaatcgagtacgtctactatgaggatacaagtgttgaagcccatattatgcacggaggtgcagttatgattgaaggtaggggttttcgacaagcctactccgaattcatgtccgtgcttggtcaatgcctgaaacacctgaacttcataaaaggagaatgcagaggaatcaaggatctcaccccatttatatgggatctatggattatactatctctttcgatcaattatctaccaatgcctccaattggtgcacaatactgatcttcaaatgcattgcttacgtggtcctattaagtttcaaagaagaactattatgaatgccttgcttttcggcgtgaagattagctaactcgagctgcag

SEQ ID NO: 37 - DNA, 1065 bp, synthetic construct
  misc_feature 1..1065: note = Plant Codon Optimised AHSV-1 VP7
  source 1..1065: mol_type = other DNA
gctaccggtccatggatgcaatagcagctagggctttatccgttgttagggcttgtgtaaccgttactgacgcccgagtatctctcgacccaggcgttatggaaactttgggtatcgctattaaccgatataatgggctactaaccattcagtctcaatgaggccgcagacccaagctgaacgtaacgaaatgtttttcatgtgtacagatatggttttggctgccctgaatgtgcaaattggtaatattagtcctgactacgataagcattggctacagttggagcacttgccactaccgaaattccttacaatgtgcaggctatgaacgatatagtgagaattacaggtcagatgcagacttttgggccatccaaggttcaaactgggcctatgctggtgcagtcgaagtacaacaaagcggcagatactacgtgccgcaaggaagaacaagaggtggatacataaattctaacatagctgaagtctgtatggacgcaggagcagctggccaggtgatggactgttagctcctagaaggggagatgcagttatgatttatttcgtgtggcgaccactgcgtatcttttgcgatcctcagggtgcaagtttagagtctgctccaggtacatttgtcaccgttgatgagttaatgtagccgctggggatgtggttgcctggaacacaattgcacctgtcaatgttggaaatcccggcgctcgtaggtcaatccttcaattcgaggttctatggtatacttctctcgataggagctcgacaccgtcccagagttggctcccacacttactaggtgctatgcatatgtatcacctacatggcacgctctaagagccgtgattttccagcaaatgaacatgcagccaattaatccaccaatattccacccactgagcgtaatgagatcgttgcatatttgctcgtagctagtcttgccgatgtgtacgcagccttacgacctgattttagaatgaacggtgtagttgctcctgtgggacaaattaatagactttggttcttgcagcttatcattaactcgagctgcag

SEQ ID NO: 38 - DNA, 3147 bp, synthetic construct
  misc_feature 1..3147: note = Plant Codon Optimised AHSV-1 VP2
  source 1..3147: mol_type = other DNA
cgtaccggtccatggctagtgaattcggtattttgttaactgaacgtattttcgatgagacactagagaagactaactgcgacgtgatcattaccgaggagaagaaggtcaagagaaaagaggtagagggagttcttgtatgtttgggaagaaaccaatcatagattcggcttgtgtgaaggaaattacgacttggctttctctgatactatgtattgccaaactaaatgcgacggctcctaccccgtttttcctcactacatatgacgctttgagatacggcgttatgatagacaggaacgacaatcaggttagagttgatttagacgacaaaagactcatgaagataaagatccaaccgtacttaggagaaatgtatttttcaccagaaatactctactgtcttttgcaagaggcaggcactcgctttgggagtagatgatcttagacattcagttgatgttagaaatgaatttgaagagacaaatcaccagacaaaaggcgtgttgaacggaataacaaagggcattagaggtttggaaagagatggcataccaacgtatgcgaaaggaaggttctaggggaagatgcatcggtcatgatgatgatgtgatgtatcaattaattaagaaattgagatatgcatatgtaccctcaccattatgcacttaacgcaagatatgaggtgtcaaacccttctgctgctagaattaaagattggcttctcaaagttagggttaatgttggccgagcccaggaaaaggcagacagacggaccactcgctgaaatggctcgatcaattgagaatgatgaattaagcagacaggtggtcgatcaaattattcaatatggaggtcaatttagctcctgttctggtgctagagaggatgacaaccaataatattttactcgaatactgcgagagtttgacaacatttgttcacaggaagaagaggaaagaaggtgatgatttgactgccagaaatactttccgacaagccctgattaagagcatgcctcaatgaattcaaaaatcaaatgaaaatgactcgtggttgggggaactatacttttttttcctacatagataggttctcacgtatttacaatatgaatattgatcccaatggtaagttgtggattgacataaacaacagtctctgaacagctgaagaaaaagcaggaagagaacagagcccccttaactgtgcaaattgacggtgttcacatacgaaccgatgaaacatacggtacagttgatcattgggtggatgggtcgtgatacaattatgctgagagagacagagaagatgattaaagactatcgttttaaaaaattaaaacgtgaggagctgatttctgggatgaacaaactcgaagacggactccgttgcattgtttattgtttgatactcgctctatacgattactatgagggggatattgagggatttaagaagggtaccattgccagctcaatcgtggaaagtctcacaaatgtttccaaactttaggtcagatataattgataaatttggtatcagtttgaaggtcaaaactgaggctgaggaactgtttctgccaaagaatatgaagtcctcaatgaatgtcggaagaaagggggtataaatacaagttcggctggaaagacaatgaggagcgagttatgtctgattatggggagatcctgaccgagtctgtggagatcctatttaagaaactcctcaaaggtgagaaatgaaatcattgtcgatgatccgcagacctattttgaagatgatctcttcgttgacagggcaaacaagatcttcagcagagggggccaaactgtaaatcagttgattagcattaaggttaatgcacagtcaaaacgtcgagggaacaacttattttagtaagcgttttgtttcctattggttcaggattgagcattttagtattactactgccaaaaaaagaactgatattagggataagaaaactgaatataacaattgactttgaagatttcaagcctgcttgtattggagaattgggtatccatgcaagtacttacatttatcaagatcttttggtaggaaagtctaggggcgaaagagtaaaggacgcaaaggagttgtgtgatggatctatctttagcaaatttcgggtgttccaggtgctatgacagatgttggccagctagttgtgttgaagcagaaatctcacttaggtattatcttgttaccagtatattcgctcgttcttgaacgtgaagggctgtcattttctaagatacttatatcacttaaagatttttctgacaggttgtggtttcctacttacaagcacttttatgtagctgttgtccagaaagtactcagagatgatgaaggctgactatgtgctgttttgttcaagggtaagcgccattacaacaagacgagcaaccctgatggaattcagtactttcaaacaaatggttgggagcaccagactgctagatacacttttcctaactttctctttggataattttcgaacaagaaaatattgatgttgattttgctaataaatggcatcctctacttatttccactgagaaaggattgcgtgtcatcgccgtggatgtgttcaactccttcttacactagtacatctggatggcttccttatttggagcgtatttgtagtgaatcagctatggatagagctttaaccgcagacgaaatcaatcttaaacgatggtttgtagattactatatggaatgaaacttgaaggagagcagagccaagaatgtcttttaaatctgaagctcttatcacatggataggatcaaactgtggaggtgtaactgattacgttgtgcaacttctacctgtgagaaaaccaaaccaggtcttctgtggtagtttattccgaggacggaggcgaaaagtgggccgagtgggctttaagggactacttagaaatagatggaagcttgggtcttgtttttatcacacgtaaagctgtaaagataagagtaaactggagtgcgagatttgaagatctataatcgtggtagggttgataggctcatccttatttcttcaggtgtttacaccttcggtaataaattcttgttcagcaaattgctttcaaagtagaataactcgagctgcag

SEQ ID NO: 39 - DNA, 3176 bp, synthetic construct
  misc_feature 1..3176: note = Plant Codon Optimised AHSV-7 VP2
  source 1..3176: mol_type = other DNA
cgtaccggtccatggcatcagagtttggtatcttgtacacagatcagatttatgaacaaacacttgagaagactagttgcgatgttatagtcactaaggagaatgccgttaagagggtcgaaattgaggagtgctaggttatgagtggggagctacgaatcacaggcttgggctctgtgaaatcgaacatgttaaaaccatctccgaatttatgtatgaacagataaaatgtgaaggtgcctacccagtgttccctcattatgttatagacgcacttaagtacaataaggtgatagaaagaaatgacaaccaggtccggtagatcgtgatgatgaacgattgcaaaagatcaaaattcagccttactttggggaggcctttttctctccagaaacgtattcttcaacattttgtaaaagacaggctatccgtggtcacgttgctaaatgcgtactttcgctaaagatcggatagattttgaggagagtagcgctcaaactaaatacgtcaatgggaataaagttaagattttagaagagtggaaagggaggactgacgcacgtatgttaatgggggtcaacaagagaagtgcgttgcccatgaggtcgacccaatctaccagctcatcaaaaagatgcgttatggaatgatgtatccaacccattacatgttgtctcaacgatatcgaatcataagcgaagtaggaagatgggaattgaaaaatggcttcttgaaaaggtcgagaggggcgttcaacggggtgtcgccggggctaaaggtaatgagtcccttgtaaggctagaaaagctcatgcagaaagaagagctgagaattcagttatagagaatgtaattcgttatggttcacagttttcaacacacgccggagaaaagactaatgatatgcctctgtccgtgctgatcaagtactgtgagtccttgacaactttcgtgctaaaaagaaccgagaaggaggtgataaccagactgcaagggatgaaatccgaaaagctatggttcacaatatgcctacaatgaatcacaataatcctatgaaagtcacgaagaactttaagaacttttcttctttgcatatttggatggatttaaaagaaacaatggtgttgatatcaatcccaacaactcaacttggatcgagcacaagaagaagatggctgagaaacttaaggatgagcagcagaaaaaccaaacaggccgatgctcgtgccgattgatggagtttatgtgtctacctcagtggagtacggaacagtaacccactgggttgattgggtggtggatatcattatgacaactcaggttgagaggatgattaggaatatgacttcaagagacttgagcgaaagcaattgataagcggtatgaataagctggaggacggagtcaaatgttatgcttattgcctcatattagcattatatgattattatggtgatgctattaaggatttgcacaaggaactagggctggctctatagtcgagacaatatctcaaatgttccccgaatttcggtctgacgttgctgaaaagtttgggattaagttaacaataaaggatgagagcgaagattgtttgtgcagaaagacatgaatagtgaattcttggacgagggagaaatgggttacaagttcgttttcggatggaagaatacagatttcaaggttcaaagcaattatggagagatcgttagtgaggagtagagcgtctatttaaggttatacttgaggggaaggaatggtcaaacgaagtcgaagatcctgaagattattttgtggataacttgttcaataaaacaccggacgctgtatttgaacgagatggatggatggatctaatcggataatcgtaaagaataagaccaccttgcgggagggccaaaggcatttttctgcccggtttgttagctattggtacacctttgaaaaggttgaggtgggcagagctatggcagaattgatattcatgacagggagacaaagttctctgaattcgacgtcgatgattacaaaccctgctctgttgctgagatgggtcttcacagttctacctacatttaccaagacctgttgattggtcgaacagaggagagcatgttattgacgcaaaagaactggtttggtatgatattgctctgaccaactatggcacaactaggtactttgatcaatgttggcctagctcatgttccactacggagttgagttgagatatttccttatcacagaaatctttcagagatataggacggatgatagaagtagtttcgcagatattttagaaagaatgagaaaaaatgggtaccctagaaggaactttctaacctacaagcatactacgtggctgttattcaagaggttttcggggaccaacgtccaatcgacgtgtattcattctgcacggatattttcaagaaagagaggagaaggggcgttctttccatgtttcccacttttagtatttgattaaatccgaaaaacttattgatgcacttttcttgaatttctttttatgggtagtttttgaaatggagaatgtagacgtttctttcgctaataaacgacatcctcttctaatttctcatgatagggtctaagactcattggcgtggatctctttaattctgctctttccattagcatgggtggatggattccatacgtagagaggatttgccatgcagatcatgctgctagaaaattgaatgctgatgattgaaaattaagaggtggttcattgattactatatggatttgtcacttgatagacgtgccgaaccaagaatgtcctttaaatacgaaggtttagcaacatgggtgggtagtaactgtggcggtgttaggactatattatccaggaactaccaatgagaaaaccaaagccaggcttgctcattttggtatatggggaggatggtgatcctaaatgggttgaatgggctattaaggattttacccaaattgaaggtgcctcggatttatatacattgatcctattagtgtagtgaacaaatctactttcaggactagagaaatgaagatttacaatagaggcagactggacaggcttatattaattagctcaggtaactatacttcggaaataagtttctgctttctaagctgttatcaaaggcagaatgaggatccctcgagcag

SEQ ID NO: 40 - DNA, 1533 bp, synthetic construct
  misc_feature 1..1533: note = Plant Codon Optimised AHSV-7 VP5
  source 1..1533: mol_type = other DNA
cgtaccggtatgggaaagttcacatccttcctaaagcgtgcaggttctgctactaaaaaggcattgacatccgatgcagctaaaagaatgtacaagatggcaggacgaacactacaaaaggctgtagagtcagaagtcggaagtgcagcaattgatggagtaatgcaaggaacaatccaatctatcatccaaggagagaacctaggtgactccataaagcaagctgttatcctaaacgtcgcaggtacattagagtcagcacctgatccacttagtcctggtgaacaacttttgtataacaaggtatccgaactggaaagaatggagaaggaagatagagtcatcgagactcattccgagaaaatcgtcgaaaagtacggacgagaattagtcgatatcagaaagatcatgcggggagaagcacaagcagaaaagttagagggaaaggaaatgttctacatcgagaaggctctgaagggtattttacagattggcaaggaccaaagcgatcgaataacgagattataccgggctttacaaaccgaagaagagttgagaaccgcagatgaaacgaagataattagcgagtacagggaaaagttcaatgctctgaagcaggctattgagctggaacaacaggctacacatgaagaagctgtgcaagaaatgttggatctgagcgcagaagttcttgaaacagcagctgaagaagttccaattttcggtgcaggtgctgctaatgtagttgcaacaactagagctgttcagggtggtcttcgtttgaaagaaattatagacaagttgaccggcatagatctcagtcatctcaaggttgcagatattcacccacatattatagagaaagccatgctcaaagacgctgtgacggattctgatctcgctatggctgttaaaagcaaagtggatgtggtagatgaagtgaatgctgaaaccgaacatattattgacaccattatgccgctcgtgaagaaagagtacgacaaacacgagaataagtttcacattgagattcccagcgccctcaaaattcactcagagcacaccccaaaagtgcacatatatactactccgtgggattctgacaaagtgtttatctgcaggtgtattgcccctcaccatcagcagagatcttttatgatagggtttgacttggagattgagtttgttttctatgaggatacttctgttgagtcacatgttatgcatgggggggccatttcaattgaggggaggggttttaggcaggcttattctgagtttatgtcagccgcttggtcaatgccagctgcccctgaacttcataaacgtaggcttcagaggagtttggggtctcatcctatatatatgggcagtatggattatactgttagttatgagcagcttgtttctaatgccatgaaacttgtttatgatactgatttgcagatgcattgtttgcggggccccttgaaatttcagaggcgtactcttatgaatgcccttctttttggcgttcgtattgcctagctcgag

SEQ ID NO: 41 - DNA, 30 bp, synthetic construct
  misc_feature 1..30: note = Oligonucleotide
  source 1..30: mol_type = other DNA
gcaccggtatggaagaactcgctatcccaa

SEQ ID NO: 42 - DNA, 32 bp, synthetic construct
  misc_feature 1..32: note = Oligonucleotide
  source 1..32: mol_type = other DNA
gcctcgagtcaaacgttgaggagcttagtaag

SEQ ID NO: 43 - DNA, 31 bp, synthetic construct
  misc_feature 1..31: note = Oligonucleotide
  source 1..31: mol_type = other DNA
gcaccggtatggctgctcaaaatgagcaaag

SEQ ID NO: 44 - DNA, 30 bp, synthetic construct
  misc_feature 1..30: note = Oligonucleotide
  source 1..30: mol_type = other DNA
gcctcgagttaaacagttggagcagcaagc

SEQ ID NO: 45 - DNA, 34 bp, synthetic construct
  misc_feature 1..34: note = Oligonucleotide
  source 1..34: mol_type = other DNA
gcaccggtatgggaaagattattaagtccctctc

SEQ ID NO: 46 - DNA, 29 bp, synthetic construct
  misc_feature 1..29: note = Oligonucleotide
  source 1..29: mol_type = other DNA
gcctcgagtcaagcgttcctaaggaagag

SEQ ID NO: 47 - DNA, 30 bp, synthetic construct
  misc_feature 1..30: note = Oligonucleotide
  source 1..30: mol_type = other DNA
gcaccggtatggatacaattgctgctaggg

SEQ ID NO: 48 - DNA, 28 bp, synthetic construct
  misc_feature 1..28: note = Oligonucleotide
  source 1..28: mol_type = other DNA
gcctcgagtcacacataagcagccctag

SEQ ID NO: 49 - DNA, 30 bp, synthetic construct
  misc_feature 1..30: note = Oligonucleotide
  source 1..30: mol_type = other DNA
acttgttacgattctgctgactttcggcgg

SEQ ID NO: 50 - DNA, 28 bp, synthetic construct
  misc_feature 1..28: note = Oligonucleotide
  source 1..28: mol_type = other DNA
cgacctgctaaacaggagctcacaaaga

SEQ ID NO: 51 - DNA, 33 bp, synthetic construct
  misc_feature 1..33: note = Oligonucleotide
  source 1..33: mol_type = other DNA
caaattcgcgaccggtccatggctagtgaattc

SEQ ID NO: 52 - DNA, 35 bp, synthetic construct
  misc_feature 1..35: note = Oligonucleotide
  source 1..35: mol_type = other DNA
agttaaaggcctcgagttattctatctttgaaagc

SEQ ID NO: 53 - DNA, 29 bp, synthetic construct
  misc_feature 1..29: note = Oligonucleotide
  source 1..29: mol_type = other DNA
cgtaccggtccatggctagtgaattcggt

SEQ ID NO: 54 - DNA, 29 bp, synthetic construct
  misc_feature 1..29: note = Oligonucleotide
  source 1..29: mol_type = other DNA
gcagctcgagttattctatctttgaaagc

SEQ ID NO: 55 - DNA, 27 bp, synthetic construct
  misc_feature 1..27: note = Oligonucleotide
  source 1..27: mol_type = other DNA
ggtaccggtatgcaaggtaacgaacgt

SEQ ID NO: 56 - DNA, 27 bp, synthetic construct
  misc_feature 1..27: note = Oligonucleotide
  source 1..27: mol_type = other DNA
cagctcgagttaaattgttggccttgc

SEQ ID NO: 57 - DNA, 28 bp, synthetic construct
  misc_feature 1..28: note = Oligonucleotide
  source 1..28: mol_type = other DNA
cgtaccggtccatgggaaaatttacttc

SEQ ID NO: 58 - DNA, 27 bp, synthetic construct
  misc_feature 1..27: note = Oligonucleotide
  source 1..27: mol_type = other DNA
cagctcgagttagctaatcttcacgcc

SEQ ID NO: 59 - DNA, 28 bp, synthetic construct
  misc_feature 1..28: note = Oligonucleotide
  source 1..28: mol_type = other DNA
gctaccggtccatggatgcaatagcagc

SEQ ID NO: 60 - DNA, 27 bp, synthetic construct
  misc_feature 1..27: note = Oligonucleotide
  source 1..27: mol_type = other DNA
cagctcgagttaatgataagctgcaag

SEQ ID NO: 61 - DNA, 23 bp, synthetic construct
  misc_feature 1..23: note = Oligonucleotide
  source 1..23: mol_type = other DNA
ccatggcatcagagtttggtatc

SEQ ID NO: 62 - DNA, 24 bp, synthetic construct
  misc_feature 1..24: note = Oligonucleotide
  source 1..24: mol_type = other DNA
cctcattctgcctttgataacagc

SEQ ID NO: 63 - DNA, 18 bp, synthetic construct
  misc_feature 1..18: note = Oligonucleotide
  source 1..18: mol_type = other DNA
accggtatgggaaagttc

SEQ ID NO: 64 - DNA, 18 bp, synthetic construct
  misc_feature 1..18: note = Oligonucleotide
  source 1..18: mol_type = other DNA
ctcgagggcaatacgaac

SEQ ID NO: 65 - DNA, 1518 bp, synthetic construct
  misc_feature 1..1518: note = Plant codon optimised AHSV-3 VP5
  source 1..1518: mol_type = other DNA
atgggaaagttcacgagtttcctgaagagagcaggtagtgcaacaaagaaagcactaacttccgatgcagctaaaagaatgtacaagatggctggtaagacactacaaaaggtagtcgagagtgaggtaggaagtgcagcaattgatggagtaatgcaaggaacaatccaatcaatcatccaaggtgagaacttgggagattcaatcaagcaagcagtaatcctgaacgtagctggaacactagaatctgctccagatccattaagtccaggtgaacaactactgtataacaaggtttccgagatcgaaagagcagaaaaggaggatagagttatcgagacacacaacaagaagatcgttgagaagtacggagaggatctgttgaagatccgaaagatcatgaagggagaagctcaagcagaacaattagagggaaaggaaatggaatacgttgagaaggcattaggaggtttgttgaagataggtaaggaccaatccgagagaattacgcgattgtacagagctttacagacagaggaagatttaaggacgtccgatgaaacaagaattataagcgagtaccgtgagaagttcgacgcattgaagcaagcaattgagctggagcaacaggctacacatgaagaagctgttcaggaaatgttggatcttagtgctgaagttattgagactgctgctgaagaagttccagttttcggtgcaggtgctgctaatgttgttgctactactcgtgctattcagggtggtttaaaattgaaagagataatagacaagctcactggtatagacttgagccacttgaaggttgctgatattcaccctcacataattgagaaggctatgctcaaggacaaaattcccgacaatgagctcgcaatggctataaaaagcaaagtcgaggttgttgacgagatgaataccgagactgagcatgttatagaaagcataatgcctctcgtgaaaaaagaatacgaaaaacacgacaataaataccacgtgaatattccaagcgccctcaaaattcactcagaacatacccctaaagtccatatttataccaccccgtgggattctgataaagtgttcatttgccggtgtattgcccctcatcatcagcagaggtctttcatgattgggtttgatctcgaaattgaatttgtgttttatgaagatacctctgtcgtgggccatattatgcatgggggcgccgtgtctattgaagggcggggctttcgtcaggcttattctgaatttatgaatgccgcctggtctatgccttcaactcctgaacttcataaaaggaggcttcagcgttctcttggctcacatccgatttatatggggtcaatggattatactatttcttatgaacagcttgtgtcaaatgccatgaaacttgtctatgataccgatcttcagatgcattgtttgaggggcccccttaaatttcagaggcgaactcttatgaatgcccttctttttggggtgaaagtcgcctga

SEQ ID NO: 66 - protein, 505 aa, African horse sickness virus
MGKFTSFLKRAGSATKKALTSDAAKRMYKMAGKTLQKVVESEVGSAAIDGVMQGTIQSIIQGENLGDSIKQAVILNVAGTLESAPDPLSPGEQLLYNKVSEIERAEKEDRVIETHNKKIVEKYGEDLLKIRKIMKGEAQAEQLEGKEMEYVEKALGGLLKIGKDQSERITRLYRALQTEEDLRTSDETRIISEYREKFDALKQAIELEQQATHEEAVQEMLDLSAEVIETAAEEVPVFGAGAANVVATTRAIQGGLKLKEIIDKLTGIDLSHLKVADIHPHIIEKAMLKDKIPDNELAMAIKSKVEVVDEMNTETEHVIESIMPLVKKEYEKHDNKYHVNIPSALKIHSEHTPKVHIYTTPWDSDKVFICRCIAPHHQQRSFMIGFDLEIEFVFYEDTSVVGHIMHGGAVSIEGRGFRQAYSEFMNAAWSMPSTPELHKRRLQRSLGSHPIYMGSMDYTISYEQLVSNAMKLVYDTDLQMHCLRGPLKFQRRTLMNALLFGVKVA

SEQ ID NO: 67 - DNA, 3156 bp, synthetic construct
  misc_feature 1..3156: note = Plant codon optimised AHSV-6 VP2
  source 1..3156: mol_type = other DNA
atggcttctgaattcggtatcttgatctgcgataagctaaaggaaaacacactggagaagacaaactgcgatgttatcatcacaggagtaggaaaggttagtgtaagagaagaagatggtatcctaggttatgagtgggaagaaactaaccatagattgggattgtgtgaaatcgagaacacagtatccatcagtgatttcgtatacaagcagatcagatgcgaaggagcatatccaattctaccacattacgtaacagatgtcatcaagtacggaatggtaatcgatagaaacgaccaccaaatcagagtcgatagagatgagaagagtatcggaaagatccaaatccaaccatacttcggagatatgtacttctccccagaatattaccctgcaactttcgttaagagagaacctctaccaatttccgtagatatgatcagagattacatcggagcaagaatgagaaagatcgaggcaagagcaggaagaattaaggaaggtggaggtaatttgttggaatgtgcaagaagatgggaaaaggcagcatatgaaagaatcgaaaacgaaagagcactacgatgtgtagtacacgaaacagatcctacataccaaattctgaagaagctacgattcggattcatataccctcactactacgtcttgaacacggactataacccaactactgtaacgagaacttctcgaattaacgattggctgttgaaggagaaaacgcaaggagttgttaaggcagctgaagcatattcagataacgcagaattgaaaaccctagcagaacgaatggaagaggaggaactaactgttgatattattcgggctgttattaggtacggagcaaagtacgctactcgatctggtatgcgagaagatacattatcactacaagaactggatagatactgcgattctctaacaacgttcgttcacaagaaaaagaaagatgaaggagatgatgaaacggctagaacaattattcgaaaccagtggattaagggaatgccacgaatggatttcaagaaggaaatgaagataacgaggggtcctattgctaactggtcattcttcatgtctatagacgctttcaagcgtaacaacaaggttgatattaaccctaatcatcagacctggaaggatcacatcaaagaggttaccgatcaaatgaatcgggcacaacaaggtaataataacaagcctctgaagattcagattgacggagttagcatactcaccaatgagaagtacggtaccgttggtcattgggttgattgggttgttgacttaattatgctggctcaagttaagatgttgataaaggagtacaagtttaagcgtctgaatagccagaatctgatgtccggtatgaacaagctggtaggtgcattaagatgttacgcttactgtttgattctcgctttgtacgattactacggtcaagatattgagggtttcaagaaaggatccaattcctccgctatactggaaactgttattcagatgttcccaaatttcaaacaggagattcaggctaatttcgggataaacctgaacataaaagacaagaaacagtccctctttgtggagcggacaatgcactccgattttagcagcaatgaagaatatggctacaaatttgtgtttgggtgggctgctagaggagaagaagttttgagtaattatggggatattctcagcgatgaagttgaggaactcttcaccaagcttagaaagaaagagcactgggataaagttgttgaggatccagaatcttattttgtcgacgagctgtatcagaagaatcctgctgaagtgttctttagcgctggttatgatactgaccaaaatgttgttattgacggaaaaatgaccgagggagttacctatttttctaaaaggtttgtgagctattggtatcgtgtggagaaaataaccaccaaacatctcgaatttctcaatgaggagggtcgtaaagtggctcagtttgactttgaggactacaaacctatggctattggagaaatgggtattcatgctagcacctataagtatgagagcctccttcttggtaaaaatcgtggtcagaaagttaaagactctattgccctctgcaattatgacctcgctcttactaattttgaggtctctaggcgtcaagattgctgttggatttcatcttgctcagctattgaactctctatgcgtgctaatattactattgccatttttaggaggattgaggacaggcggtatgaaagttttgccaaaatactctcaggtctttcacagcagcaagacctttactttcctacttataagcattattatttgtttgtgcttcagaaagtgcttcgggacgagaggaggatagaccagaataggatgtgtacagagttgtttgacatacaaaggcgtaggggtattttgcttagttttactactttgaggttttggaatgactcagagtttttgggcgacactttgatgatgaattttcttttgtgggtggtctttgagatggagaatattgatgtggattatgggaagaaatggcatccgttgttggtgagttcagagaaagggcttcgagtgatagccgttgatgtgttcaattcaatgatgggggtttcaactagtggttggcttccgtatgttgaacggatttgttctgaatctgatatgcggagacgtcttaatgccgatgaattagagttgaaaaggtggttctttgattattatgccacattgttgcttgagaggaggggtgaaccgcgtttatctttcaaatatgagggcttaacaacttggattggctcaaattgtggcggcgttcgtgattatgtggtccaattacttcccatgcgtaaacccaaacccgggcttctttgtatagcctatggcgatgatgtcaatgtccaatgggtggaacatgagttaagggattttcttacacatgagggcagtcttgggttagtggtcatttctggcaaaatgttagtgaataagagtaaacttagggtcaggaatcttaaaatatataatcgggggactcttgattctctttttcttataagtgggggctcttatacatttgggaataagtttttactttcaaaattaatggccaaagccgagtga

SEQ ID NO: 68 - protein, 600 aa, African horse sickness virus
MASEFGILICDKLKENTLEKTNCDVIITGVGKVSVREEDGILGYEWEETNHRLGLCEIENTVSISDFVYKQIRCEGAYPILPHYVTDVIKYGMVIDRNDHQIRVDRDEKSIGKIQIQPYFGDMYFSPEYYPATFVKREPLPISVDMIRDYIGARMRKIEARAGRIKEGGGNLLECARRWEKAAYERIENERALRCVVHETDPTYQILKKLRFGFIYPHYYVLNTDYNPTTVTRTSRINDWLLKEKTQGVVKAAEAYSDNAELKTLAERMEEEELTVDIIRAVIRYGAKYATRSGMREDTLSLQELDRYCDSLTTFVHKKKKDEGDDETARTIIRNQWIKGMPRMDFKKEMKITRGPIANWSFFMSIDAFKRNNKVDINPNHQTWKDHIKEVTDQMNRAQQGNNNKPLKIQIDGVSILTNEKYGTVGHWVDWVVDLIMLAQVKMLIKEYKFKRLNSQNLMSGMNKLVGALRCYAYCLILALYDYYGQDIEGFKKGSNSSAILETVIQMFPNFKQEIQANFGINLNIKDKKQSLFVERTMHSDFSSNEEYGYKFVFGWAARGEEVLSNYGDILSDEVEELFTKLRKKEHWDKVVEDPESYFV

SEQ ID NO: 69 - DNA, 36 bp, synthetic construct
  misc_feature 1..36: note = Oligonucleotide
  source 1..36: mol_type = other DNA
caaattcgcgaccggtccatggttcagaattcggtg

SEQ ID NO: 70 - DNA, 35 bp, synthetic construct
  misc_feature 1..35: note = Oligonucleotide
  source 1..35: mol_type = other DNA
agttaaaggcctcgagtcatttctcggttttggcc

SEQ ID NO: 71 - DNA, 36 bp, synthetic construct
  misc_feature 1..36: note = Oligonucleotide
  source 1..36: mol_type = other DNA
caaattcgcgaccggtccatggcttctgaattcggt

SEQ ID NO: 72 - DNA, 34 bp, synthetic construct
  misc_feature 1..34: note = Oligonucleotide
  source 1..34: mol_type = other DNA
agttaaaggcctcgagtcactcggctttggccat

SEQ ID NO: 73 - DNA, 20 bp, synthetic construct
  misc_feature 1..20: note = Oligonucleotide
  source 1..20: mol_type = other DNA
ggttttcgaacttggagaaa

SEQ ID NO: 74 - DNA, 24 bp, synthetic construct
  misc_feature 1..24: note = Oligonucleotide
  source 1..24: mol_type = other DNA
agaaaaccgctcaccaaacataga

SEQ ID NO: 75 - DNA, 20 bp, synthetic construct
  misc_feature 1..20: note = Oligonucleotide
  source 1..20: mol_type = other DNA
ggttttcgaacttggagaaa

SEQ ID NO: 76 - DNA, 24 bp, synthetic construct
  misc_feature 1..24: note = Oligonucleotide
  source 1..24: mol_type = other DNA
agaaaaccgctcaccaaacataga

SEQ ID NO: 77 - DNA, 1050 bp, synthetic construct
  misc_feature 1..1050: note = Oligonucleotide
  source 1..1050: mol_type = other DNA
atggacgctatcgctgctagggcactctctgttgtgagggcttgtgttactgtgactgatgctagggtgtctcttgatcctggtgttatggaaactttgggtattgctatcaatagatataacggacttacaaaccattctgtttcaatgagacctcaaacccaggcagaaaggaatgagatgtttttcatgtgtaccgatatggttcttgctgcattgaatgtgcaaattggtaacatctcacctgattatgatcaggctcttgcaacagttggagctttggcaactacagaaattccatacaatgttcaagctatgaacgatattgtgagaatcactggtcaaatgcagacatttggaccttctaaggttcaaactggtccatatgctggagctgtggaagttcaacagtcaggaaggtattacgtgccacagggaagaacaaggggtggatacattaatagtaacatcgctgaggtttgtatggatgcaggtgctgcaggacaagtgaatgctcttttggctcctagaagaggagatgctgttatgatctattttgtgtggagacctcttaggatcttctgcgatccacagggtgctagtttggagtctgcaccaggaacatttgttaccgtggatggtgttaatgtggctgcaggagatgttgtggcttggaacacaattgcacctgttaatgtgggtaacccaggagctagaaggagtattttacaattcgaagttctctggtacacctcattagatagaagtctcgatactgttcctgagcttgctccaaccttgactagatgctatgcatacgtttctcatacttggcacgctcttagggcagtgatttttcaacagatgaatatgcagccaattaaccctccaatcttccctccaacagaaaggaatgagattgttgcttatcttctcgtggcttctttagcagatgtttacgctgcacttagacctgatttcgcgatgaatggtgttaatccgatgccagggccgatcaatagggctttggttcttgctgcttatcattag

SEQ ID NO: 78 - protein, 349 aa, synthetic construct
  REGION 1..349: note = Peptide
MDAIAARALSVVRACVTVTDARVSLDPGVMETLGIAINRYNGLTNHSVSMRPQTQAERNEMFFMCTDMVLAALNVQIGNISPDYDQALATVGALATTEIPYNVQAMNDIVRITGQMQTFGPSKVQTGPYAGAVEVQQSGRYYVPQGRTRGGYINSNIAEVCMDAGAAGQVNALLAPRRGDAVMIYFVWRPLRIFCDPQGASLESAPGTFVTVDGVNVAAGDVVAWNTIAPVNVGNPGARRSILQFEVLWYTSLDRSLDTVPELAPTLTRCYAYVSHTWHALRAVIFQQMNMQPINPPIFPPTERNEIVAYLLVASLADVYAALRPDFAMNGVNPMPGPINRALVLAAYH

SEQ ID NO: 79 - DNA, 1050 bp, synthetic construct
  misc_feature 1..1050: note = Oligonucleotide
  source 1..1050: mol_type = other DNA
atggacgctatcgctgctagggcactctctgttgtgagggcttgtgttactgtgactgatgctagggtgtctcttgatcctggtgttatggaaactttgggtattgctatcaatagatataacggacttacaaaccattctgtttcaatgagacctcaaacccaggcagaaaggaatgagatgtttttcatgtgtaccgatatggttcttgctgcattgaatgtgcaaattggtaacatctcacctgattatgatcaggctcttgcaacagttggagctttggcaactacagaaattccatacaatgttcaagctatgaacgatattgtgagaatcactggtcaaatgcagacatttggaccttctaaggttcaaactggtccatatgctggagctgcagaagttcaacagtcaggaaggtattacgtgccacagggaagaacaaggggtggatacattaatagtaacatcgctgaggtttgtatggatgcaggtgctgcaggacaagtgaatgctcttttggctcctagaagaggagatgctgttatgatctattttgtgtggagacctcttaggatcttctgcgatccacagggtgctagtttggagtctgcaccaggaacatttgttaccgtggatggtgttaatgtggctgcaggagatgttgtggcttggaacacaattgcacctgttaatgtgggtaacccaggagctagaaggagtattttacaattcgaagttctctggtacacctcattagatagaagtctcgatactgttcctgagcttgctccaaccttgactagatgctatgcatacgtttctcatacttggcacgctcttagggcagtgatttttcaacagatgaatatgcagccaattaaccctccaatcttccctccaacagaaaggaatgagattgttgcttatcttctcgtggcttctttagcagatgtttacgctgcacttagacctgatttcgcgatgaatggtgttaatccgatgccagggccgatcaatagggctttggttcttgctgcttatcattag

SEQ ID NO: 80 - protein, 349 aa, synthetic construct
  REGION 1..349: note = Peptide
MDAIAARALSVVRACVTVTDARVSLDPGVMETLGIAINRYNGLTNHSVSMRPQTQAERNEMFFMCTDMVLAALNVQIGNISPDYDQALATVGALATTEIPYNVQAMNDIVRITGQMQTFGPSKVQTGPYAGAAEVQQSGRYYVPQGRTRGGYINSNIAEVCMDAGAAGQVNALLAPRRGDAVMIYFVWRPLRIFCDPQGASLESAPGTFVTVDGVNVAAGDVVAWNTIAPVNVGNPGARRSILQFEVLWYTSLDRSLDTVPELAPTLTRCYAYVSHTWHALRAVIFQQMNMQPINPPIFPPTERNEIVAYLLVASLADVYAALRPDFAMNGVNPMPGPINRALVLAAYH

SEQ ID NO: 81 - protein, 956 aa, Bluetongue virus
MEEFVIPVYSETDIPYSLLSHYPLAIRTDVKIADTDEGHDVVKIPESDMIDVPRVDIVEALAAKPMRNDGIVVPRLLDITLRAYDDRKAIKSARGVEFMTNAKWMKWAIDDRMDIQPLKVAIDHYNAVNHQLFNCIVKARSANADTIYYDYFPLESKVKKCNHANLDLLRSLTNTEMFHMLQGAAYSLKSNYELITNSERNSTEETYAPGVHNQIRLVRGTRIGYKGEAYSRFVSSLVQVRIQGRTPPEIVDDIARLNVIRTEWINAQFDSTKIRALELCKILSAIGRKMLNTHEEPKDEMDLSTKFQFKLDDKFKKTDSEHINIFNVRAPATHEGRFYALIAIAATDTQRGRIWRTNPYPCLRGALIAAECELGDVYHTLRQVYKWSLRQDYGRTEVPLENNKYVFSRINLFDSNLDVGDQVVHWMYEVDGPAETTYDNGYMCKTEREDEELVCKISEEKYKTMLDRMIQGGWDQERFKLYSILTDPNLLTIDFEKDAHLNIRSEFVLPSYFDQWIYSPMFNARLRITHGEIATRKSADPWNKRVVFGYIKASTESPEYALGQYFDMRIQFYGDALSSKQNQSAVFQYLSQQEDFPTLTSYAKGDVVCPHSGGALYTFRRVALMLMANYEKLSPDLHEGMEDYTYTHPSIGGAYQKRILEMRDFSQLICFIIDYIFERHDQLRDAKEARRILYLIQNLDEPQRLEMLNVTFPNFFRHFLKLKDVQLISDLNVINFLPLLFLVQDNISYWHRQWAVPMILYDDVIRLIPVEVGAYANRFGIKSFFNFTRFHPGDAKKRQKADDTHKEFGSISFNYYASTKIAQGGVHTPVVTTKLDTLKIHLSSLCAGLADSIVYTLPVAHPKKCIVLIIVGDDKLEPHVRSEQVVSKYYFSRRHVSGIVSICVGQDNQLKVYSSGIVRHRVCEKFILKYKCKVVLVKMPGYVFGNDELMTKLLNV

SEQ ID NO: 82 - protein, 956 aa, Bluetongue virus
MEEFVIPVFSERDIPYSLLNHYPLAIRIDVKVDDEDGRHNLIKIPESDMIDVPKLSVIEALNYRPKRNDGVVVPRLLDITLHAYDKRKSTKSAKGVEFTTDAKWMKWAIDDKMDIQPLKVTLDNHYSVNHQLFNCIVKARSANADTIYYDYYPLENGAKKCNHTNLDLLRSLTTTEMFHILQGAAYALKTTHELVAHSERESTSETYQVGTQRWIQLRKGTKIGYRGQPYERFISSLVQVIIKGRVPDEIRDEIAELNRIKDEWKNAAYDRTKIRALELCKILSAIGRKMLDVQEEPKDEMALSTRFQFKLDEKFIRTDQEHVNIFEVGGPATDDGRFYALIAIAATDTQQGRVWRTNPYPCLRGALIAAECELGDVYFTLRQTYKWSLRSEYGQRERPLEDNKYVFSRLNLFDTNLAIGDEIIHWRYEIYRPKETTHDDGYICVSQKDDDELLCEIDGDRYKEMFDRMIQGGWDQERFKLHNILTEPNLLTIDFEKDAYLGSRSELVFPPYYDKWINSPMFNAKLKIARGEIATRKVDDPWNNRAVHGYIKTSAESLGYVLGPYYDLRLQLFGDALSLEQKQSAVFEYMAQQDDFPALTDYTKEKNGCPHSGGTFYTFRKVALIILSSYERLDPSLHEGREHETYMHPAINDVFRRYALEMKEFSQLICFVFDYIFEKHVQLRNAKEARRIIYLIQNTSGVHRLDILRETFPNFLRHVMNLRDVKRICDLNVINFFPLLFLIQDNISYWHRQWSIPMILFGEVVRLIPIEVGAYANRFGFKSFLNFIRFHPGDSKKKQDADDTHKEFGSICFEYYTTTKISQGEIDVPVITSKLDTLKLHIASLCAGLADSLVYTLPVAHPKKSIVLIIVGDDKLEPQVRSEQIVNKYHYSRRHISGVVSICVDQNGQLKVHSMGITRHRICDKSILKYKCKVVLVRMPGHVFGNDELMTKLLNV

SEQ ID NO: 83 - protein, 955 aa, Bluetongue virus
MEEFVIPVYSEDEIPYALLSRYPLAIQTNVKIEDVEGKHNVVKIPESDMIDIPKLTIVEAMNYKPARNDGIVVPRLLDITLRAYDDRKSTKSARGIEFMTNARWMKWAIDDRMDIQPLKVTLDHYCSVNHQLFNCVVKANAANADTIYYDYFPLEDYKKRCNHTNLDLLRSLTNMELFHALQGAAYSIKSSYELVAYSERGSLEETYVVGQPKWIHLTRGTRIGNSGLSYERFISSMVQVSVNGKIPDEIANEIAQLNRIRAEWITATYDRGRIRALELCNILSTIGRKMLNTHEEPKDEMDLSTRFQFKLDEKFNRADSEHVNIFGVRGPATDEGRFYALIAIAATDTQKGRVWRTNPYPCLRGALVAAECELGDVYSTLRRVYRWSLRPEYGQHERQLENNKYVFNRINLFDSNLAVGDQIIHWRYEVKASAETTYDSGYMCRHEAEEDELLCKINEDKYKEMLDRMIQGGWDQERFKLHNILTDPNLLTIDFEKDAYLNSRSELVLPDYFDKWISSPMFNARLRITKGEIGTSKKDDPWNNRAVRGYIKSPAESLDFVLGPYYDLRLLFFGETLSLKQEQSAVFQYLSQLDDFPALTQLTGDAVCPHSGGALYTFRKVALFLIGNYEKLSPDLHEGMEHQRYVHPSTGGTYQKRVLEMKDPCQLTCFVIDYIFEKREQLRDTKEARYIVYLIQSLTGTQRLSVLRSTFPNFFQRLLMLKEIKFVRDLNVINFLPLMFLVHDNISYWHRQWSIPMVLFDDTIKLIPVEVGAYANRFGFKSFMNFTRFHPGELKKKQIAEDIHKEFGVVAFEYYTNTKISQGNVHTPVMTTKMDVLRVHLSSLCAGLADSVVYTLPVAHPKKCIVLIIVGDDKLEPHTRSEQIVSRYNYSRKHICGIVSVTIGQNSQLRVHTSGIVKHRVCDKFILKHKCKVILVRMPGYVFGNDELMTKLLNV

SEQ ID NO: 84 - protein, 956 aa, Bluetongue virus
MEEFVIPVFSEREIPYALINQYPLAIQTDVRVVDVDDNHNLVKIPESDMIDVPKLDIVSALNYKPTRNDGIVVPRLLDITLKAYDDRKSVKNARGVDFMTDAKWMKWAIDDRMDIQPLKITLDEHYSVNHQLFNCIVKAKTANADTIYYDYFPLEDRAKKCNHTNLELLRSLTTIEAFHILQGAAYSLKSNYDLIANSERESLEESYPIGSEKWVHLTRRTKIGNSGLSYNRSISSMVQVVVRGKVPDIIRGEITQLNRIRTEWIGASYDRTRIRALELCNILSAIGRKMMDTHEEPKDEMDLSTRFQFKLDEKFNTSDFEHVNIFRTSGAATNEGRFYALIAIAATDTQKGRVWRTNPYPCLRGALIASECELGDVYYTLRHVYRWSLRPEYGQRERQLEDNKYVFGRVNLFDSDLAVGDQIIHWQYEITEPVKTTYDDGYICNPEEKDDELLCKIDDERYKEMMERLIEGGWDQERFKLHSILTEPNLLTIDFEKDAYLNSRSELVFPNYFDKWINSPMFNARLRITHGEIGSSKTIDPWNRRVVYGYVKTSIESLDYALGRYYDIRLQLFGDTLSQKQTQSAVFTYLSEQDDFPALTNYSKGEAVCPHAGGAVYTFRKVALSLIANYEKLSPEMHEGLEHQMYVHPSANTTYQKQVKDMKDFSQLICFIIDCIFEKRVQIRGVGEARRIIYLIQNSTGSQRQEVLKKTFPNFFMRIFKLREVKRICDLSVINFLPLLFLVQDNISYWHRQWSVPMILFDDAVRLIPVEVGAYANRFGLKSFYNFVRFHPGDSKKKQDADDMHKEYGVACFEYYMNTKISQGGVNVPVVTSKLDTLKIHLASLCLGLADSIVYTLPVAHPKKCIVLIVVGDDKLDPQVRSEQVLSKYYYSRRHICGIVAVSVGQEGQLQVYSSGIVRHRICEKSILKYKCKVVLVRMPGHVFGNDELMTKLLNV